(19) United States
(12) Patent Application Publication
(10) Pub. No.: US 2022/0313626 A1
(43) Pub. Date: Oct. 6, 2022

(54) SMALL MOLECULE INHIBITORS FOR TREATING CANCER IN A SUBJECT HAVING TUMORS WITH HIGH INTERSTITIAL PRESSURE

(71) Applicant: GUANGZHOU MAXINOVEL PHARMACEUTICALS CO., LTD., Guangzhou (CN)

(72) Inventors: Yugang WANG, Shanghai (CN); Feilan WANG, Shanghai (CN); Nong ZHANG, Shanghai (CN)

(21) Appl. No.: 17/621,576

(22) PCT Filed: Jun. 23, 2020
(86) PCT No.: PCT/CN2020/097648

§ 371 Date: Dec. 21, 2021

(30) Foreign Application Priority Data
Jun. 24, 2019 (CN) PCT/CN2019/092485
Jun. 17, 2020 (CN) 202010554193.0

(51) Int. Cl. A61K 31/137; A61K 31/198; A61K 31/357; A61K 31/277; A61K 31/397; A61K 31/401; A61K 31/454; A61K 31/4196; A61K 31/4025; A61K 31/423; A61K 31/4545; A61P 35/02

(52) U.S. Cl. CPC A61K 31/137; A61K 31/198; A61K 31/357; A61K 31/277; A61K 31/397; A61K 31/401; A61K 31/454; A61K 31/4196; A61K 31/4025; A61K 31/423; A61K 31/4545; A61P 35/02

(57) ABSTRACT

Provided herein are methods for treating a cancer in a subject having a tumor with interstitial fluid pressure (IFP) of at least 10 mmHg, comprising administering to the subject a therapeutically effective amount of a compound or a pharmaceutically acceptable salt or prodrug thereof, which is an inhibitor of the interaction between the PD-1 receptor and its ligand PD-L1 and which is not a protein, alone, or in combination with other agents, e.g., in combination with the use of anti-PD-1/PD-L1 antibodies, in combination with an inhibitor of the CTLA-4/B7 interaction, or in combination with an inhibitor binding to VEFG.

The present invention claims the priority of the PCT/CN2019/092485 filed on Jun. 24, 2019, and priority of the CN202010554193.0 filed on Jun. 17, 2020, the contents of which are incorporated herein by its entirety.

### FIELD OF THE INVENTION

The invention relates to methods of treating patients having tumors characterized by high interstitial pressure, e.g., which are resistant to treatment with monoclonal antibodies to PD-L1, using small molecule inhibitors targeting the interaction of PD-1 and PD-L1. The invention also relates to methods of improving therapeutic effects and response rates in treating cancer.

### BACKGROUND

PD-1 (Programmed death 1, CD279) is a major immunosuppressive molecule. It is a member of the CD28 superfamily and was originally cloned from the apoptotic mouse T cell hybridoma 2B4.11. PD-1 is mainly distributed in immune-related cells, such as T cells, B cells and NK cells, and plays an important role in immune response processes, e.g., autoimmune diseases, tumors, infections, organ transplantation or allergies.

Programmed death-ligand 1 (PD-L1), also known as B7-H1, belongs to the B7 family and is widely distributed in peripheral tissues and hematopoietic cells. PD-L1 is mainly expressed in hematopoietic cells such as CD4 T cells, CD8 T cells, B cells, monocytes, dendritic cells (DCs), macrophages, and some non-hematopoietic cells, such as endothelial cells, islet cells and mast cells. PD-L1 is highly expressed in various tumors, such as lung cancer, gastric cancer, melanoma and breast cancer. Programmed death-1 (PD-1) is the major receptor for PD-L1.

PD-1/PD-L1 exerts a negative immunomodulatory effect. When PD-1 on the surface of immune cells interacts with PD-L1on the surface of cancer cells, for example, tumor cells, the interaction causes a series of signaling responses leading to inhibition of T lymphocyte proliferation and secretion of related cytokines, apoptosis of tumor antigen-specific T cells, and/or incapable immunization, ultimately suppressing the immune response and promoting the escape of tumor cells. Monoclonal antibodies targeting PD-1 or PD-L1 can break the immune tolerance of tumors by specifically blocking the interaction of PD-1/PD-L1, restore the killing function of tumor-specific T cells on tumor cells, and achieve clearance of tumors. Up to now, there are four PD-1 antibody drugs and four PD-L1 antibody drugs in China and in the US. The approved PD-1 antibody drugs include Merck's Keytruda® (referred to as K drug), Bristol-Myers Squibb's Opdivo® (referred to as O drug), Junshi Bioscience's Toripalimab and Innovent's Sintilimab. The approved PD-L1 antibody drugs include Atezolizumab® by Roche, Durvalumab® by AstraZeneca, Avelumab® by Pfizer and Merck (Germany), and Cemiplimab® by Regeneron. In addition, a number of other companies are developing PD-1/PD-L1 targeted antibody drugs.

Small molecule inhibitors binding to PD-1/PD-L1 are also actively developed. WO2018006795, which is incorporated herein by reference in its entirety, discloses novel small molecule inhibitors binding to PD-1/PD-L1. The small molecule inhibitors disclosed therein exhibit an anti-tumor effect in a mouse tumor model and are currently undergoing preclinical studies.

Many cancer patients benefit from monoclonal antibodies to PD-1/PD-L1 However, studies have found that PD-1/PD-L1 antibodies are not effective in all cancer patients. Clinical trial data show the effective response rate of PD-1/PD-L1 antibody alone is about 20%.

The reasons for this poor response rate are not entirely clear. Delivery of drugs into tumor tissue is affected by several factors, including blood flow within the tumor (perfusion), permeability of the capillary walls and of the tumor tissue, pressure gradients (convection), and concentration gradients (diffusion). Tumor blood vessels are often very “leaky” compared to normal blood vessels, as the basement membrane may not be continuous and the endothelial cells may be disorganized, so that larger molecules can easily pass through. Tumors may, however, be poorly vascularized and may contain collagen matrices, calcium deposits, or other barriers.

Some tumors display greatly elevated hydraulic pressure within the tumor tissue, making them less susceptible to treatment with drugs. The hydraulic pressure between tumor tissues increases and forms a transcapillary transport barrier for therapeutic drugs, reducing hydraulic conductivity, convection and diffusion into the tissue, and hindering the transport of drugs and oxygen in the interstitial space, so that the concentrations of drugs and oxygen in cancer cells are reduced, leading to a decrease in response rate.

There is a need to improve the effective response rate in cancer immunotherapy, particularly in the case of patients who do not respond to monoclonal antibodies to PD-1/PD-L1 and patients having tumors displaying elevated interstitial pressure.

### SUMMARY OF TEIE INVENTION

We have discovered that small molecule inhibitors of the PD-1/PD-L1 interaction are more effective than monoclonal antibodies to PD-1 or PD-L1 in treating tumors displaying elevated interstitial pressure.

In one aspect, the invention provides methods for treating a cancer in a subject with a tumor having interstitial fluid pressure (IFP) of at least 10 mmHg, comprising administering to the subject a therapeutically effective amount of a compound or a pharmaceutically acceptable salt or prodrug thereof, wherein the compound is an inhibitor of the interaction between the PD-1 receptor and its ligand PD-L1 and wherein the compound is not a protein.

In another aspect, the invention provides methods for treating a cancer in a subject with a tumor having interstitial fluid pressure (IFP) of at least 10 mmHg, comprising administering to the subject a therapeutically effective amount of a compound or a pharmaceutically acceptable salt or prodrug thereof, wherein the compound has a molecular weight (MW) less than 1500 Daltons.

In another aspect, the invention provides methods for treating a cancer in a subject with a tumor having interstitial fluid pressure (IFP) of at least 10 mmHg, comprising administering to the subject a therapeutically effective amount of a compound or a pharmaceutically acceptable salt or prodrug thereof, wherein the compound has an IC₅₀ of less than 100 nM in a PD-1/PD-L1 binding assay.

In another aspect, the invention provides methods for treating a cancer in a subject with a tumor having interstitial fluid pressure (IFP) at least 10 mmHg, comprising administering to the subject a therapeutically effective amount of a compound or a pharmaceutically acceptable salt or prodrug thereof, and an antibody binding to PD-1 and/or PD-L1, wherein the compound is an inhibitor of the interaction between the PD-1 receptor and its ligand PD-L1, optionally wherein the compound has a molecular weight (MW) less than 1000 g/mol, optionally wherein the compound has an IC₅₀ of less than 100 nM in a PD-1/PD-L1 binding assay.

In another aspect, the invention provides methods for treating a cancer in a subject with a tumor having interstitial fluid pressure (IFP) at least 10 mmHg, comprising administering to the subject a therapeutically effective amount of a compound or a pharmaceutically acceptable salt or prodrug thereof, and an antibody binding to PD-1 and/or PD-L1, wherein the compound is an inhibitor of the interaction between the PD-1 receptor and its ligand PD-L1, optionally wherein the compound has a molecular weight (MW) less than 1500 g/mol, optionally wherein the compound has an IC₅₀ of less than 100 nM in a PD-1/PD-L1 binding assay.

In another aspect, the invention provides methods for treating a cancer in a subject with a tumor having interstitial fluid pressure (IFP) at least 10 mmHg, comprising administering to the subject a therapeutically effective amount of a compound or a pharmaceutically acceptable salt or prodrug thereof, wherein the compound is an inhibitor of the interaction between the PD-1 receptor and its ligand PD-L1, wherein the compound has a molecular weight (MW) less than 1500 Daltons, or wherein the compound has an IC₅₀ of less than 100 nM in a PD-1/PD-L1 binding assay and an inhibitor of the CTLA-4/B7 interaction, e.g., anti-CTLA4 monoclonal antibody and/or CTLA-4-Ig.

In another aspect, the invention further provides methods for treating a cancer in a subject with a tumor having interstitial fluid pressure (IFP) at least 10 mmHg, comprising administering to the subject a therapeutically effective amount of a compound or a pharmaceutically acceptable salt or prodrug thereof and an inhibitor binding to vascular endothelial growth factor (VEGF), wherein the compound is an inhibitor of the interaction between the PD-1 receptor and its ligand PD-L1, wherein the compound has a molecular weight (MW) less than 1500 Daltons, or wherein the compound has an IC₅₀ of less than 100 nM in a PD-1/PD-L1 binding assay.

In another aspect, the invention provides small molecule inhibitors of the PD-1/PD-L1 interaction for use in treating tumors displaying elevated interstitial fluid pressure, e.g., at least 10 mmHg.

### DETAILED DESCRIPTIONS OF THE INVENTION

The invention provides methods for treating a cancer in a subject with a tumor having interstitial fluid pressure (IFP) of at least 10 mmHg, comprising administering to the subject a therapeutically effective amount of a compound or a pharmaceutically acceptable salt or prodrug thereof The invention also provides methods for treating a cancer in a subject with a tumor having interstitial fluid pressure (IFP) of at least 10 mmHg, comprising administering to the subject a therapeutically effective amount of a compound or a pharmaceutically acceptable salt or prodrug thereof and an antibody binding to PD-1/PD-L1. The invention further provides methods for treating a cancer in a subject with a tumor having interstitial fluid pressure (IFP) of at least 10 mmHg, comprising administering to the subject a therapeutically effective amount of a compound or a pharmaceutically acceptable salt or prodrug thereof and an inhibitor of the CTLA-4/B7 interaction. The invention further provides methods for treating a cancer in a subject with a tumor having interstitial fluid pressure (IFP) of at least 10 mmHg, comprising administering to the subject a therapeutically effective amount of a compound or a pharmaceutically acceptable salt or prodrug thereof and an inhibitor binding to VEGF.

Unless specifically stated otherwise herein, references made in the singular may also include the plural. For example, “a” and “an” may refer to either one, or one or more.

Listed below are definitions of various terms used to describe the present disclosure. These definitions apply to the terms as they are used throughout the specification (unless they are otherwise limited in specific instances) either individually or as part of a larger group. The definitions set forth herein take precedence over definitions set forth in any patent, patent application, and/or patent application publication incorporated herein by reference.

As used herein, “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, an “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the individual or subject is a human.

As used herein, “protein” means a compound consisting of at least 50 amino acids linked in a chain, the alpha-carboxyl group of each amino acid being joined to the alpha-amino group of the next by an amide bond, including protein multimers, e.g., antibodies, post-translationally modified proteins, e.g., glycosylated proteins, and proteins complexed with metals.

As used herein, “therapeutically effective amount” is intended to include an amount of a compound of the present disclosure alone or an amount of the combination of compounds claimed or an amount of a compound of the present disclosure in combination with other active ingredients effective to act as an inhibitor of the interaction of PD-1 and PD-L1, or effective to treat or prevent cancer.

As used herein, “treatment” or “treating” is an approach for obtaining beneficial or desired results including and preferably clinical results. For purposes of this invention, beneficial or desired clinical results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals.

Reference to “about” a value or parameter herein includes (and describes) embodiments that are directed to that value or parameter per se. For example, description referring to “about X” includes description of “X.”

It is understood that embodiments, aspects and variations of the invention described herein include “consisting” and/or “consisting essentially of” embodiments, aspects and variations.

Cancer drug therapy has gone through several stages from chemotherapy, to targeted therapy, to immunotherapy during the past 50 years. While chemotherapy and targeted therapy involve in drugs to directly target cancer cells, immunotherapy relies on drugs to modulate the patient's own immune system which, in turns, kills the tumor cells. Thus, there are differences in therapeutic effects and toxicity profiles among the three therapies. Currently, immunotherapy is gaining the leadership role due to its durable response to some tumors and low occurrence of side effects.

The most successful immunotherapy is immuno checkpoint inhibition (ICI). Since the 2011 FDA approval of ipilimumab (anti-CTLA4) for the treatment of metastatic melanoma, more immuno checkpoint inhibitors, all targeting the PD-1/PD-L1 axis, have been approved for the treatment of a broad range of tumor types. ICI targets inhibitory ligand-receptor interactions between T cells and immunosuppressive cells within the tumor microenvironment (TME), in particular, interactions mediated by tumor cells (Pardon, D. M. Nat. Rev. Cancer 2012, 12, 252-264). Malignant tumors often co-opt immune suppressive and tolerance mechanisms to avoid immune destruction. Anti-PD1/L1 antibodies inhibit T cell-negative costimulation to unleash antitumor T-cell responses that recognize tumor antigens.

PD-1, expressed upon activation of T and B lymphocytes, regulates T-cell activation through interaction with PD-L1 and PD-L2. (Wei, S. C. et al Cancer Discov. 2018, 8(9), 1069-86). When binding with PD-L1, PD-1 primarily transmits a negative costimulatory signal through the tyrosine phosphatase SHP2 to attenuate T-cell activation. Therefore, inhibition of PD-1/PD-L1 axis with anti-PD-1/L1 antibodies stops the negative costimulatory signal and restores the T-cell activation to achieve tumor inhibition.

Extensive studies of the commercially available PD-1/L1 antibody drugs have revealed how these antibody drugs interact with their target protein. The binding structures of anti-PD-1 antibody Pembrolizumab with PD-1 protein have been disclosed, (Tan, S. et al Protein Cell 2016, 7:866-877). Crystal structures of Pembrolizumab fragment complexed with hPD-1 showed the molecular basis of therapeutic antibody-based immuno checkpoint inhibition of tumors. The interaction of Pembrolizumab with hPD-1 is mainly located on two regions: the flexible C′D loop and the C, C′ strands.

The protein binding model of anti-PD-L1 antibody drugs such as Durvalumab has also been published in Tan, S. et al Protein Cell 2017. The molecular basis of Durvalumab-based PD-1/PD-L1 blockade is that the unbiased binding of Durvalumab VH and VL to PD-L1 provides steric clash to abrogate the binding of PD-1/PD-L1. This is quite different from anti-PD-1 antibody Pembrolizumab with its residues participating in competitive binding to the ligand.

These pieces of binding information of anti-PD1/L1 antibody drugs at molecular level provide critical starting point for us to design the next generation immuno checkpoint inhibitors. Since the current antibody ICI therapy works for only 20-30% of the patients, it is in great need to develop the next generation drugs as soon as possible. Therefore, the next generation immuno checkpoint inhibitors require: 1) wider treatment response to more tumors than the current antibody therapy; 2) patient friendly oral dosing regimen; 3) effective brain penetration, and 4) shorter half life for side effect management.

In one aspect, the invention provides a method (Method 1) for treating a cancer in a subject having a tumor with interstitial fluid pressure (IFP) of at least 10 mmHg, comprising administering to the subject a therapeutically effective amount of a compound or a pharmaceutically acceptable salt or prodrug thereof, wherein the compound is an inhibitor of the interaction between the PD-1 receptor and its ligand PD-L1 and wherein the compound is not a protein:
1.1 Method 1, wherein the compound binds to PD-L1.
1.2 Method 1 or 1.1, wherein the compound has a molecular weight of less than 1500 Daltons.
1.3 Any foregoing method, wherein the compound has an IC₅₀ less than 100 nM in a PD-1/PD-L1 binding assay, e.g., an assay as described in WO2018006795.
1.4 Method 1, wherein the compound is an aromatic acetylene or aromatic ethylene PD-L1 inhibitor, e.g., as described in WO2018006795, incorporated herein by reference.
1.5 Method 1, wherein the compound is an aromatic ethylene or aromatic ethyl PD-L1 inhibitor, e.g., as described in PCT/CN2018/123066, incorporated herein by reference.
1.6 Method 1, wherein the compound is a benzyl phenyl ether PD-L1 inhibitor. e.g., as described in WO2015034820 and/or WO2015160641, the contents of which applications are incorporated herein by reference, for example, BMS-1001 or BMS-1166.
1.7 Method 1, wherein the compound is a derivative of aromatic ethylene or aromatic ethyl of formula (I), or a pharmaceutically acceptable salt, a metabolite, a metabolic precursor or a prodrug thereof;
wherein,

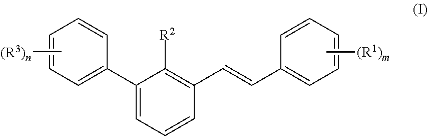

[symbol] is a single bond or a double bond;
each of R¹ is the same or different, is independently selected from deuterium, halogen, a substituted or unsubstituted hydroxyl, a substituted or unsubstituted amino, a substituted or unsubstituted alkyl, or a substituted or unsubstituted alkoxyl; or the two adjacent R¹(s) together with the carbon atoms on the phenyl to which they are attached to form a 5- to 7-membered carboncyclyl or heterocyclyl together; the heterocyclyl is a heterocyclyl wherein the heteroatom is selected from the group consisting of oxygen and/or nitrogen, the number of the heteroatom(s) is 1 to 4;
R² is selected from a substituted or unsubstituted alkyl or a halogen;
each of R³ is the same or different, is independently selected from deuterium, halogen, a substituted or unsubstituted alkylthio, a substituted or unsubstituted hydroxyl, a substituted or unsubstituted amino, a substituted or unsubstituted alkyl, a substituted or unsubstituted alkoxyl,

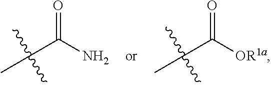

wherein R^{1a} is C₁-C₄ alkyl, or the two adjacent R³(s) together with the carbon atoms on the phenyl to which they are attached to form a 5- to 7-membered carbocyclyl or heterocyclyl together; the heterocyclyl is a heterocyclyl wherein the heteroatom is selected from the group consisting of oxygen and/or nitrogen, the number of the heteroatom(s) is 1 to 4; when two R³(s) are adjacent, and two R³(s) and two carbon atoms connected with them to form a 5- to 7-membered carboncyclyl or heterocyclyl together, the carboncyclyl or heterocyclyl is further substituted by one or more C₁-C₄ alkyl;
the substituted alkyl in each of R¹, R² and R³, the substituted alkoxyl in each of R¹, and R³ and the substituted alkylthio in each of R³, is selected one or more from the group consisting of halogen, C₁₋₄ alkyl, hydroxyl,

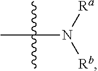

C₁₋₄ alkoxyl, C₁₋₄ carboxyl, C₁₋₄ ester group and C₁₋₄ amide group; when there are many substituents, the substituents are the same or different; R^{a} and R^{b} are independently selected from halogen, or, a substituted or unsubstituted alkyl; R^{a} and R^{b} can also be independently selected from hydrogen, or, a substituted or unsubstituted alkyl, in R^{a} or R^{b} , the substituents of a substituted alkyl are selected from the group consisting of halogen, C₁-C₄ alkyl, hydroxyl,

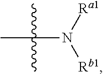

C₁-C₄ alkoxyl, C₁-C₄ carboxyl, C₁-C₄ ester group or C₁-C₄ amide group; R^{a1} and R^{b1} are independently selected from hydrogen or C₁-C₄ alkyl;
in each of R¹ or R³, the substituents of a substituted hydroxyl or a substituted amino are selected one or more from the group consisting of C₁₋₄ alkyl, C₁₋₄ alkoxyl, C₁₋₄ carboxyl, C₁₋₄ ester group and C₁₋₄ amide group;
m is 1, 2, or 3;
n is 0, 1, 2 or 3;
when [symbol] is a double bond, m is 2, and the two R¹(s) are occupied in ortho and meta positions of the phenyl, respectively, the two R¹(s) are the same or different;
when [symbol] is a double bond, m is 3, the two R¹(s) are adjacent, and the two adjacent R¹(s) together with the carbon atoms on the phenyl to which they are attached to form a 5- to 7-membered heterocyclyl together; or the derivative of aromatic ethylene or aromatic ethyl group of formula (I),

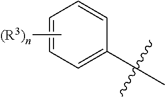

is replaced by a substituted or unsubstituted hetero aromatic ring, the heteroatom of the hetero aromatic ring is selected from oxygen, nitrogen or sulfur, the number of heteroatoms is 1-4; the substituents of a substituted hetero aromatic ring are selected one or more from the group consisting of halogen, C₁₋₄ alkyl, hydroxyl,

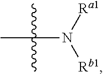

C₁₋₄ alkoxyl, C₁₋₄ carboxyl, C₁₋₄ ester group or C₁₋₄ amide group; the substituents of a substituted hetero aromatic ring are selected one or more from the group consisting of halogen, C₁₋₄ alkyl, hydroxyl,

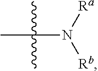

C₁₋₄ alkoxyl, C₁₋₄ carboxyl, C₁₋₄ ester group or C₁₋₄ amide group; when there are many substituents, the substituents are the same or different; R^{a1} and R^{b1} are independently selected from halogen, or, a substituted or unsubstituted alkyl; R^{a} and R^{b} are independently selected from hydrogen, or, a substituted or unsubstituted alkyl; in R^{a} or R^{b}, the substituents of a substituted alkyl are selected one or more from the group consisting of halogen, C₁-C₄ alkyl, hydroxyl,

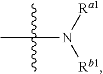

C₁-C₄ alkoxyl, C₁-C₄ carboxyl, C₁-C₄ ester group or C₁-C₄ amide group; R^{a1} and R^{b1} are independently selected from hydrogen or C₁-C₄ alkyl; and
the derivative of aromatic ethylene or aromatic ethyl group of formula (I) does not contain the following compounds:

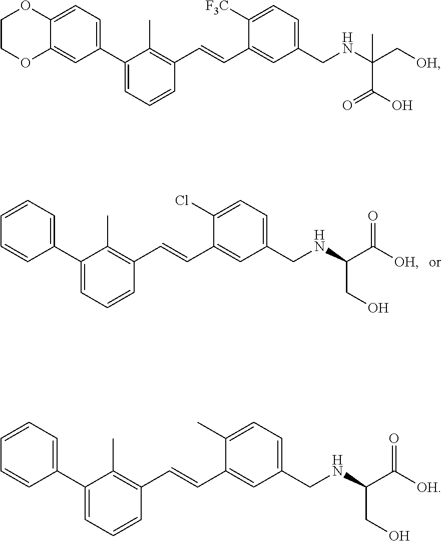 [SMILES: Cc1c(/C=C/c2cc(CNC(C)(CO)C(=O)O)ccc2C(F)(F)F)cccc1-c1ccc2c(c1)OCCO2 ; Cc1c(/C=C/c2cc(CN[C@H](CO)C(=O)O)ccc2Cl)cccc1-c1ccccc1 ; Cc1ccc(CN[C@H](CO)C(=O)O)cc1/C=C/c1cccc(-c2ccccc2)c1C]

1.8 Method 1, wherein the compound is an aromatic acetylene or vinyl aromatic compound of the general formula II, in free or pharmaceutically acceptable salt form:

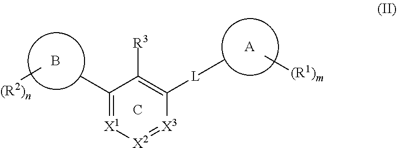

wherein, ring A and ring B are independently an aromatic ring or a heteroaromatic ring;
L is alkynyl, -C(R⁴)═C(R⁵)- or C₂-C₁₀ heteroaryl having at least one N;
X¹ is N or -CR⁶;
X² is N or -CR⁷;
X³ is N or -CR⁸;
X¹, X² and X³ are not N simultaneously;
each of R¹ is independently hydrogen, deuterium, substituted or unsubstituted hydroxy, substituted or unsubstituted amino, halogen, substituted or unsubstituted alkyl or substituted or unsubstituted alkoxy; each of R² is independently hydrogen, deuterium, substituted or unsubstituted hydroxy, substituted or unsubstituted amino, halogen, substituted or unsubstituted alkyl or substituted or unsubstituted alkoxy,

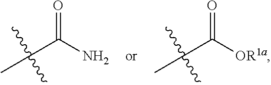

wherein R^{1a} is C₁-C₄ alkyl; or two adjacent R² together with the two atoms on the ring B to which they are attached form a 5-7 membered substituted or unsubstituted carbocycle, or substituted or unsubstituted heterocycle; in the heterocycle, heteroatom is oxygen and/or nitrogen, the number of the heteroatom(s) is 1-4;
R³ is deuterium, halogen, cyano, or substituted or unsubstituted alkyl;
R⁴ and R⁵ are each independently hydrogen, deuterium, substituted or unsubstituted alkyl, substituted or unsubstituted cycloalkyl, or R⁴ and R⁵ together with the carbon-carbon double bond to which they are attached form a 5-7 membered substituted or unsubstituted carbocycle, substituted or unsubstituted heterocycle; in the heterocycle, heteroatom is oxygen and/or nitrogen, the number of the heteroatom(s) is 1-4;
R⁶, R⁷ and R⁸ are each independently hydrogen, deuterium, substituted or unsubstituted hydroxy, halogen, substituted or unsubstituted amino, substituted or unsubstituted alkyl, or substituted or unsubstituted alkoxy, or R⁶ and R⁷ together with the two atoms on the ring C to which they are attached form a 5-7 membered substituted or unsubstituted heterocycle; or R⁷ and R⁸ together with the two atoms on the ring C to which they are attached form a 5-7 membered substituted or unsubstituted heterocycle, in the heterocycle, heteroatom is oxygen and/or nitrogen, the number of the heteroatom(s) is 1-4;
m is 1, 2 or 3;
n is 1 or 2;
in the definition of each R¹, the substituent in the substituted alkyl or the substituted alkoxy is selected from the group consisting of halogen, C₁-C₄ alkyl, hydroxy,

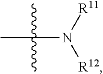

benzyl, benzyl substituted by cyano, C₁-C₄ alkoxy, C₁-C₄ carboxyl, C₁-C₄ ester group or C₁-C₄ acylamino; the substituent in the substituted hydroxy or the substituted amino is selected from the group consisting of C₁-C₄ alkyl, benzyl, benzyl substituted by cyano, C₁-C₄ alkoxy, C₁-C₄ carboxyl, C₁-C₄ ester group or C₁-C₄ acylamino;
in the definition of each R², the substituent in the substituted alkyl or the substituted alkoxy is selected from the group consisting of halogen, C₁-C₄ alkyl hydroxy,

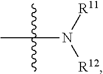

C₁-C₄ alkoxy, C₁-C₄ carboxyl, C₁-C₄ ester group or C₁-C₄ acylamino; the substituent in the substituted hydroxy or the substituted amino is selected from the group consisting of C₁-C₄ alkyl, benzyl, benzyl substituted by cyano, C₁-C₄ alkoxy, C₁-C₄ carboxyl, C₁-C₄ ester group or C₁-C₄ acylamino; when two adjacent R² together with the two atoms on the ring B to which they are attached form a 5-7 membered substituted carbocycle or substituted heterocycle, the substituent in the substituted carbocycle or in the substituted heterocycle is selected from the group consisting of halogen, C₁-C₄ alkyl, hydroxy,

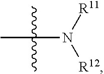

C₁-C₄ alkoxy, C₁-C₄ carboxyl, C₁-C₄ ester group or C₁-C₄ acylamino, when there are more substituents than one, the substituents are the same or different;
in the definition of R⁴ or R⁵, the substituent in the substituted alkyl or the substituted cycloalkyl is selected from the group consisting of halogen, C₁-C₄ alkyl, hydroxy, amino, C₁-C₄ alkoxy, C₁-C₄ carboxyl, C₁-C₄ ester group or C₁-C₄ acylamino; where R⁴ and R⁵ together with the carbon-carbon double bond to which they are attached form a 5-7 membered substituted carbocycle, or, substituted heterocycle, the substituent in the substituted carbocycle or the substituted heterocycle is selected from the group consisting of halogen, C₁-C₄ alkyl, hydroxy,

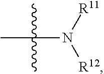

C₁-C₄ carboxyl, C₁-C₄ ester group or C₁-C₄ acylamino; when there are more substituents than one, the substituents are the same or different;
in the definition of R⁶, R⁷ or R⁸, the substituent in the substituted alkyl or the substituted alkoxy is selected from the group consisting of halogen, C₁-C₄ alkyl, hydroxy,

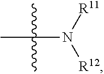

C₁-C₄ alkoxy, C₁-C₄ carboxyl, C₁-C₄ ester group or C₁-C₄ acylamino; the substituent in the substituted hydroxy or the substituted amino is selected from the group consisting of C₁-C₄ alkyl, benzyl, benzyl substituted by cyano, C₁-C₄ alkoxy, C₁-C₄ carboxyl, C₁-C₄ ester group or C₁-C₄ acylamino; wherein R⁶ and R⁷ together with the two atoms on the ring C to which they are attached form a 5-7 membered substituted heterocycle, or when R⁷ and R⁸ together with the two atoms on the ring C to which they are attached form a 5-7 membered substituted heterocycle, the substituent in the substituted heterocycle is selected from the group consisting of halogen, C₁-C₄ alkyl, hydroxy,

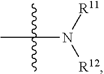

C₁-C₄ alkoxy, C₁-C₄ ester group or C₁-C₄ acylamino; when there are more substituents than one, the substituents are the same or different;
in

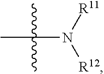

R¹¹ and R¹² are independently hydrogen, substituted or unsubstituted alkyl, alkoxy, hydroxyalkyl, aminoalkyl, substituted or unsubstituted C₆-C₁₄ aryl or substituted or unsubstituted C₃-C₆ cycloalkyl; or R¹¹ and R¹² together with the nitrogen atom to which they are attached form a 5-7 membered substituted or unsubstituted heterocycle; in the heterocycle, the heteroatom is nitrogen, or nitrogen and oxygen, the number of the heteroatom(s) is 1-4;
in the definition of R¹¹ and R¹² , the substituent in the substituted alkyl, the substituted C₆-C₁₄ aryl or the substituted C₃-C₆ cycloalkyl is selected from the group consisting of halogen, C₁-C₄ alkyl, hydroxy,

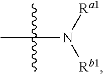

C₁-C₄ alkoxy, C₁-C₄ carboxyl, C₁-C₄ ester group or C₁-C₄ acylamino; when R¹¹ and R¹² together with the nitrogen atom to which they are attached form a 5-7 membered substituted or unsubstituted heterocycle, the substituent in the substituted heterocycle is selected from the group consisting of halogen, C₁-C₄ alkyl, substituted C₁-C₄ alkyl, hydroxy.

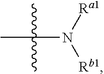

C₁-C₄ alkoxy, C₁-C₄ carboxyl, C₁-C₄ ester group or C₁-C₄ acylamino; the substituent in the substituted C₁-C₄ alkyl is selected from the group consisting of halogen, C₁-C₄ alkyl, hydroxy,

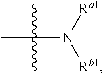

C₁-C₄ alkoxy, C₁-C₄ carboxyl, C₁-C₄ ester group or C₁-C₄ acylamino; when they are more substituents than one, the substituents a the same or different; in

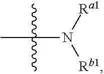

R^{a1} and R^{b1} are independently hydrogen, C₁-C₄ alkyl or

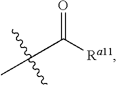

R^{a11} is C₁-C₄ alkyl.
1.9 Method 1 or Method 1.1, wherein the compound is selected from a group consisting of:

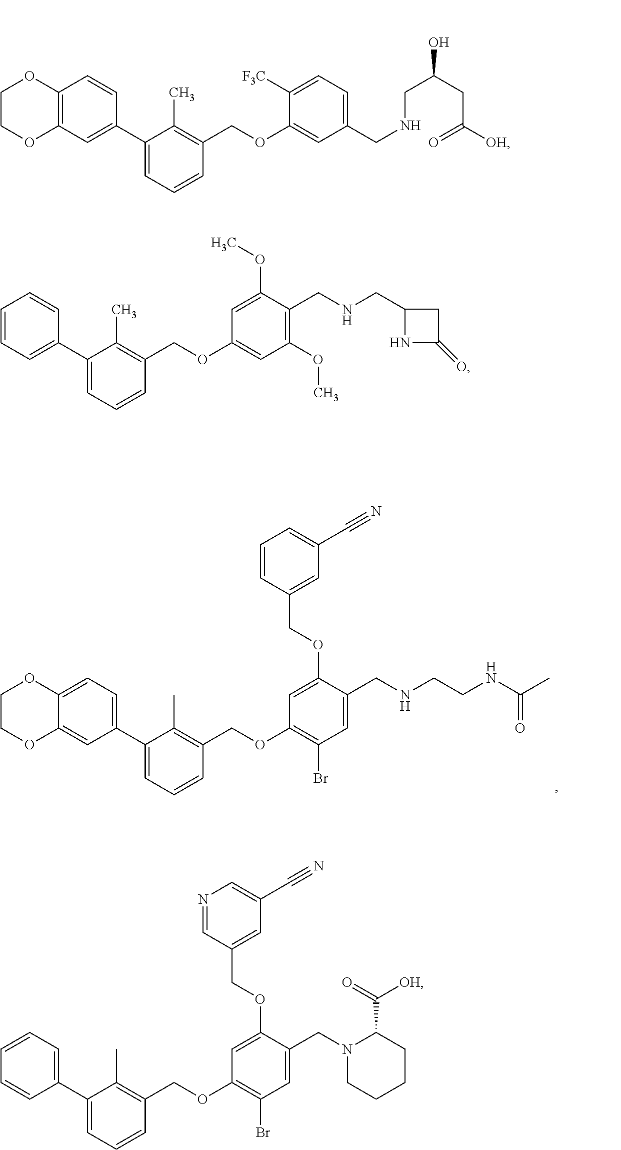 [SMILES: Cc1c(COc2cc(CNC[C@@H](O)CC(=O)O)ccc2C(F)(F)F)cccc1-c1ccc2c(c1)OCCO2 ; COc1cc(OCc2cccc(-c3ccccc3)c2C)cc(OC)c1CNCC1CC(=O)N1 ; CC(=O)NCCNCc1cc(Br)c(OCc2cccc(-c3ccc4c(c3)OCCO4)c2C)cc1OCc1cccc(C#N)c1 ; Cc1c(COc2cc(OCc3cncc(C#N)c3)c(CN3CCCC[C@H]3C(=O)O)cc2Br)cccc1-c1ccccc1]

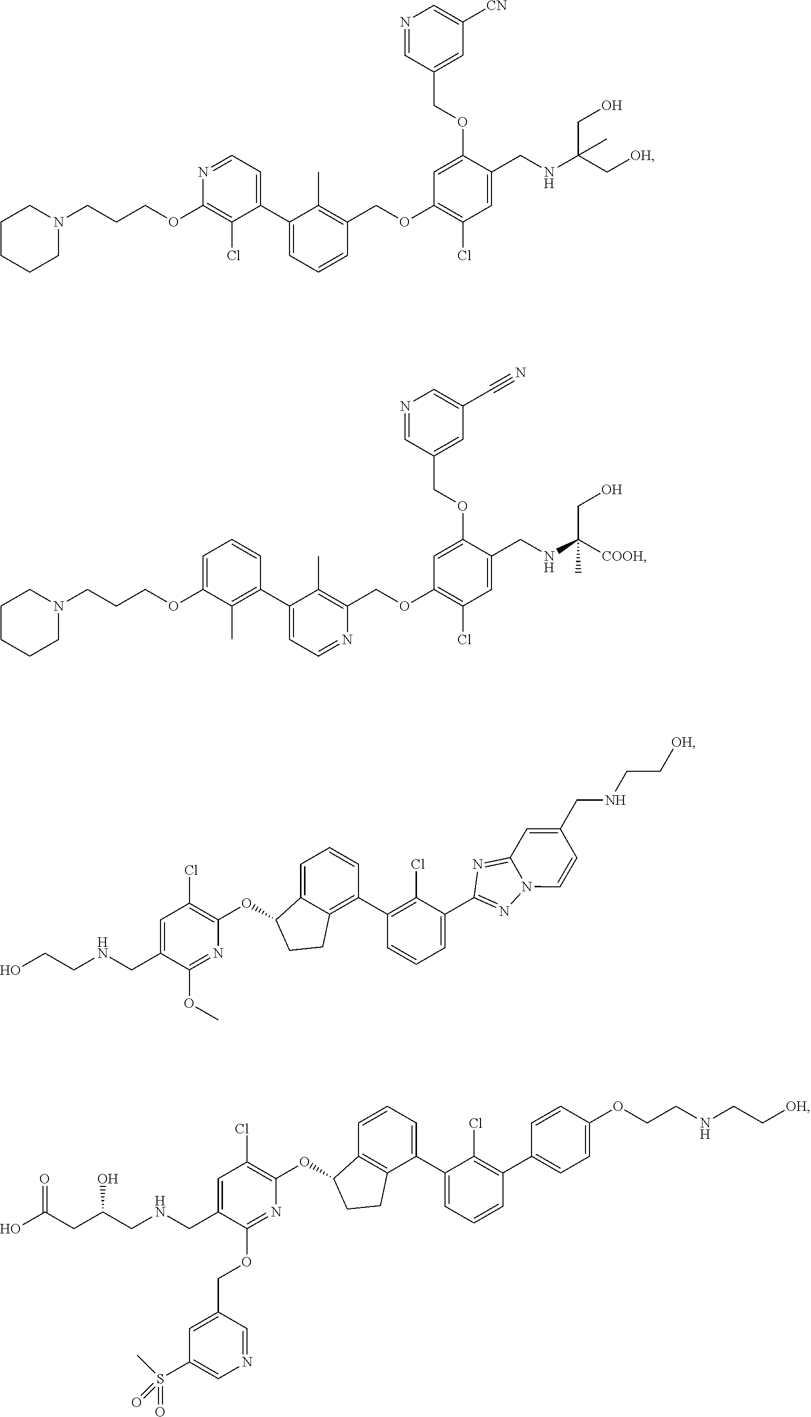 [SMILES: Cc1c(COc2cc(OCc3cncc(C#N)c3)c(CNC(C)(CO)CO)cc2Cl)cccc1-c1ccnc(OCCCN2CCCCC2)c1Cl ; Cc1c(OCCCN2CCCCC2)cccc1-c1ccnc(COc2cc(OCc3cncc(C#N)c3)c(CN[C@@](C)(CO)C(=O)O)cc2Cl)c1C ; COc1nc(O[C@H]2CCc3c(-c4cccc(-c5nc6cc(CNCCO)ccn6n5)c4Cl)cccc32)c(Cl)cc1CNCCO ; CS(=O)(=O)c1cncc(COc2nc(O[C@H]3CCc4c(-c5cccc(-c6ccc(OCCNCCO)cc6)c5Cl)cccc43)c(Cl)cc2CNC[C@@H](O)CC(=O)O)c1]

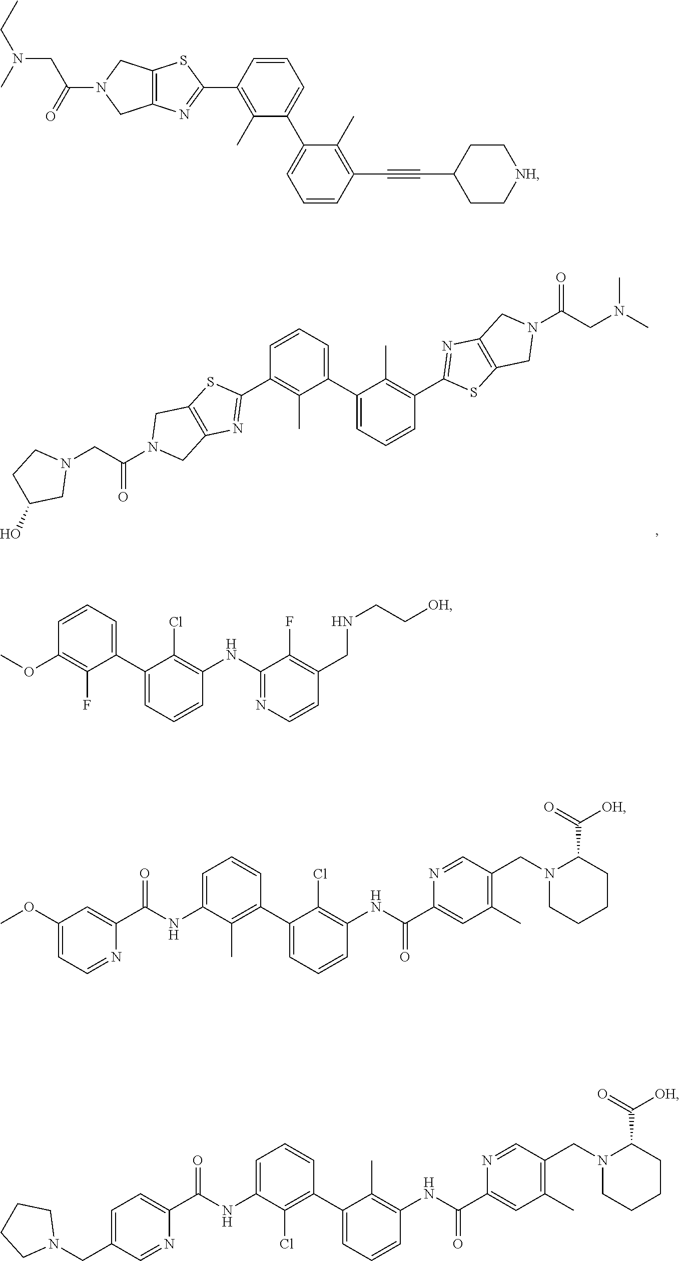

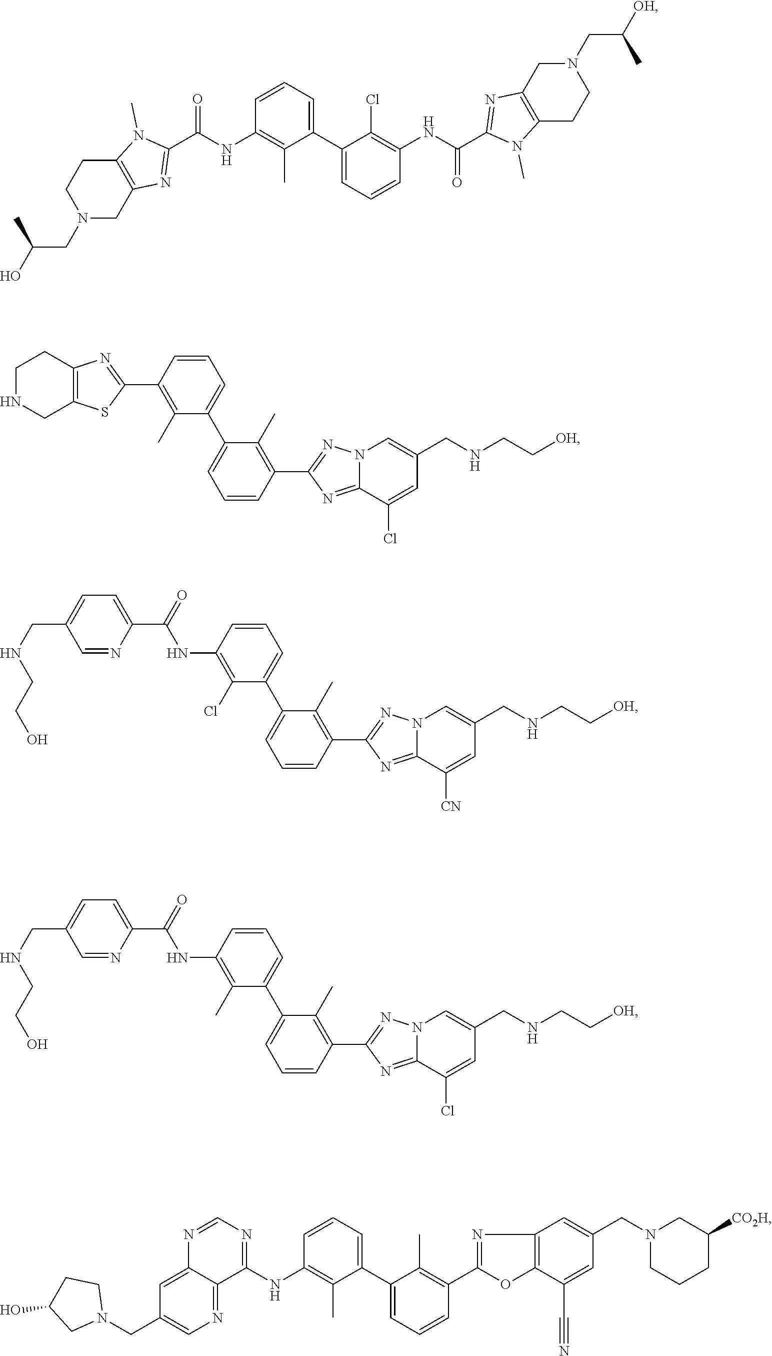 [SMILES: Cc1c(NC(=O)c2nc3c(n2C)CCN(C[C@H](C)O)C3)cccc1-c1cccc(NC(=O)c2nc3c(n2C)CCN(C[C@H](C)O)C3)c1Cl ; Cc1c(-c2nc3c(Cl)cc(CNCCO)cn3n2)cccc1-c1cccc(-c2nc3c(s2)CNCC3)c1C ; Cc1c(-c2nc3c(C#N)cc(CNCCO)cn3n2)cccc1-c1cccc(NC(=O)c2ccc(CNCCO)cn2)c1Cl ; Cc1c(NC(=O)c2ccc(CNCCO)cn2)cccc1-c1cccc(-c2nc3c(Cl)cc(CNCCO)cn3n2)c1C ; Cc1c(Nc2ncnc3cc(CN4CC[C@@H](O)C4)cnc23)cccc1-c1cccc(-c2nc3cc(CN4CCC[C@H](C(=O)O)C4)cc(C#N)c3o2)c1C]

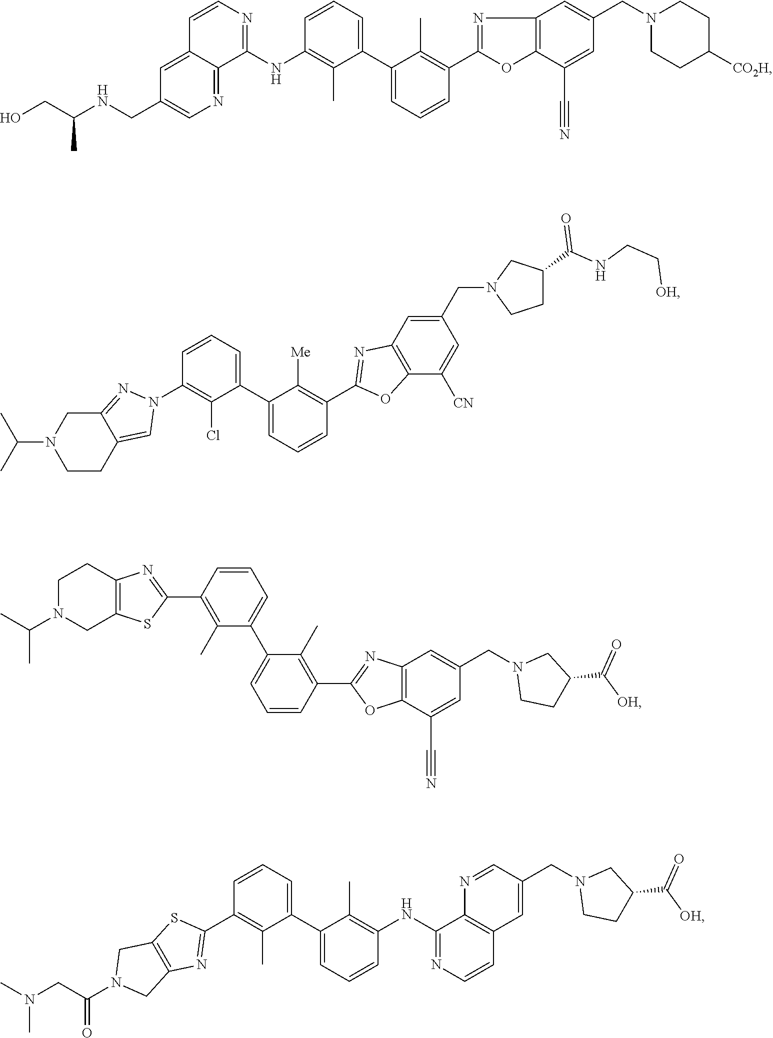 [SMILES: Cc1c(Nc2nccc3cc(CN[C@@H](C)CO)cnc23)cccc1-c1cccc(-c2nc3cc(CN4CCC(C(=O)O)CC4)cc(C#N)c3o2)c1C ; Cc1c(-c2nc3cc(CN4CC[C@@H](C(=O)NCCO)C4)cc(C#N)c3o2)cccc1-c1cccc(-n2cc3c(n2)CN(C(C)C)CC3)c1Cl ; Cc1c(-c2nc3cc(CN4CC[C@@H](C(=O)O)C4)cc(C#N)c3o2)cccc1-c1cccc(-c2nc3c(s2)CN(C(C)C)CC3)c1C ; Cc1c(Nc2nccc3cc(CN4CC[C@@H](C(=O)O)C4)cnc23)cccc1-c1cccc(-c2nc3c(s2)CN(C(=O)CN(C)C)C3)c1C]

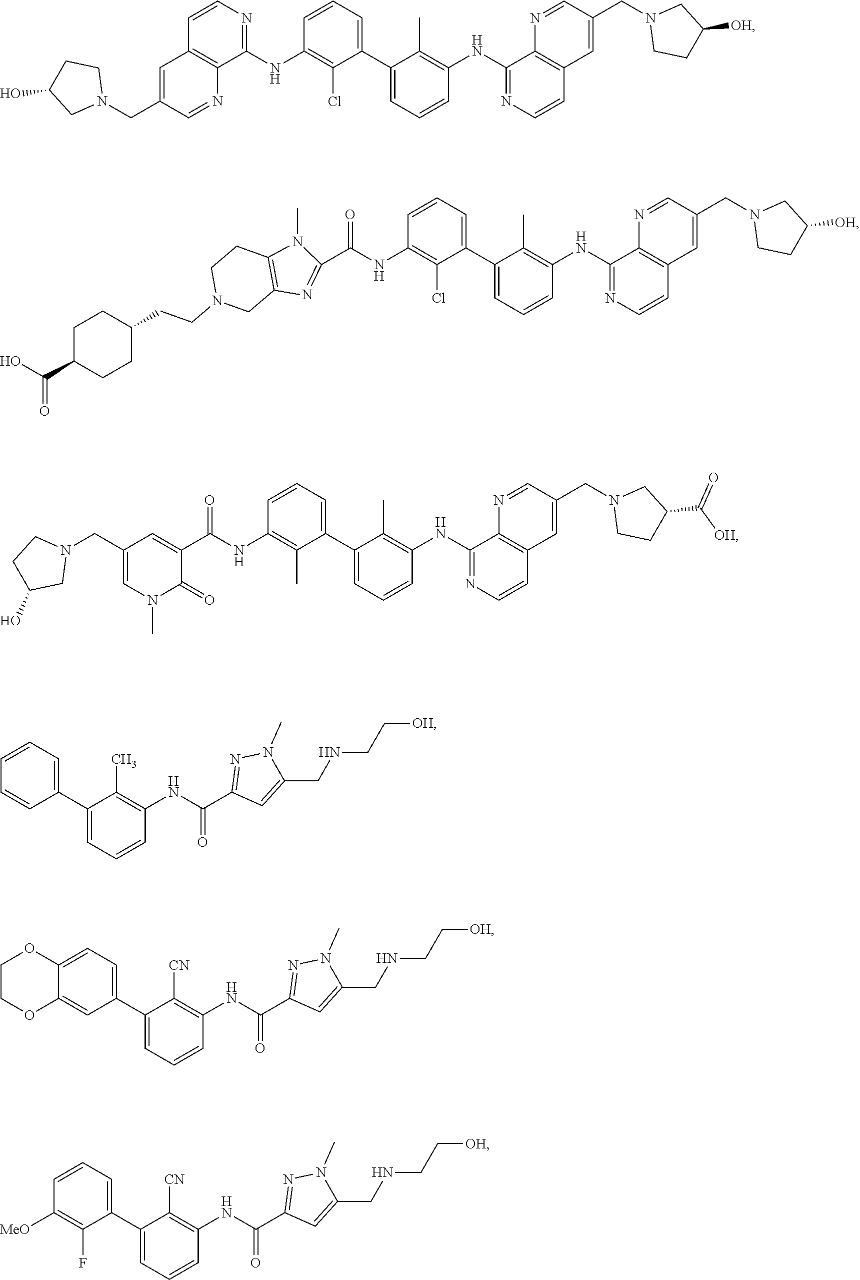 [SMILES: Cc1c(Nc2nccc3cc(CN4CC[C@H](O)C4)cnc23)cccc1-c1cccc(Nc2nccc3cc(CN4CC[C@@H](O)C4)cnc23)c1Cl ; Cc1c(Nc2nccc3cc(CN4CC[C@@H](O)C4)cnc23)cccc1-c1cccc(NC(=O)c2nc3c(n2C)CCN(CC[C@H]2CC[C@H](C(=O)O)CC2)C3)c1Cl ; Cc1c(NC(=O)c2cc(CN3CC[C@@H](O)C3)cn(C)c2=O)cccc1-c1cccc(Nc2nccc3cc(CN4CC[C@@H](C(=O)O)C4)cnc23)c1C ; Cc1c(NC(=O)c2cc(CNCCO)n(C)n2)cccc1-c1ccccc1 ; Cn1nc(C(=O)Nc2cccc(-c3ccc4c(c3)OCCO4)c2C#N)cc1CNCCO ; COc1cccc(-c2cccc(NC(=O)c3cc(CNCCO)n(C)n3)c2C#N)c1F]

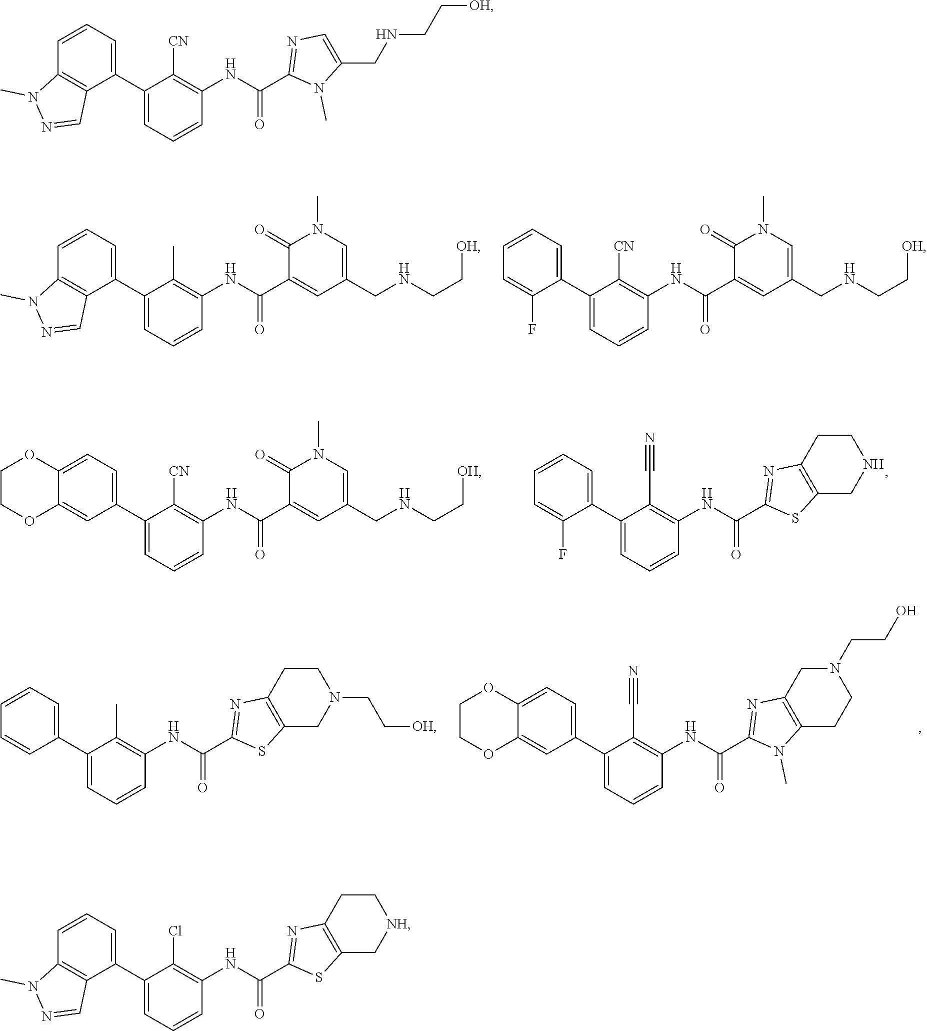

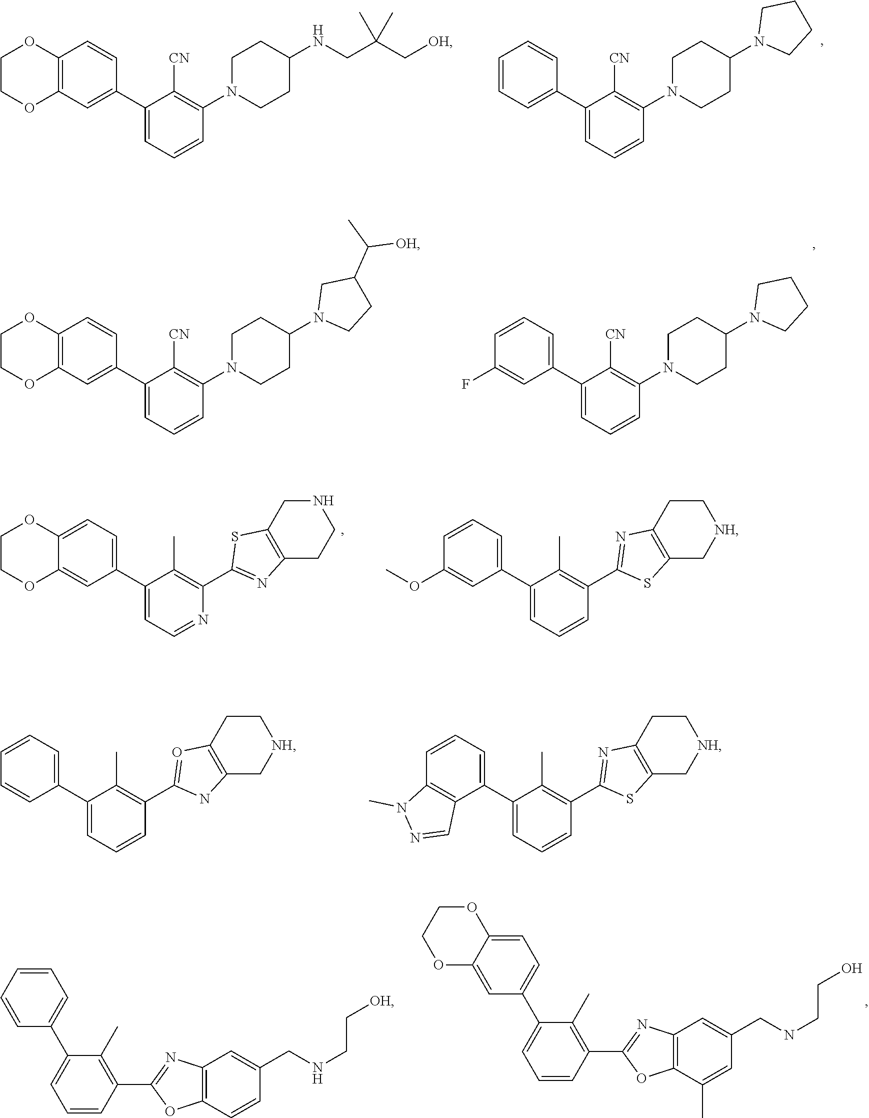

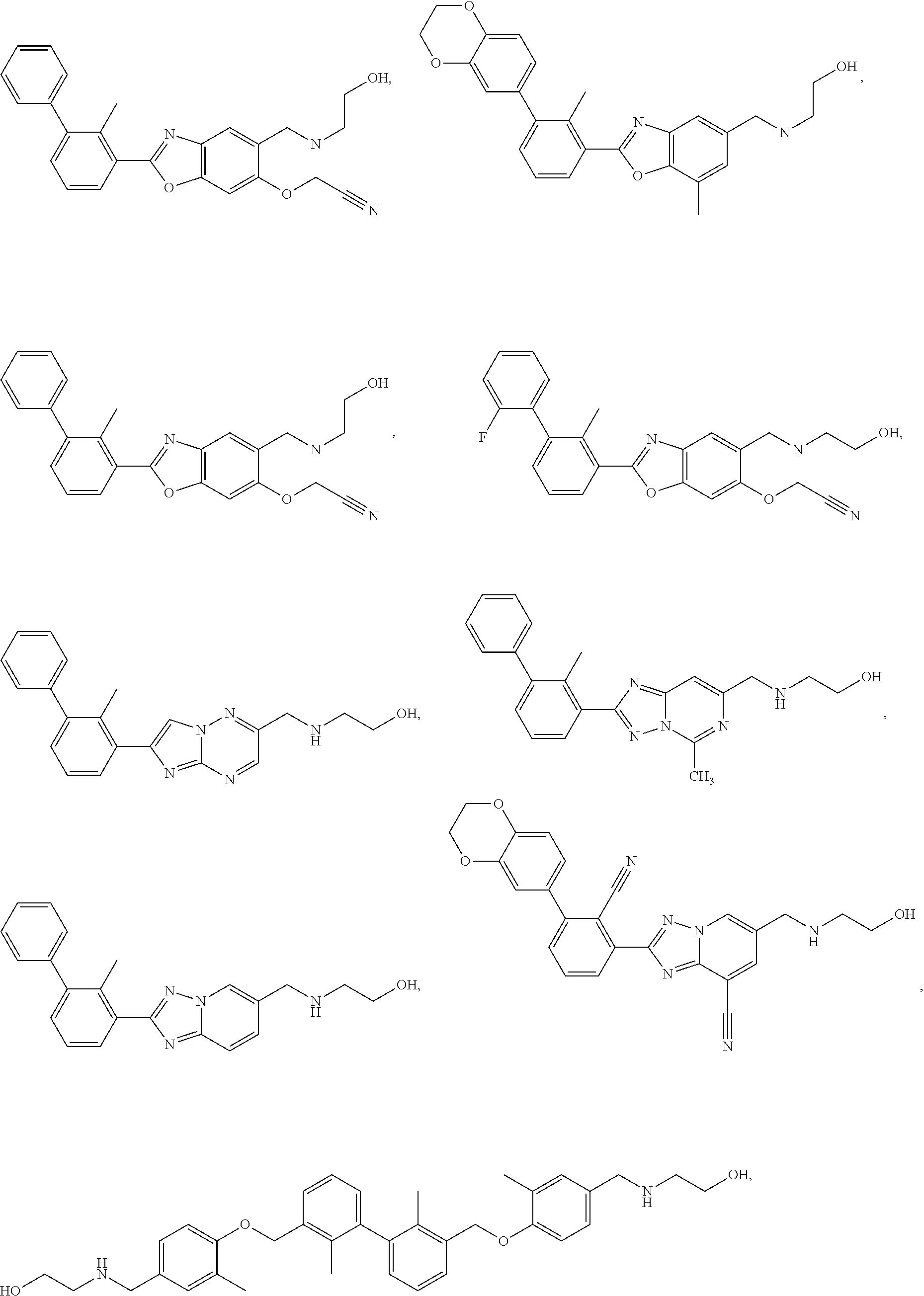

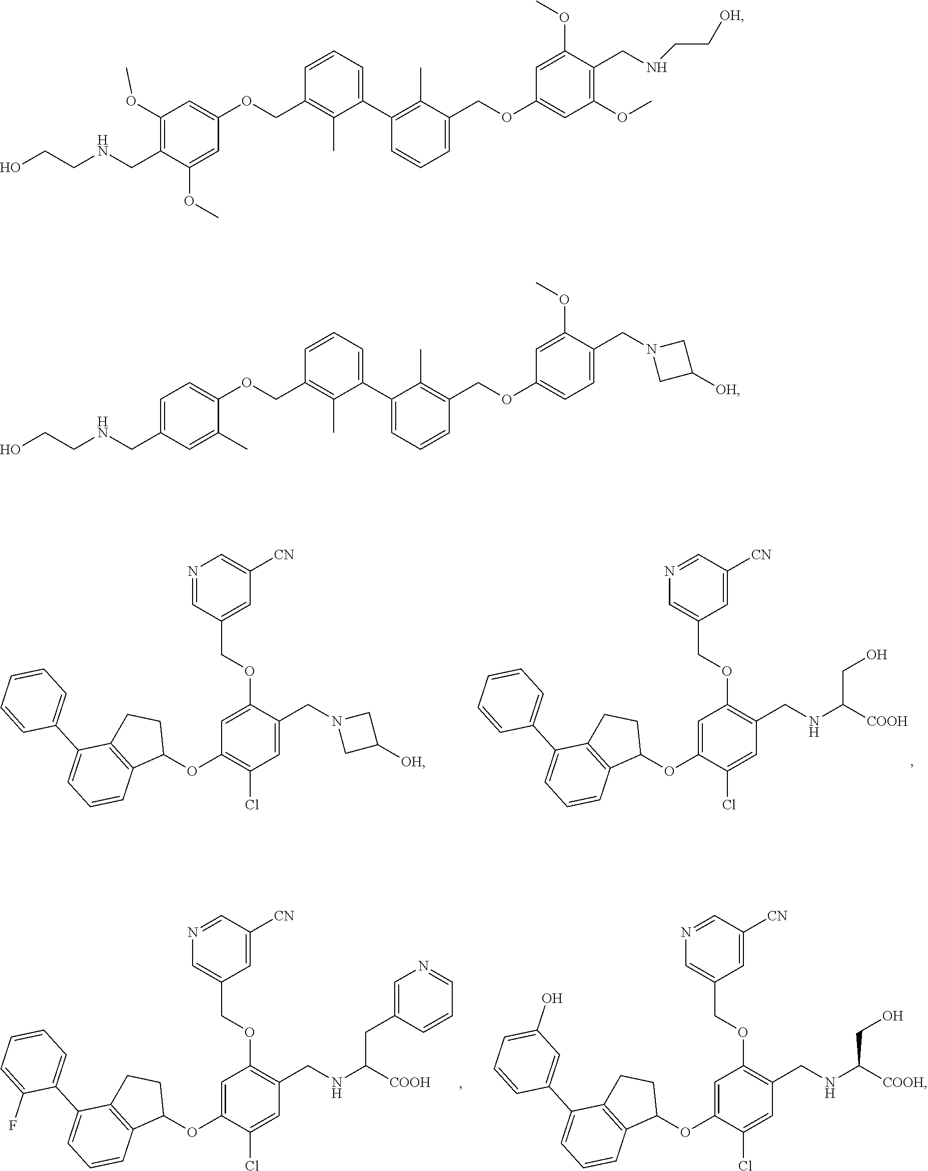 [SMILES: COc1cc(OCc2cccc(-c3cccc(COc4cc(OC)c(CNCCO)c(OC)c4)c3C)c2C)cc(OC)c1CNCCO ; COc1cc(OCc2cccc(-c3cccc(COc4ccc(CNCCO)cc4C)c3C)c2C)ccc1CN1CC(O)C1 ; N#Cc1cncc(COc2cc(OC3CCc4c(-c5ccccc5)cccc43)c(Cl)cc2CN2CC(O)C2)c1 ; N#Cc1cncc(COc2cc(OC3CCc4c(-c5ccccc5)cccc43)c(Cl)cc2CNC(CO)C(=O)O)c1 ; N#Cc1cncc(COc2cc(OC3CCc4c(-c5ccccc5F)cccc43)c(Cl)cc2CNC(Cc2cccnc2)C(=O)O)c1 ; N#Cc1cncc(COc2cc(OC3CCc4c(-c5cccc(O)c5)cccc43)c(Cl)cc2CN[C@@H](CO)C(=O)O)c1]

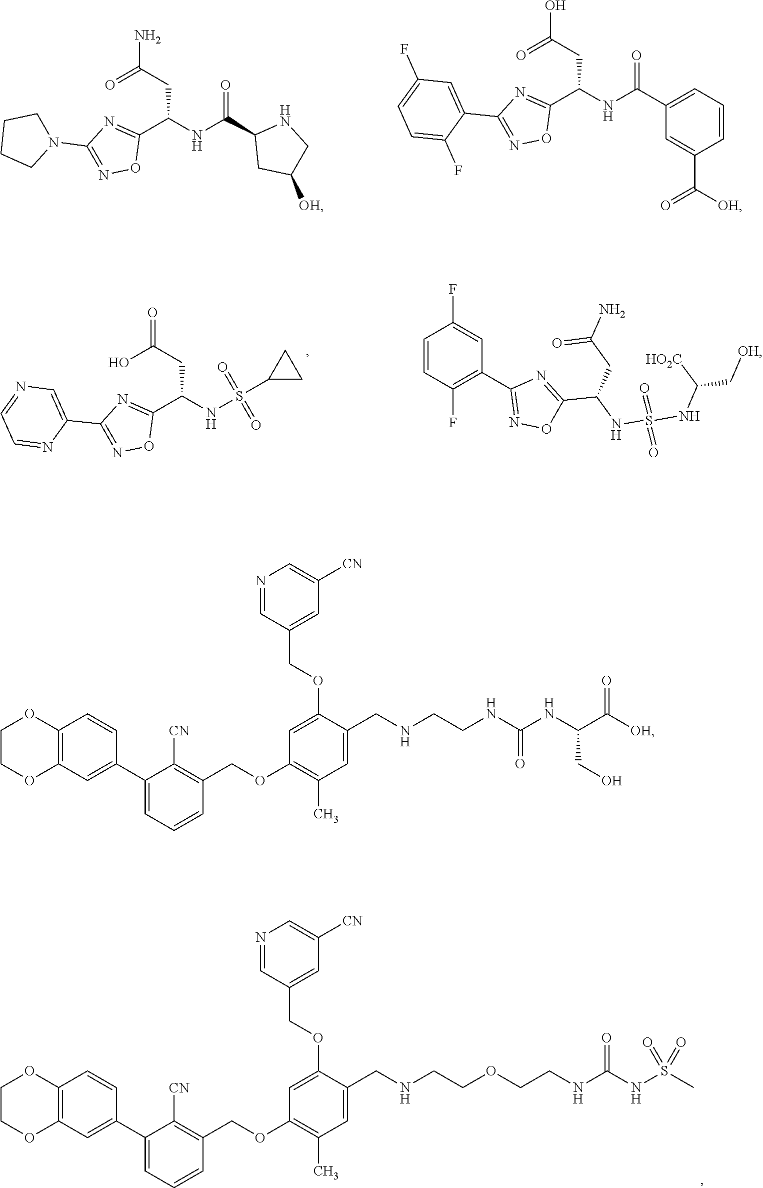 [SMILES: NC(=O)C[C@H](NC(=O)[C@@H]1C[C@H](O)CN1)c1nc(N2CCCC2)no1 ; O=C(O)C[C@H](NC(=O)c1cccc(C(=O)O)c1)c1nc(-c2cc(F)ccc2F)no1 ; O=C(O)C[C@H](NS(=O)(=O)C1CC1)c1nc(-c2cnccn2)no1 ; NC(=O)C[C@H](NS(=O)(=O)N[C@@H](CO)C(=O)O)c1nc(-c2cc(F)ccc2F)no1 ; Cc1cc(CNCCNC(=O)N[C@@H](CO)C(=O)O)c(OCc2cncc(C#N)c2)cc1OCc1cccc(-c2ccc3c(c2)OCCO3)c1C#N ; Cc1cc(CNCCOCCNC(=O)NS(C)(=O)=O)c(OCc2cncc(C#N)c2)cc1OCc1cccc(-c2ccc3c(c2)OCCO3)c1C#N]

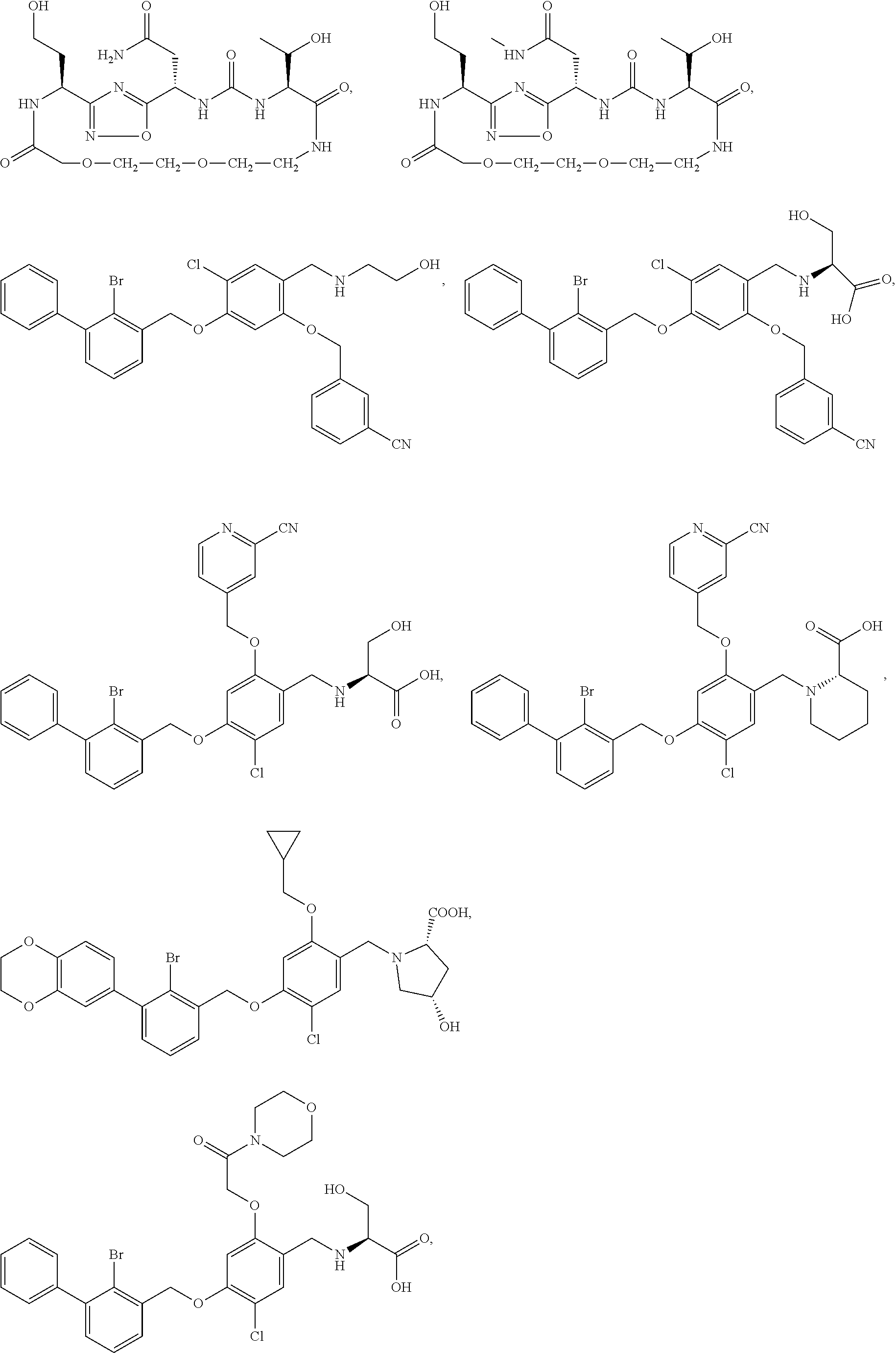

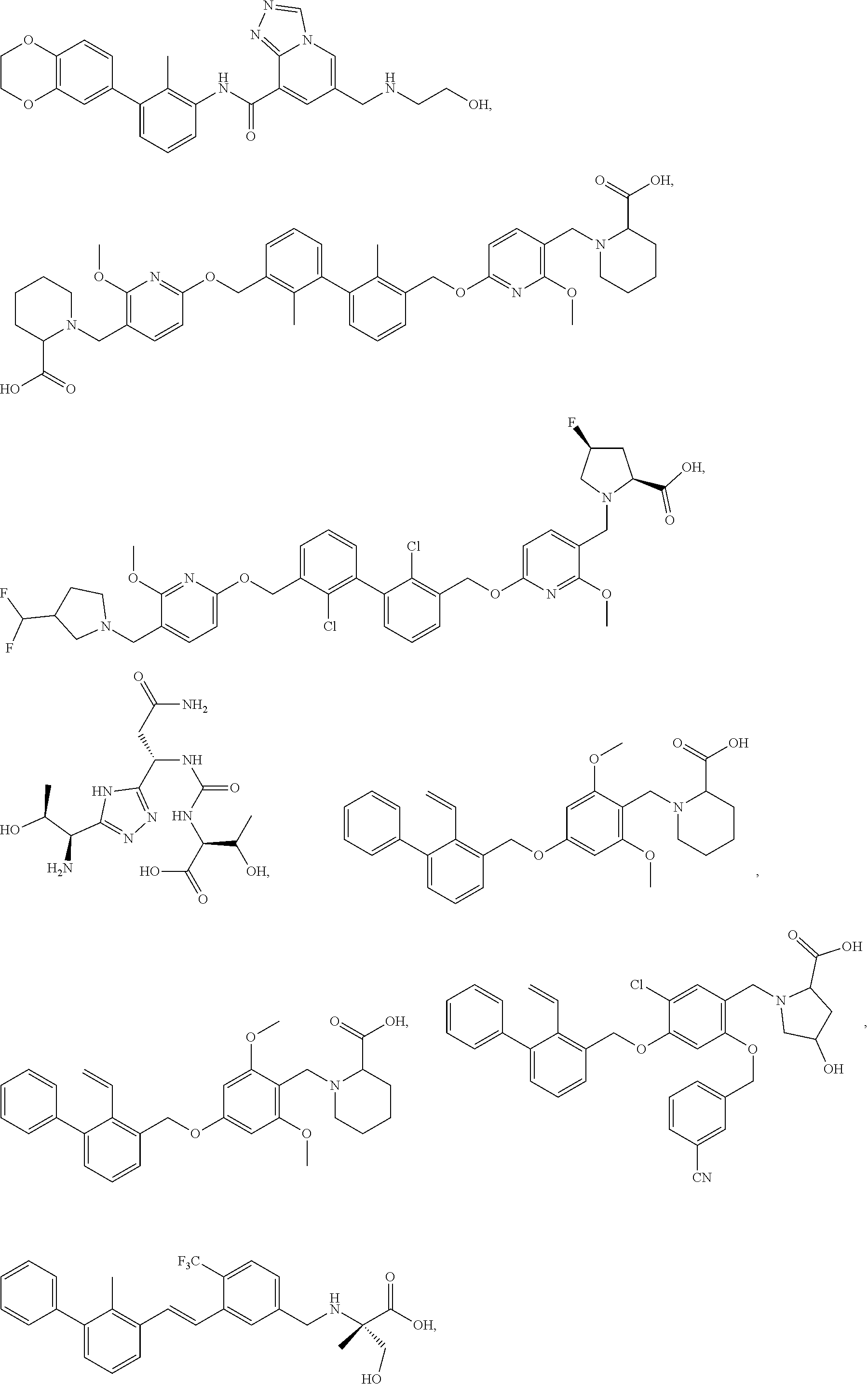

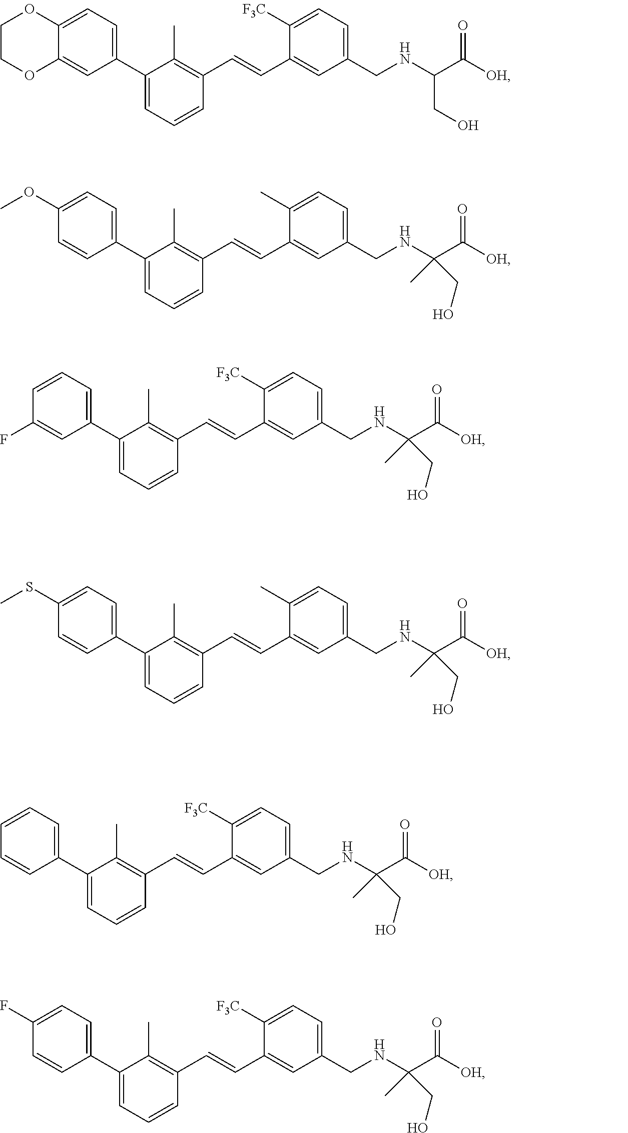 [SMILES: Cc1c(/C=C/c2cc(CNC(CO)C(=O)O)ccc2C(F)(F)F)cccc1-c1ccc2c(c1)OCCO2 ; COc1ccc(-c2cccc(/C=C/c3cc(CNC(C)(CO)C(=O)O)ccc3C)c2C)cc1 ; Cc1c(/C=C/c2cc(CNC(C)(CO)C(=O)O)ccc2C(F)(F)F)cccc1-c1cccc(F)c1 ; CSc1ccc(-c2cccc(/C=C/c3cc(CNC(C)(CO)C(=O)O)ccc3C)c2C)cc1 ; Cc1c(/C=C/c2cc(CNC(C)(CO)C(=O)O)ccc2C(F)(F)F)cccc1-c1ccccc1 ; Cc1c(/C=C/c2cc(CNC(C)(CO)C(=O)O)ccc2C(F)(F)F)cccc1-c1ccc(F)cc1]

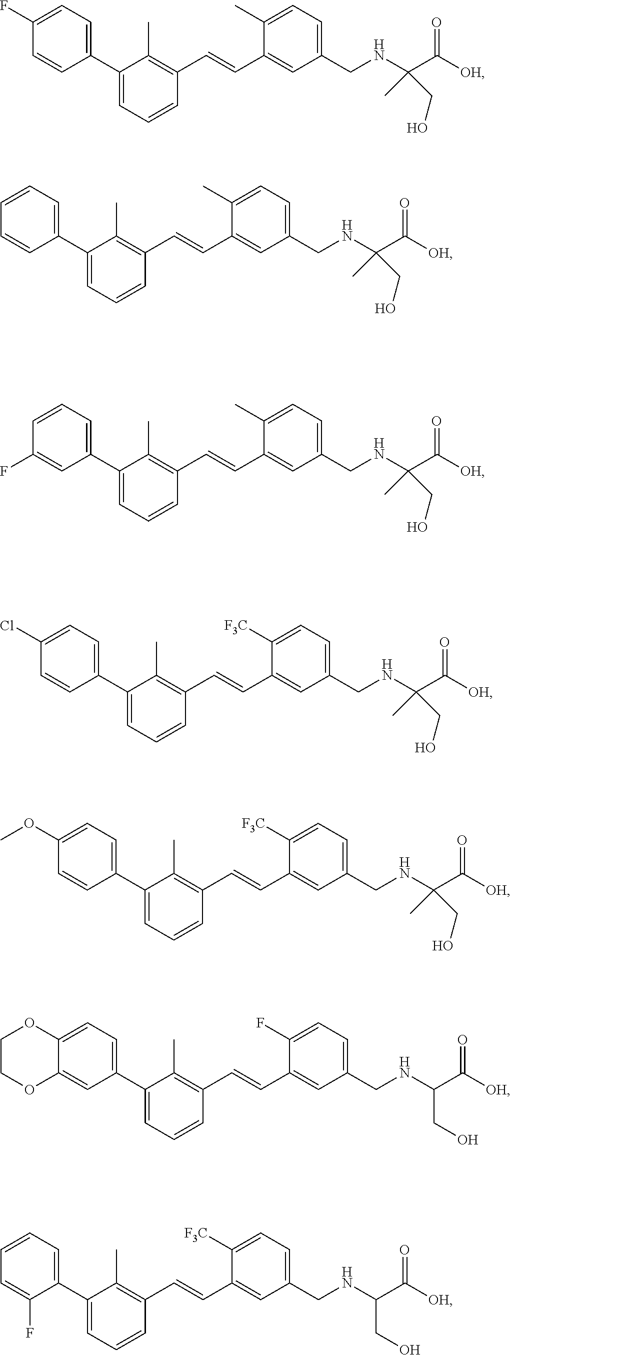

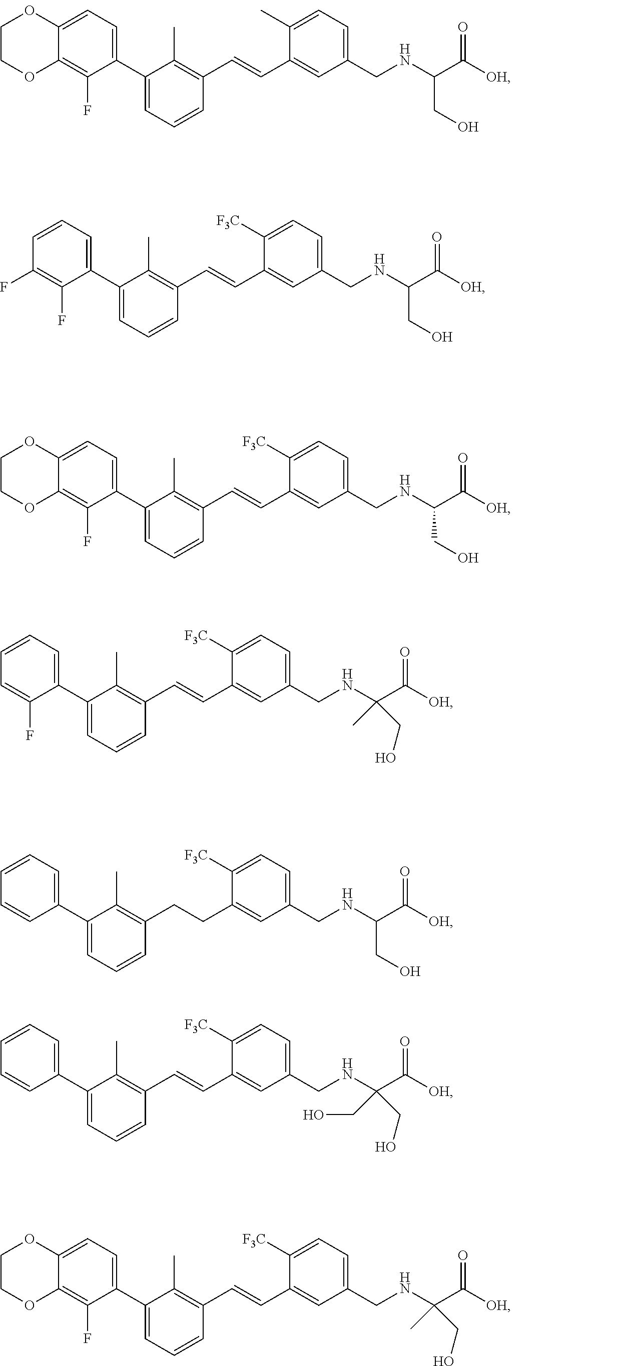

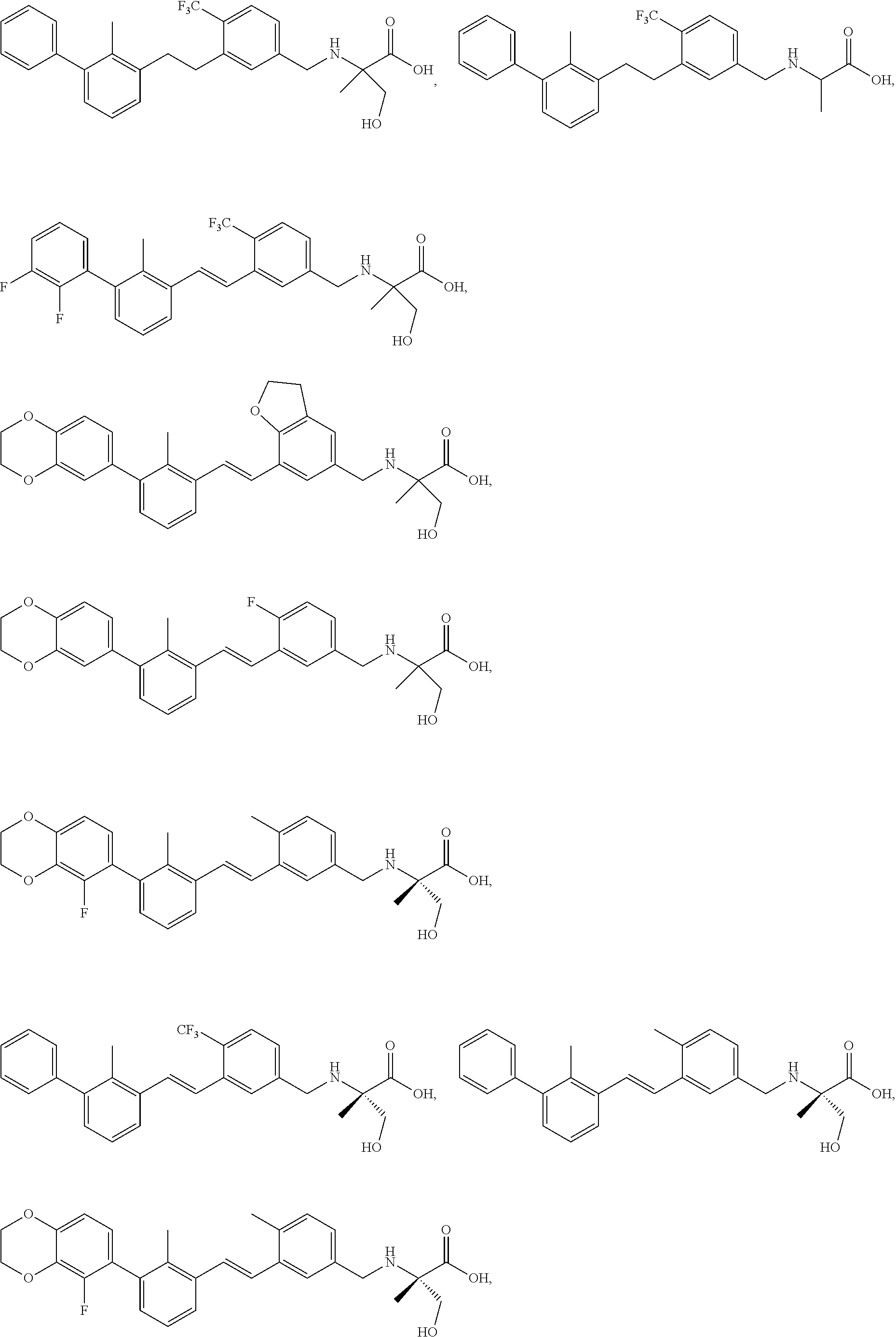

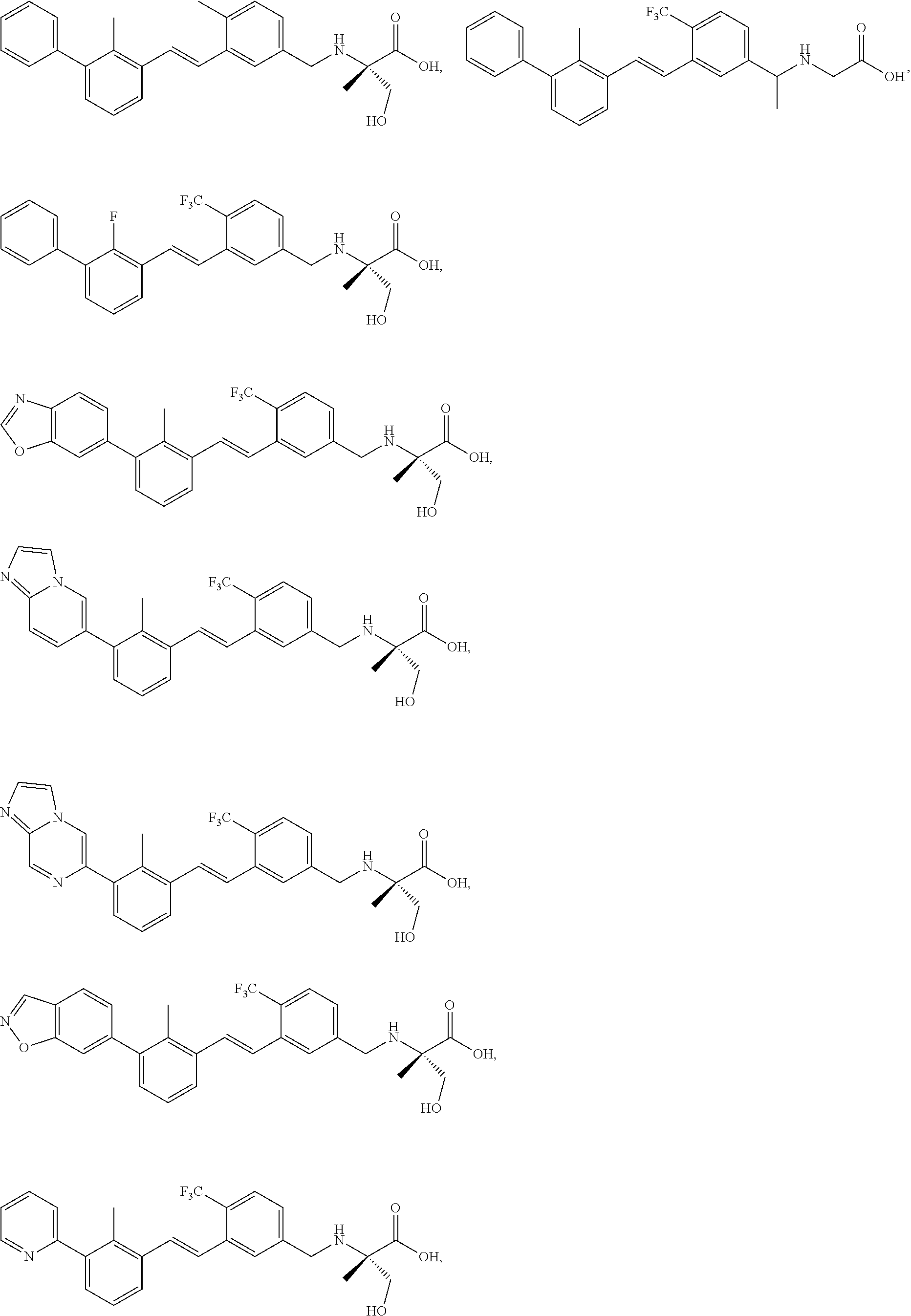

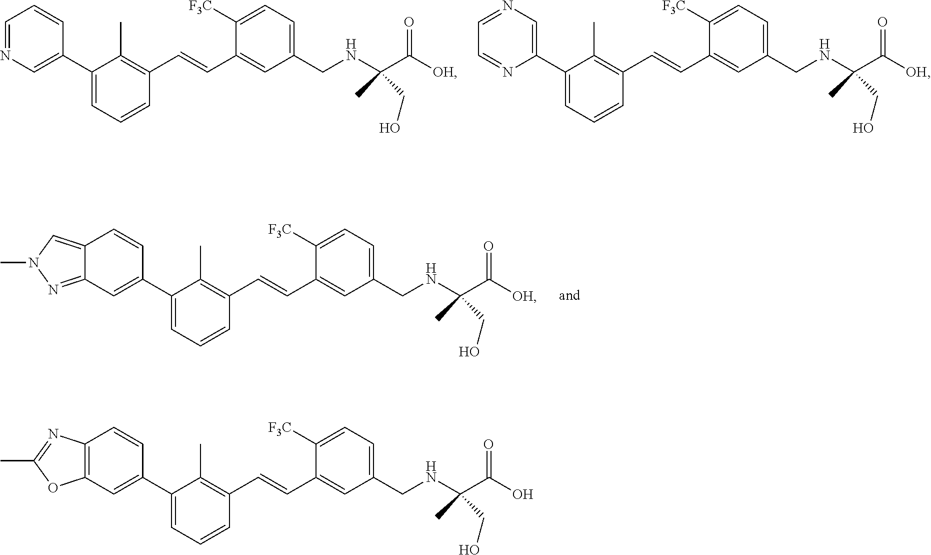

in free or pharmaceutically acceptable salt form.
1.10 Any of Methods 1-1.9, wherein the compound is

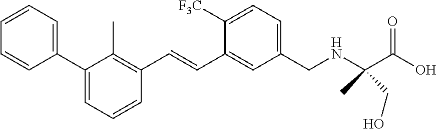 [SMILES: Cc1c(/C=C/c2cc(CN[C@@](C)(CO)C(=O)O)ccc2C(F)(F)F)cccc1-c1ccccc1]

in free or pharmaceutically acceptable salt form.
1.11 Any of Methods 1-1.10, wherein the cancer is bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or combinations of the cancers.
1.12 Any of Methods 1-1.11, wherein the cancer is breast cancer.
1.13 Any of Methods 1-1.11, wherein the cancer is melanomas,
1.14 Any of Methods 1-1.11, wherein the cancer is colorectal cancer.
1.15 Any of Methods 1-1.14, wherein the subject is a human.
1.16 Any of Methods 1-1.15, wherein the compound is administered orally.
1.17 Any of Methods 1-1.16, wherein the compound is administered at a total dose of 20-300 mg/kg, 30-240 mg/kg, 40-200 mg/kg, 50-190 mg/kg, 60-180 mg/kg, 70-170 mg/kg, 80-160 mg/kg, 90-150 mg/kg or 100-140 mg/kg per day.
1.18 Any of Methods 1-1.17, wherein the compound is administered at an amount of about 10-150 mg/kg, 15-120 mg/kg, 20-100 mg/kg, 30-90 mg/kg, or 40-80 mg/kg body weight twice a day (BID).
1.19 Any of Methods 1-1.18, wherein the compound is administered at an amount of about 30 mg/kg, 35 mg/kg, 40 mg/kg, 45 mg/kg, 50 mg/kg, 55 mg/kg, 60 mg/kg, 65 mg/kg, 70 mg/kg, 75, mg/kg. 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg or 150 mg/kg body weight BID,
1.20 Any of Methods 1-1.19, wherein the compound is administered at an amount of about 30 mg/kg body weight BID.
1.21 Any of Methods 1-1.19, wherein the compound is administered at an amount of about 60 mg/kg body weight BID.
1.22 Any of Methods 1-1.19, wherein the compound is administered at an amount of about 90 mg/kg body weight BID.
1.23 Any of Methods 1-1.19, wherein the compound is administered at an amount of about 120 mg/kg body weight BID.
1.24 Any of Methods 1-1.23, wherein the subject has previously received cancer treatment.
1.25 Method 1.24, wherein the cancer treatment is chemotherapy.
1.26 Method 1.25, wherein the chemotherapy comprises a platinum containing chemotherapeutic agent.
1.27 Any of Method 1.25 or Method 1.26, wherein the chemotherapy is platinum-containing doublet chemotherapy.
1.28 Method 1.24, wherein the cancer treatment comprises administering an anti-PD-1 antibody to the subject.
1.29 Method 1,28, wherein the anti-PD-1 antibody is pembrolizumab, nivolumab or cemiplimab.
1.30 Method 1.24, wherein the cancer treatment comprises administering an anti-PD-L1 antibody to the subject.
1.31 Method 1.30, wherein the anti-PD-L1 antibody is atezolizumab, durvalumab, or avelumab.
1.32 Any of Methods 1.22-1.31, wherein the subject is not responsive to the cancer treatment.
1.33 Any of Methods 1-1.32, wherein the subject does not have a history of significant autoimmune disease.
1.34 Any of Methods 1-1.33, wherein the subject has not received organ or bone marrow transplants.
1.35 Any of Methods 1-1.34, wherein the subject has IFP of 10-50 mmHg, 15-45 mmHg or 20-40 mmHg.
1.36 Any of Methods 1-1.35, wherein the subject has IFP of at least 15 mmHg, at least 20 mmHg, at least 25 mmHg, at least 30 mmHg, at least 35 mmHg, at least 40 mmHg, at least 45 mmHg or at least 50 mmHg,
1.37 Any of Methods 1-1.36, wherein the IFP is measured by a micropuncture technique, a wick-in-needle technique or MRI technology.
1.38 Any foregoing method further comprising administration of an additional active agent selected from at least one of an antibody binding to PD-1 or PD-L1, an inhibitor of the CTLA-4/B7 interaction, or an inhibitor binding to vascular endothelial growth factor (VEGF).

In another embodiment, the invention provides use of a compound, or a pharmaceutically acceptable salt or prodrug thereof, as defined in any one of Methods 1-1.10 in the manufacture of a medicament for treating a cancer in a subject with a tumor having interstitial fluid pressure (IFP) of at least 10 mmHg, e.g., in any one of Methods 1-1.38.

In another embodiment, the invention provides a compound which is an inhibitor of the interaction between the PD-1 receptor and its ligand PD-L1 and which is not a protein, e.g., a compound as defined in any one of Methods 1.1-1.10, for use in treating a cancer in a subject with a tumor having interstitial fluid pressure (IFP) of at least 10 mmHg, e.g., for use in any one of Methods 1-1.38.

In another aspect, the invention provides a method (Method 2) for treating a cancer in a subject having a tumor with interstitial fluid pressure (IFP) at least 10 mmHg, comprising administering to the subject a therapeutically effective amount of (i) a compound or a pharmaceutically acceptable salt or prodrug thereof, wherein the compound is non-protein inhibitor of the interaction between the PD-1 receptor and its ligand PD-L1, and (ii) an antibody binding to PD-1 or PD-L1:
2.1 Method 2, wherein the compound binds PD-L1.
2.2 Any of Method 2 or 2.1, wherein the compound has a molecular weight (MW) less than
1500 Daltons.
2.3 Any of Methods 2-2.2, wherein the compound has an IC₅₀ of less than 100 nM in a PD-1/PD-L1 binding assay.
2.4 Any of Methods 2-2.3, wherein the compound has the structure as described in any of Methods 1.2-1.10, in free or pharmaceutically acceptable salt form.
2.5 Any of Methods 2-2.4, wherein the compound has the structure of

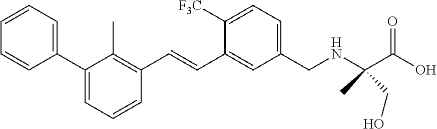 [SMILES: Cc1c(/C=C/c2cc(CN[C@@](C)(CO)C(=O)O)ccc2C(F)(F)F)cccc1-c1ccccc1]

in free or pharmaceutically acceptable salt form.
2.6 Any of Methods 2-2.5, wherein the cancer is bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or combinations of the cancers.
2.7 Any of Methods 2-2.6, wherein the cancer is breast cancer.
2.8 Any of Methods 2-2.6, wherein the cancer is melanomas.
2.9 Any of Methods 2-2.6, wherein the cancer is colorectal cancer.
2.10 Any of Methods 2-2.9, wherein the subject is a human.
2.11 Any of Methods 2-2.10, wherein the compound is simultaneously administered with the antibody.
2.12 Any of Methods 2-2.11, wherein the compound is administered subsequent to the administration of the antibody.
2.13 Any of Methods 2-2.12, wherein the antibody is selected from a group consisting of aembrolizumab, nivolumab, cemiplimab, atezolizumab, durvalumab, and avelumab.
2.14 Any of Methods 2-2.13, wherein the compound is administered orally.
2.15 Any of Methods 2-2.14, wherein the antibody is administered as an intravenous infusion or subcutaneously.
2.16 Any of Methods 2-2.15, wherein the subject has previously received cancer treatment.
2.17 Method 2.16, wherein the cancer treatment is chemotherapy.
2.18 Method 2.17, wherein the chemotherapy comprises a platinum containing chemotherapeutic agent.
2.19 Method 2.17 or 2.18, wherein the chemotherapy is platinum-containing doublet chemotherapy.
2.20 Any of Methods 2-2.19, wherein the subject is not responsive to the cancer treatment.
2.21 Any of Methods 2-2.20, wherein the subject does not have a history of significant autoimmune disease.
2.22 Any of Methods 2-2.21, wherein the subject has not received organ or bone marrow transplants.
2.23 Any of Methods 2-2.22, wherein the compound is administered at a total dose of 20-300 mg/kg, 30-240 mg/kg, 40-200 mg/kg, 50-190 mg/kg, 60-180 mg/kg, 70-170 mg/kg, 80-160 mg/kg, 90-150 mg/kg or 100-140 mg/kg per day.
2.24 Any of Methods 2-2.23, wherein the compound is administered at an amount of about 10-150 mg/kg, 15-120 mg/kg, 20-100 mg/kg, 30-90 mg/kg, or 40-80 mg/kg body weight twice a day (BID).
2.25 Any of Methods 2-2.24, wherein the compound is administered at an amount of about 30 mg/kg, 35 mg/kg, 40 mg/kg, 45 mg/kg, 50 mg/kg, 55 mg/kg, 60 mg/kg, 65 mg/kg, 70 mg/kg, 75, mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg or 150 mg/kg body weight BID.
2.26 Any of Methods 2-2.25, wherein the compound is administered at an amount of about 30 mg/kg body weight BID.
2.27 Any of Methods 2-2.25, wherein the compound is administered at an amount of about 60 mg/kg body weight BID.
2.28 Any of Methods 2-2.25, wherein the compound is administered at an amount of about 90 mg/kg body weight BM.
2.29 Any of Methods 2-2.25, wherein the compound is administered at an amount of about 120 mg/kg body weight BID.
2.30 Any of Methods 2-2.29, wherein the subject has previously received cancer treatment.
2.31 Method 2.30, wherein the cancer treatment is chemotherapy.
2.32 Method 2.31, wherein the chemotherapy comprises a platinum containing chemotherapeutic agent.
2.33 Method 2.31 or Method 2.32, wherein the chemotherapy is platinum-containing doublet chemotherapy.
2.34 Any of Methods 2-2.33, wherein the subject is not responsive to the cancer treatment.
2.35 Any of Methods 2-2.34, wherein the subject does not have a history of significant autoimmune disease.
2.36 Any of Methods 2-2.35, wherein the subject has not received organ or bone marrow transplants.
2.37 Any of Methods 2-2.36, wherein the subject has IFP of 10-50 mmHg, 15-45 mmHg or 20-40 mmHg.
2.38 Any of Methods 2-2.37, wherein the subject has IFP of at least 15 mmHg, at least 20 mmHg, at least 25 mmHg, at least 30 mmHg, at least 35 mmHg, at least 40 mmHg, at least 45 mmHg or at least 50 mmHg.
2.39 Any of Methods 2-2.38, wherein the IFP is measured by a micropuncture technique, a wick-in-needle technique or MRI technology.

In another embodiment, the invention provides use of a compound, or a pharmaceutically acceptable salt or prodrug thereof, as defined in any one of Methods 1-1.10, in the manufacture of a medicament for treating a cancer in a subject with a tumor having interstitial fluid pressure (IFP) of at least 10 mmHg, together with an antibody binding to PD-1/PD-L1, e.g., for any one of Methods 2-2.39.

In another embodiment, the invention provides a compound which is an inhibitor of the interaction between the PD-1 receptor and its ligand PD-L1 and which is not a protein, e.g., a compound as defined in any one of Methods 1.1-1.10, for use in combination with an antibody binding to PD-1/PD-L1, in treating a cancer in a subject with a tumor having interstitial fluid pressure (IFP) of at least 10 mmHg, e.g., for use in any one of Methods 2-2.39.

In another aspect, the invention provides a method (Method 3) for treating a cancer in a subject having a tumor with interstitial fluid pressure (IFP) at least 10 mmHg, comprising administering to the subject a therapeutically effective amount of (i) a compound or a pharmaceutically acceptable salt or prodrug thereof, wherein the compound is non-protein inhibitor of the interaction between the PD-1 receptor and its ligand PD-L1, and (ii) an inhibitor of the CTLA-4/B7 interaction:
3.1 Method 3, wherein the compound binds PD-L1.
3.2 Any of Method 3 or 3.1, wherein the compound has a molecular weight (MW) less than 1500 Daltons.
3.3 Any of Methods 3-3.2, wherein the compound has an IC₅₀ of less than 100 nM a PD-1/PD-L1 binding assay.
3.4 Any of Methods 3-3.3, wherein the compound has the structure as described in any of Methods 1.2-1.10, in free or pharmaceutically acceptable salt form.
3.5 Any of Methods 3-3.4, wherein the compound has the structure of

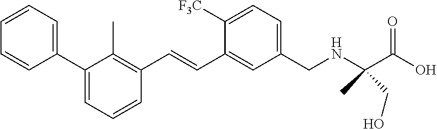 [SMILES: Cc1c(/C=C/c2cc(CN[C@@](C)(CO)C(=O)O)ccc2C(F)(F)F)cccc1-c1ccccc1]

in free or pharmaceutically acceptable salt form.
3.6 Any of Methods 3-3.5, wherein the cancer is bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or combinations of the cancers.
3.7 Any of Methods 3-3.6, wherein the cancer is breast cancer.
3.8 Any of Methods 3-3.6, wherein the cancer is melanomas.
3.9 Any of Methods 3-3.6, wherein the cancer is colorectal cancer.
3.10 Any of Methods 3-3.9, wherein the subject is a human.
3.11 Any of Methods 3-3.10, wherein the compound is simultaneously administered with the antibody.
3.12 Any of Methods 3-3.11, wherein the compound is administered subsequent to the administration of the inhibitor of the CTLA-4/B7 interaction.
3.13 Any of Methods 3-3.12, wherein the inhibitor of the CTLA-4/B7 interaction is ipilimumab.
3.14 Any of Methods 3-3.13, wherein the compound is administered orally.
3.15 Any of Methods 3-3.14, wherein the inhibitor of the CTLA-4/B7 interaction is administered as an intravenous infusion or subcutaneously.
3.16 Any of Methods 3-3.15, wherein the subject has previously received cancer treatment.
3.17 Method 3.16, wherein the cancer treatment is chemotherapy.
3.18 Method 3.17, wherein the chemotherapy comprises a platinum containing chemotherapeutic agent.
3.19 Method 3.17 or 3.18, wherein the chemotherapy is platinum-containing doublet chemotherapy.
3.20 Any of Methods 3-3.19, wherein the subject is not responsive to the cancer treatment.
3.21 Any of Methods 3-3.20, wherein the subject does not have a history of significant autoimmune disease.
3.22 Any of Methods 3-3.21, wherein the subject has not received organ or bone marrow transplants.
3.23 Any of Methods 3-3.22, wherein the compound is administered at a total dose of 20-300 mg/kg, 30-240 mg/kg, 40-200 mg/kg, 50-190 mg/kg, 60-180 mg/kg, 70-170 mg/kg, 80-160 mg/kg, 90-150 mg/kg or 100-140 mg/kg per day.
3.24 Any of Methods 3-3.23, wherein the compound is administered at an amount of about 10-150 mg/kg, 15-120 mg/kg, 20-100 mg/kg, 30-90 mg/kg, or 40-80 mg/kg body weight twice a day (BID).
3.25 Any of Methods 3-3.24, wherein the compound is administered at an amount of about 30 mg/kg, 35 mg/kg, 40 mg/kg, 45 mg/kg, 50 mg/kg, 55 mg/kg, 60 mg/kg, 65 mg/kg, 70 mg/kg, 75, mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg or 150 mg/kg body weight BID.
3.26 Any of Methods 3-3.25, wherein the compound is administered at an amount of about 30 mg/kg body weight BID.
3.27 Any of Methods 3-3.25, wherein the compound is administered at an amount of about 60 mg/kg body weight BID.
3.28 Any of Methods 3-3.25, wherein the compound is administered at an amount of about 90 mg/kg body weight BID.
3.29 Any of Methods 3-3.25, wherein the compound is administered at an amount of about 120 mg/kg body weight BID.
3.30 Any of Methods 3-3.29, wherein the subject has previously received cancer treatment.
3.31 Method 3.30, wherein the cancer treatment is chemotherapy.
3.32 Method 3.31, wherein the chemotherapy comprises a platinum containing chemotherapeutic agent.
3.33 Method 3.31 or Method 3.32, wherein the chemotherapy is platinum-containing doublet chemotherapy.
3.34 Any of Methods 3-3.33, wherein the subject is not responsive to the cancer treatment.
3.35 Any of Methods 3-3.34, wherein the subject does not have a history of significant autoimmune disease.
3.36 Any of Methods 3-3.35, wherein the subject has not received organ or bone marrow transplants.
3.37 Any of Methods 3-3.36, wherein the subject has IFP of 10-50 mmHg, 15-45 mmHg or 20-40 mmHg.
3.38 Any of Methods 3-3.37, wherein the subject has IFP of at least 15 mmHg, at least 20 mmHg, at least 25 mmHg, at least 30 mmHg, at least 35 mmHg, at least 40 mmHg, at least 45 mmHg or at least 50 mmHg.
3.39 Any of Methods 3-3.38, wherein the IFP is measured by a micropuncture technique, a wick-in-needle technique or MRI technology.

In another embodiment, the invention provides use of a compound, or a pharmaceutically acceptable salt or prodrug thereof, as defined in any one of Methods 1-1.10, in the manufacture of a medicament for treating a cancer in a subject with a tumor having interstitial fluid pressure (IFP) of at least 10 mmHg, together with an inhibitor of the CTLA-4A/B7 interaction, e.g., for any one of Methods 3-3.39.

In another embodiment, the invention provides a compound which is an inhibitor of the interaction between the PD-1 receptor and its ligand PD-1-1 and which is not a protein, e.g., a compound as defined in any one of Methods 1.1-1.10, for use in combination with an inhibitor of the CTLA-4A/B7 interaction, in treating a cancer in a subject with a tumor having interstitial fluid pressure (IFP) of at least 10 mmHg, e.g., for use in any one of Methods 3-3.39.

In another aspect, the invention provides a method (Method 4) for treating a cancer in a subject having a tumor with interstitial fluid pressure (IFP) at least 10 mmHg, comprising administering to the subject a therapeutically effective amount of a compound or a pharmaceutically acceptable salt or prodrug thereof and an inhibitor binding to vascular endothelial growth factor (VEGF):
4.1 Method 4, wherein the compound is an inhibitor of the interaction between the PD-1 receptor and its ligand PD-L1.
4.2 Any of Method 4 or 4.1, wherein the compound has a molecular weight (MW) of less than 1500 Daltons.
4.3 Any of Methods 4-4.2, wherein the compound has an IC₅₀ of less than 100 nM in a PD-1/PD-L1 binding assay;
4.4 Any of Methods 4-4.3, wherein the compound has the structure as described in any of Methods 1.2 - 1.10, in free or pharmaceutically acceptable salt form, e.g., the structure

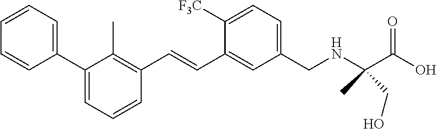 [SMILES: Cc1c(/C=C/c2cc(CN[C@@](C)(CO)C(=O)O)ccc2C(F)(F)F)cccc1-c1ccccc1]

in free or pharmaceutically acceptable salt form.
4.5 Any of Methods 4-4.4, wherein the cancer is bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, or combinations of the cancers.
4.6 Any of Methods 4-4.5, wherein the cancer is breast cancer.
4.7 Any of Methods 4-4.5, wherein the cancer is melanomas.
4.8 Any of Methods 4-4.5, wherein the cancer is colorectal cancer.
4.9 Any of Methods 4-4.8, wherein the subject is a human.
4.10 Any of Methods 4-4.9, wherein the compound is administered orally.
4.11 Any of Methods 4-4.9, wherein the compound is administered as an intravenous infusion, subcutaneously or by injection.
4.12 Any of Methods 4-4.11, wherein the inhibitor binding to VEGF is administered as an intravenous infusion, subcutaneously or orally.
4.13 Any of Methods 4-4.12, wherein the compound is simultaneously administered with the inhibitor binding to VEGF.
4.14 Any of Methods 4-4.12, wherein the compound is administered subsequent to the administration of the inhibitor binding to VEGF.
4.15 Any of Methods 4-4.14, wherein the subject has previously received cancer treatment.
4.16 Method 4.15, wherein the cancer treatment is chemotherapy.
4.17 Method 4.16, wherein the chemotherapy comprises a platinum containing chemotherapeutic agent.
4.18 Method 4.16 or Method 4.17, wherein the chemotherapy is platinum-containing doublet chemotherapy.
4.19 Any of Methods 4.15-4.18, wherein the subject is not responsive to the cancer treatment.
4.20 Any of Methods 4-4.19, wherein the subject does not have a history of significant autoimmune disease.
4.21 Any of Methods 4-4.20, wherein the subject has not received organ or bone marrow transplants.
4.22 Any of Methods 4-4.21, wherein the compound is administered at a total dose of 20-300 mg/kg, 30-240 mg/kg, 40-200 mg/kg, 50-190 mg/kg, 60-180 mg/kg, 70-170 mg/kg, 80-160 mg/kg, 90-150 mg/kg or 100-140 mg/kg per day.
4.23 Any of Methods 4-4.22, wherein the compound is administered at an amount of about 10-150 mg/kg, 15-120 mg/kg, 20-100 mg/kg, 30-90 mg/kg, or 40-80 mg/kg body weight twice a day (BID).
4.24 Any of Methods 4-4.23, wherein the compound is administered at an amount of about 30 mg/kg, 35 mg/kg, 40 mg/kg, 45 mg/kg, 50 mg/kg, 55 mg/kg, 60 mg/kg, 65 mg/kg, 70 mg/kg, 75, mg/kg, 80 mg/kg, 85 mg/kg, 90 mg/kg, 95 mg/kg, 100 mg/kg, 105 mg/kg, 110 mg/kg, 115 mg/kg, 120 mg/kg, 125 mg/kg, 130 mg/kg, 135 mg/kg, 140 mg/kg, 145 mg/kg or 150 mg/kg body weight BID.
4.25 Any of Methods 4-4.24, wherein the compound is administered at an amount of about 30 mg/kg body weight BM.
4.26 Any of Methods 4-4.24, wherein the compound is administered at an amount of about 60 mg/kg body weight BM.
4.27 Any of Methods 4-4.24, wherein the compound is administered at an amount of about 90 mg/kg body weight BM.
4.28 Any of Methods 4-4.24, wherein the compound is administered at an amount of about 120 mg/kg body weight BID.
4.29 Any of Methods 4-4.28, wherein the inhibitor binding to VEGF is selected from a group consisting of bevacizumab, pazopanib, cabozantinib, sorafenib, axitinib, regorafenib, ponatinib, cabozantinib, vandetanib, ramucirumab, lenvatinib, and bevacizumab.
4.30 Any of Methods 4-4.29, wherein the subject has IFP of 10-50 mmHg, 15-45 mmHg or 20-40 mmHg.
4.31 Any of Methods 4-4.30, wherein the subject has IFP of at least 15 mmHg, at least 20 mmHg, at least 25 mmHg, at least 30 mmHg, at least 35 mmHg, at least 40 mmHg, at least 45 mmHg or at least 50 mmHg.
4.32 Any of Methods 4-4.31, wherein the IFP is measured by a micropuncture technique, a wick-in-needle technique or MRI technology.

In another embodiment, the invention provides use of a compound, or a pharmaceutically acceptable salt or prodrug thereof, as defined in any one of Methods 1-1.10, in the manufacture of a medicament for treating a cancer in a subject with a tumor having interstitial fluid pressure (IFP) of at least 10 mmHg, together with an inhibitor binding to vascular endothelial growth factor (VEGF), e.g., for any one of Methods 4-4.32.

In another embodiment, the invention provides a compound which is an inhibitor of the interaction between the PD-1 receptor and its ligand PD-L1 and which is not a protein, e.g., a compound as defined in any one of Methods 1.1-1.10, for use in combination with an inhibitor binding to vascular endothelial growth factor (VEGF), in treating a cancer in a subject with a tumor having interstitial fluid pressure (IFP) of at least 10 mmHg, e.g., for use in any one of Methods 4-4.32.

For oral administration, a pharmaceutical composition comprising the compounds as disclosed herein may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) or Formula (II) in an oily phase, such as, for example, a mixture of soybean oil and lecithin; 2) combining the Formula (I) or Formula (II) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this disclosure include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this disclosure can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this disclosure depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 250 mg/kg body weight, preferably between about 0.0025 and about 150 mg/kg body weight and most preferably between about (0.005 to 120 mg/kg body weight, may be appropriate. The daily dose can be administered in one to four doses per day. Other dosing schedules include one dose per week and one dose per two-day cycle.

For therapeutic purposes, the active compounds of this disclosure are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a. dispersion of active compound in hydroxypropylmethyl cellulose.

PD-L1 blockade may also be combined with standard cancer treatments. PD-L1 blockade may be effectively combined with chemotherapeutic regimes. In these instances, it may be possible to reduce the dose of chemotherapeutic reagent administered (Mokyr, M. et al. (1998) Cancer Research 58: 5301-5304). PD-L1 blockade may also be combined with existing antibodies binding to PD-1 or PD-L1.

Tumors evade host immune surveillance by a large variety of mechanisms. Many of these mechanisms may be overcome by the inactivation of proteins which are expressed by the tumors and which are immunosuppressive. These include among others TGF-beta (Kehrl, J. et al. (1986) J. Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). Macrocyclic peptides to each of these entities may be used in combination with the compounds of this disclosure to counteract the effects of the immunosuppressive agent and favor tumor immune responses by the host.

Macrocyclic peptides that activate host immune responsiveness can be used in combination with anti-PD-1. These include molecules on the surface of dendritic cells which activate DC function and antigen presentation. Anti-CD40 macrocyclic peptides are able to substitute effectively for T cell helper activity (Ridge, J. et al. (1998) Nature 393: 474-478) and can be used in conjunction with PD-1 macrocyclic peptides (Ito, N. et al. (2000) Immunobiology 201 (5) 527-40). Activating macrocyclic peptides to T cell costimulatory molecules such as CTLA-4 (e.g., U.S. Pat. No. 5,811,097), OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al, (1999) Nature 397: 262-266) may also provide for increased levels of T cell activation.

Vascular endothelial growth factor (VEGF) is one of the most important proteins that promote angiogenesis, which is a tightly regulated process of developing new blood vessels from a pre-existing vascular network (Ferrara, N., (2004), Endocrine Reviews, 25(4): 581-611). Angiogenesis is required during development and normal physiological processes such as wound healing, and is also involved in a number of disease pathogenesis, including AMD. RA, Diabetic Retinopathy, tumor growth and metastasis. Inhibition of angiogenesis has been shown to be effective in therapeutic applications.

The compounds of the disclosure e.g., as described in Methods 1.2-1.10) can be co-administered with one or more other therapeutic agents, e.g., a cytotoxic agent, a radiotoxic agent or an immunosuppressive agent. The compounds of the disclosure can be administered before, after or concurrently with the other therapeutic agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, decarbazine and cyclophosphamide hydroxyurea which, by themselves, are only effective at levels which are toxic or subtoxic to a patient. Cisplatin is intravenously administered as a 100 mg/dose once every four weeks and adriamycin is intravenously administered as a 60-75 mg/ml dose once every 21 days. Co-administration of a compound of Formula (I), Formula (II) or salts thereof, with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibodies.

The above other therapeutic agents, when employed in combination with the compounds of the present disclosure, may be used, for example, in those amounts indicated in the Physicians' Desk Reference (PDR) or as otherwise determined by one of ordinary skill in the art. In the methods of the present disclosure, such other therapeutic agent(s) may be administered prior to, simultaneously with, or following the administration of the compounds as disclosed in Method 1 et seq., Method 2 et seq. Method 3 et seq. and Method 4, et seq.

### EXAMPLES

The disclosure is further defined in the following Examples. It should be understood that the Examples are given by way of illustration only. From the above discussion and the Examples, one skilled in the art can ascertain the essential characteristics of the disclosure, and without departing from the spirit and scope thereof, can make various changes and modifications to adapt the disclosure to various uses and conditions. As a result, the disclosure is not limited by the illustrative examples set forth hereinbelow, but rather is defined by the claims appended hereto.

### Example 1: Measurements of (IFP) in Various Tumors

Interstitial fluid pressure (IFP) was measured by a Millar Mikro-Tip pressure catheter transducer (SPR-1000). The catheter was connected to PCU-2000 Pressure Control Unit and an AD Instruments PowerLab data acquisition system (Millar Instruments, Inc.). After recording, data were analyzed using LabChart software (Millar Instruments, Inc.). The system was calibrated to 0 mmHg in a water column before each measurement. To place the catheter, an 18-gauge needle was first inserted into the center of each tumor, and the probe then was introduced into the space after needle withdrawal and held there until a stable pressure output signal was measured. The results are shown in Table 1 and indicate that IFP in these tumors are at least 10 mmHg.

TABLE 1
A summary of IFPs in various tumors

| | MC-38 | B16F10 | 4T1 |
|---|---|---|---|
| IFP | 15.54 | 16.08 | 48.3 |
| (mmHg) | 25.32 | 23.28 | 45.78 |
| | 45 | 12.72 | 74.64 |
| | / | / | 66 |
| | / | / | 54.42 |

### Example 2: In Vitro Studies of the Compound

Biological Assay: The ability of the compounds disclosed herein to bind to PD-L1 was investigated using a PD-1/PD-L1 Homogenous Time-Resolved Fluorescence (HTRF) binding assay.

All binding studies were performed in an HTRF assay buffer consisting of dPBS supplemented with 0.1% (with v) bovine serum albumin and 0.05% (v/v) Teen-20. For the PD-1-Ig/PD-L1-His binding assay, inhibitors were pre-incubated with PD-L1-His (10 nM final) for 15 m in 4 .mu.l of assay buffer, followed by addition of PD-1-Ig (20 nM final) in 1 .mu.l of assay buffer and further incubation for 15 m. PD-L 1 from either human, cyno, or mouse were used. HTRF detection was achieved using europium crypate-labeled anti-Ig (1 nM final) and allophycocyanin (APC) labeled anti-His (20 nM final). Antibodies were diluted in HTRF detection buffer and 5 .mu.l was dispensed on top of binding reaction. The reaction mixture was allowed to equilibrate for 30 minutes and signal (665 nm/620 nm ratio) was obtained using an EnVision fluorometer. Additional binding assays were established between PD-i-Ig/PD-L2-His (20 & 5 nM, respectively), CD80-His/PD-L1-Ig (100 & 10 nM, respectively) and CD80-His/CTLA4-Ig (10 & 5 nM, respectively). Competition studies between biotinylated polypeptide (AISGGGGSTYYADSVKD) and human PD-L1-His were performed as follows. Inhibitors were pre-incubated with PD-L1-His (10 nM final) for 60 m in 4 .mu.l of assay buffer followed by addition of biotinylated polypeptide (0.5 nM final) in 1 .mu.l of assay buffer. Binding was allowed to equilibrate for 30 m followed by addition of europium crypated labeled Strepatavidin (2.5 pM final) and APC-labeled anti-His (20 nM final) in 5 .mu.l of HTRF buffer. The reaction was allowed to equilibrate for 30 m and signal (665 nm/620 nm ratio) was obtained using an EnVision fluorometer. In the HTRF assay, the compound potently inhibited the binding between hPD-1 and hPD-L1 with IC₅₀ of 19 nM.

To measure the cellular activity of the compound, a protocol of Activation of T-Cell Suppressed by PD-L1 was used. In this protocol, human Hep3B cells were stably transfected with human PD-L1. The human T cells containing PD-1 were inactivated by co-culturing with these PD-L1 transfected cells. Then anti-PD-1 antibody Keytruda® was selected as the reference to profile the compound for its activation of PD-L1 suppressed human T-cells. In a dose dependently manner, the compound effectively restored the activation of the PD-L1 suppressed human T cell indicated by the increase of cytokine IFN-g as Keytruda was used as a positive control.

### Example 3 In Vivo Test of Anti-tumor Efficacy of the Compound in the Subcutaneous 4T1 Murine Breast Cancer Model in BALB/c Mice

Materials required for the experiment: Antibody: mouse PD-1 antibody, Product specifications: 7.09 mg/mL (50 mg/mL), Lot No.: 695318A1 purchased from BioXcell, storage at 4oC. Experiment animal: 60 BALB/C mice, female, 6˜8 weeks old, 20˜23 g, purchased from Shanghai Lingchang Biotechnology Co, Ltd. Formulation material: castor oil (Cremophor RH40), CAS No.: 61788-85-0, Lot No.: 29761847G0, purchased from Shanghai Xietai Chemical Co. Ltd.; β-cyclodextrin (SBE-β-CD), CAS No.:128446-35-5, Lot No.: 20180110, purchased from Shanghai Shaoyuan Chemical Co. Ltd.; RPMI-1640 culture medium, Art. No.: 1869036, Lot No.:11875-093, purchased from Gibco Co. Ltd.; PBS, Art. No.: SH30256.01, Lot No.: AB10141338, purchased from HyClone Co. Ltd.; Fetal bovine serum: CAS No.: 10099-141, Lot No.: 1966174C, purchased from Gibco Co. Ltd.

Cell preparation and implantation: 4T1 cells (CRL-2539™) were cultured with RPMI 1640 supplemented with 10% heat inactivated FBS at 37° C. in 5% CO₂ incubator. Cells were passaged 3 times a week. Cells were harvested, counted and passaged, inoculated when around 70% confluent.

Tumor cell inoculation and group administration: The 50 uL cell suspension containing 1×10⁵ 4T1 tumor cells (cells suspended in base RPMI-1640 medium) was inoculated into the fourth fat pad of the left abdomen of mice. On the second day after inoculation, according to the order of tumor inoculation, stratified randomization was used to group and start the administration on the day of grouping.

Preparation of test substances: Preparation of formulation: 490 mL of sterile water was added into the volumetric flask with magnetic stirring to have a vortex. 100 g of castor oil (Cremophor RH40) was added with a spoon slowly into the vortex and the solution was kept stirring. 200 g of β-cyclodextrin (SBE-β-CD) was added while the solution was kept stirring until the solution was clear, and the total volume was set to 1000 mL, which contained 10% (w/v) Cremophor RH40+20% (w/v) an aqueous solution of SBE-β-CD.

Preparation of Compound Suspension: 178.88 mg compound was weighed and 14.817 mL 10% (w/v) Cremophor RH40+20% (w/v) SBE-β-CD aqueous solution were added. The suspension solution with a concentration of 12.0 mg/mL was obtained by fully mixing with magnetic stirring. 7.0 mL of the compound suspension solution with concentration of 12.0 mg/mL was pipetted and 7.0 mL aqueous formulation solution was added. The suspension solution with a concentration of 6.0 mg/mL was obtained by fully mixing with magnetic stirring, 7.0 mL of the compound suspension solution with concentration of 6.0 mg/mL was pipetted and 7.0 mL aqueous formulation solution was added. The suspension solution with a concentration of 3.0 mg/mL was obtained by fully mixing with magnetic stirring. 7.0 mL of the compound suspension solution with concentration of 3.0 mg/mL was pipetted and 7.0 mL aqueous formulation solution was added. The suspension solution with a concentration of 1.5 mg/mL was obtained by fully mixing with magnetic stirring. The compound suspension solution was prepared once a day.

Preparation of mPD-1 Antibody: 0.339 mL mPD-L1 antibody (7.09 mg/mL) original solution was pipetted and 2.061 mL PBS solution was added. The solution was fully mixed and the final concentration of 1 mg/mL solution was obtained.

Procedure: The mice in the vehicle group were weighed and recorded in the electronic balance according to their numbers. The mice in the vehicle group were given prepared formulation solution twice a day by oral administration according to their body weight with a capacity of 0.1 mL/10 g. Each mouse spent about 20 seconds and 10 mice in each group shared for about 5 minutes.

The mice in the antibody (10 mg/kg) group were weighed and recorded in the electronic balance according to their numbers. The mice in the antibody group were given prepared antibody solution twice a week by IP administration according to their body weight with a capacity of 0.1 ml/10 g. Each mouse spent about 20 seconds and 10 mice in each group shared for about 5 minutes.

The mice in the compound (15 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the compound (30 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the compound (60 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the compound (120 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

Tumors were measured with digital vernier calipers three times a week and calculating the volume of tumors. Euthanasia was imposed if the size of the tumor exceeds 2000 mm³, or when the animal has serious disease, pain, or is unable to freely eat and drink water. The body weight of the animals was measured by electronic balance every day. Euthanasia is required when the animal is obviously thin and its weight is reduced by more than 20%. The experiment ended 20 days after compound was administered.

The tumor inhibition rate was calculated as:

TGI (%)=(1−(the volume of the tumor on the day of Administration−the volume of the tumor on the first day of administration)/(the volume of the tumor on the day of Administration−the volume of the tumor on the first day of vehicle group)×100%.

With GraphPad Prism 5.0 software, the tumor volume changes in mice were analyzed by Two-way ANOVA and compared with the vehicle group according to the Bonferroni posttests method, P<0.05 was considered to be significantly different.

In the assay, the compound and mPD-1 antibody demonstrated similar efficacy in tumor growth inhibition (TGI). Furthermore, the minimum effective dose of the compound was 30 mpk (p<0.05). The results are summarized in Table 2.

TABLE 2
Results of In vivo test of anti-tumor efficacy of the compound in
the subcutaneous 4T1 murine breast cancer model in BALB/c mice.

| Groups | Tumor Volume (mm³) | TGI (%) | p-Value |
|---|---|---|---|
| Blank Control | 860.89 ± 42.52 | — | — |
| mPD-1 antibody, | 700.96 ± 39.56 | 18.58 | <0.001 |
| 10 mg/kg, IP, BIW | | | |
| Compound, 15 mg/kg, | 689.03 ± 43.97 | 19.96 | <0.001 |
| PO, BID | | | |
| Compound, 30 mg/kg, | 573.9 ± 43.18 | 33.34 | <0.001 |
| PO, BID | | | |
| Compound, 60 mg/kg, | 548.24 ± 31.39 | 36.32 | <0.001 |
| PO, BID | | | |
| Compound, 120 mg/kg, | 503.16 ± 32.93 | 41.55 | <0.001 |
| PO, BID | | | |

### Example 4 In Vivo Test of Anti-tumor Efficacy of the Compound in the B16F10 Models

Materials required for the experiment: Antibody: mouse PD-1 antibody, Product specifications: 7.09 mg/mL (50 mg/mL), Lot No.: 695318A1 purchased from BioXcell, storage at 4oC. Experimental animals: 60 C57BL/6 mice, female, 6˜8 weeks old, 17˜21 g, purchased from Shanghai Lingchang Biotechnology Co. Ltd. Formulation materials: castor oil (Cremophor RH40) , CAS No.: 61788-85-0, Lot No.: 29761847G0, purchased from Shanghai Xietai Chemical Co. Ltd.; β-cyclodextrin (SBE-β-CD), CAS No.: 128446-35-5, Lot No.: 20180110, purchased from Shanghai Shaoyuan Chemical Co. Ltd.; DMEM culture medium, Art. No.: 11995-065, Lot No.:2025378, purchased from Gibco Co. Ltd.; PBS, Art. No.: SH30256.01, Lot No.: AB10141338, purchased from HyClone Co. Ltd.; Fetal bovine serum: Art. No.: 04-002-1A, Lot No.: 1625436, purchased from Boehringer Ingelheim Co. Ltd.; Methyl cellulose (MC), Art. No.: M7027-250G, Lot No.: 079K0054V, purchased from Sigma.

Cell preparation and implantation: The B16-F10 tumor cells (ATCC CRL6475™) were maintained in vitro as a monolayer culture in DMEM medium supplemented with 10% heat inactivated fetal bovine serum at 37° C. in an atmosphere of 5% CO₂ in air. The tumor cells were routinely subcultured three times weekly by trypsin-EDTA treatment. The cells growing to a confluency around 70%-80% were harvested and counted for tumor inoculation.

Tumor cell inoculation and group administration: The 100 uL cell suspension containing 1×10⁶ B16F10 tumor cells (cells suspended in base DMEM medium) was inoculated into the right subcutaneous of mice. On the second day after inoculation, according to the order of tumor inoculation, stratified randomization was used to group and start the administration on the day of grouping.

Preparation of test substances: Preparation of formulation: 700 mL of sterile water was added into the volumetric flask with magnetic stirring to have a vortex. 100 g of castor oil (Cremophor RH40) was added with a spoon slowly into the vortex and the solution was kept stirring. 200 g of β-cyclodextrin (SBE-β-CD) was added while the solution was kept stirring until the solution was clear, and the total volume was set to 1000 mL, which contained 10% (w/v) Cremophor RH40+20% (w/v) an aqueous solution of SBE-β-CD.

Preparation of compound suspension: 169.16 mg compound was weighed, 14.012 mL 10% (w/v) Cremophor RH40+20% (w/v) SBE-β-CD aqueous solution were added, and the suspension solution with a concentration of 12.0 mg/mL was obtained by fully mixing with magnetic stirring. 6.0 mL of the compound suspension solution with concentration of 12.0 mg/mL was pipetted and 6.0 mL aqueous formulation solution was added. The suspension solution with a concentration of 6.0 mg/mL was obtained by fully mixing with magnetic stirring. 6.0 mL of the compound suspension solution with concentration of 6.0 mg/mL was pipetted and 6.0 mL aqueous formulation solution was added. The suspension solution with a concentration of 3.0 mg/mL was obtained by fully mixing with magnetic stirring. The compound suspension solution was prepared once a day.

Preparation of mPD-1 antibody: 0.564 mL mPD-L1 antibody (7.09 mg/mL) original solution was pipetted and 3.307 mL PBS solution was added. The solution was fully mixed and the final concentration of 1 mg/mL solution was obtained.

Procedure: The mice in the vehicle group were weighed and recorded in the electronic balance according to their numbers. The mice in the vehicle group were given prepared formulation solution twice a day by oral administration according to their body weight with a capacity of 0.1 mL/10 g. Each mouse spent about 20 seconds and 10 mice in each group shared for about 5 minutes.

The mice in the antibody (10 mg/kg) group were weighed and recorded in the electronic balance according to their numbers. The mice in the antibody group were given prepared antibody solution twice a week by IP administration according to their body weight with a capacity of 0.1 ml/10 g. Each mouse spent about 20 seconds and 10 mice in each group shared for about 5 minutes.

The mice in the compound (30 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the compound (60 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the compound (120 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the combo group (compound, 60 mg/kg; mPD-1, 10 mg/kg) were weighed and recorded in the electronic balance according to their numbers. The mice in this group were given prepared compound suspension twice a day by oral administration according to their body weight with a capacity of 0.1 ml/10 g and mouse antibody solution twice a week by IP administration according to their body weight with a capacity of 0.1 ml/10 g. Each mouse spent about 20 seconds and 10 mice in each group shared for about 5 minutes.

Tumors were measured with digital vernier calipers three times a week and calculating the volume of tumors. Euthanasia was imposed if the size of the tumor exceeds 2000 mm³, or the animal has serious disease, pain, or is unable to freely eat and drink water. The body weight of the animals was measured by electronic balance every day. Euthanasia is required when the animal is obviously thin and its weight is reduced by more than 20%. The experiment ended 20 days after compound was administered.

The tumor inhibition rate was calculated as:

TGI (%)=(1−(the volume of the tumor on the day of administration-the volume of the tumor on the first day of administration)/(the volume of the tumor on the day of administration−the volume of the tumor on the first day of vehicle group)×100%.

Using GraphPa.d Prism 5.0 software, the tumor volume changes in mice were analyzed by Two-way ANOVA and compared with the vehicle group according to the Bonferroni posttests method, P<0.05 was considered to be significantly different.

The results showed that compound could significantly inhibit the growth of subcutaneous transplanted melanoma cell line in mice and it was well tolerated in C57BL/6 mice without obvious adverse reactions. The results are summarized in Table.

TABLE 3
Results of in vivo tests of anti-tumor efficacy of
the compound in the B16F10 models

| Groups | Tumor Volume (mm³) | TGI (%) | p-Value |
|---|---|---|---|
| Blank Control | 1235.87 + 220.28 | — | — |
| mPD-1 antibody, | 693.74 ± 272.23 | 43.87 | 0.0007 |
| 10 mg/kg, IP, BIW | | | |
| Compound, 30 mg/kg, | 795.99 ± 112.92 | 35.59 | 0.0098 |
| PO, BID | | | |
| Compound, 60 mg/kg, | 755.48 ± 155.85 | 38.87 | 0.0037 |
| PO, BID | | | |
| Compound, 120 mg/kg, | 650.67 ± 157.53 | 47.35 | 0.0002 |
| PO, BID | | | |
| mPD-1antibody, | 487.05 ± 131.39 | 60.59 | <0.0001 |
| 10 mg/kg, IP, BIW + | | | |
| Compound, 60 mg/kg, | | | |
| PO, BID | | | |

### Example 5 In Vivo Test of Anti-tumor Efficacy of the Compound and PD-L1 Antibody Durvalumab in MC-38-hPD-L1 Models

Materials required for the experiment: Antibody: PD-L1 antibody Durvalumab, Product specifications: 120 mg/2.4 mL(50 mg/mL), Lot No.: 041E17C, Manufacture: AstraZeneca, purchased from Hongkong Mingchuang Medical Limited, storage at 2-8° C. Experimental animal: 120 C57BL/6-hPD-1 mice, female, 6˜8 weeks old, 18˜21 g, purchased from Jiangsu Gem Pharmatech Co. Ltd. Formulation material: castor oil (Cremophor RH40), CAS No.: 61788-85-0, Lot No.: 29761847G0, purchased from Shanghai Xietai Chemical Co. Ltd.; β-cyclodextrin (SBE-β-CD), CAS No.: 128446-35-5, Lot No.: R1804474, purchased from Shanghai Shaoyuan Chemical Co. Ltd.; DMEM culture medium, CAS No.: 11995-065, Lot No.: 2025378, purchased from Gibco Co. Ltd.; PBS, Art. No.: SH30256.01, Lot No.: AB10141338, purchased from HyClone Co. Ltd.; Fetal bovine serum: CAS No.: 10099-141, Lot No.: 1966174C, purchased from Gibco Co. Ltd.; hygromycinB: CAS No.: 10687010, Lot No.: HY069-L12 purchased from Invitrogen.

Cell culture: The MC-38 tumor cells (NCI) were maintained in vitro as a monolayer culture in DMEM medium supplemented with 10% heat inactivated fetal bovine serum, 100 μg/mL hygromycinB at 37° C. in an atmosphere of 5% CO₂ in air. The tumor cells were routinely subcultured three times weekly by trypsin-EDTA treatment. The cells growing to a confluency around 70%-80% were harvested and counted for tumor inoculation.

Tumor cell inoculation and group administration: The 100 uL cell suspension containing 1×10⁶ MC-38-hPD-L1 tumor cells (cells suspended in base DMEM medium) was inoculated into right dorsal subcutaneous area in mice. 6 days after inoculation, 60 tumor-bearing mice with transplanted tumors ranging from 31.49 mm³ to 110.26 mm³ were selected, stratified randomization was used to group and start the administration on the day of grouping.

Preparation of test substances: Preparation of formulation: 800 mL of sterile water was added into the volumetric flask with magnetic stirring to have a vortex. 100 g of castor oil (Cremophor RH40) was added with a spoon slowly into the vortex and the solution was kept stirring. 200 g of β-cyclodextrin (SBE-β-CD) was added while the solution was kept stirring until the solution was clear, and the total volume was set to 1000 mL, which contained 10% (w/v) Cremophor RH40+20% (w/v) an aqueous solution of SBE-β-CD.

Preparation of Compound Suspension: 150.92 mg compound was weighed and 12.5 mL 10% (w/v) Cremophor RH40+20% (w/v) SBE-β-CD aqueous solution were added. The suspension solution with a concentration of 12.0 mg/mL was obtained by vortex for 2 minutes and ultrasound for 30 minutes. 5.0 mL of the compound suspension solution with concentration of 12.0 mg/mL was pipetted and 5.0 mL aqueous formulation solution was added. The suspension solution with a concentration of 6.0 mg/mL was obtained by vortex for 1 minute and sonicated by ultrasound for 5 minutes. 2.0 mL of the compound suspension solution with concentration of 6.0 mg/mL was pipetted and 6.0 mL aqueous formulation solution was added. The suspension solution with a concentration of 3.0 mg/mL was obtained by vortex for 1 minute and sonicated by ultrasound for 5 minutes. Compound suspension solution was prepared once a day.

Preparation of hPD-L1 antibody: 0.12 mL PD-L1 antibody Durvalumab (50 mg/mL) original solution was pipetted and divided into 6 portions in 5 mL sterile centrifugal tube, which were packed with each containing 6.0 mg. They were stored in refrigerator at 4° C. Before administration, a raw solution containing 0.12 mL and 50 mg/mL was taken, and 2.88 mL 0.9% sodium chloride solution was added to make the solution thoroughly mixed. 3 mL Duvalumab solution with the concentration 2 mg/mL was obtained.

Procedure: The mice in the vehicle group were weighed and recorded in the electronic balance according to their numbers. The mice in the vehicle group were given prepared formulation solution twice a day by oral administration according to their body weight with a capacity of 0.1 mL/10 g. Each mouse spent about 20 seconds and 10 mice in each group shared for about 5 minutes.

The mice in the compound (30 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the compound (60 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the compound (120 mg/kg) group were weighed and recorded in the electronic balance according to their numbers. The mice in this group were given prepared compound suspension twice a day by oral administration according to their body weight with a capacity of 0.1 ml/10 g. Each mouse spent about 20 seconds and 10 mice in each group shared for about 5 minutes.

The mice in the combo group (compound. 60 mg/kg; Duvalutnab, 20 mg/kg) were weighed and recorded in the electronic balance according to their numbers. The mice in this group were given prepared compound suspension twice a day by oral administration according to their body weight with a capacity of 0.1 ml/10 g and Duvalumab twice a week by IP administration according to their body weight with a capacity of 0.1 ml/10 g. Each mouse spent about 20 seconds and 10 mice in each group shared for about 5 minutes.

The mice in Duvalumab (20 mg/kg) group were weighed and recorded in the electronic balance according to their numbers. The mice in the antibody group were given prepared antibody solution twice a week by IP administration according to their body weight with a capacity of 0.1 ml/10 g. Each mouse spent about 20 seconds and 10 mice in each group shared for about 5 minutes.

Tumors were measured with digital vernier calipers three times a week and calculating the volume of tumors. Euthanasia was imposed if the size of the tumor exceeds 2000 mm³, or if the animal has serious disease, pain, or is unable to freely eat and drink water. The body weight of the animals is measured by electronic balance every day. Euthanasia is required when the animal is obviously thin and its weight is reduced by more than 20%. The experiment ended 19 days after compound was administered.

The tumor inhibition rate was calculated as:

TGI(%)=(1−(the volume of the tumor on the day of Administration−the volume of the tumor on the first day of administration)/(the volume of the tumor on the day of Administration−the volume of the tumor on the first day of vehicle group)×100%.

With GraphPad Prism 5.0 software, the tumor volume changes in mice were analyzed by Two-way ANOVA and compared with the vehicle group according to the Bonferroni posttests method, P<0.05 was considered to be significantly different.

The results showed that compound could significantly inhibit the growth of subcutaneous transplanted melanoma cell line in mice and it was well tolerated in C57BL/6-hPD-1 mice without obvious adverse reactions. The results are summarized in Table 4.

TABLE 4
Results of in vivo test of anti-tumor efficacy of the compound
and PD-L1 antibody Durvalumab in MC-38-hPD-L1 models

| Groups | Tumor Volume (mm³) | TGI (%) | p-Value |
|---|---|---|---|
| Blank Control | 588.68 ± 58.56 | — | — |
| Durvalumab | 341.19 ± 56.59 | 48.17 | <0.0001 |
| (anti-hPD-L1), | | | |
| 20 mg/kg, IP, BIW | | | |
| Compound, 30 mg/kg, | 459.82 ± 58.24 | 24.95 | 0.0309 |
| PO, BID | | | |
| Compound, 60 mg/kg, | 439.04 ± 51.23 | 29.06 | 0.0075 |
| PO, BID | | | |
| Compound, 120 mg/kg, | 325.23 ± 38.59 | 51.21 | <0.0001 |
| PO, BID | | | |
| Compound, 60 mg/kg, | 265.55 ± 64.11 | 62.82 | <0.0001 |
| PO, BID + Durvalumab | | | |
| (anti-hPD-L1), | | | |
| 20 mg/kg, IP, BIW | | | |

While the present invention has been described with reference to embodiments, it will be understood by those skilled in the art that various modifications and variations may be made therein without departing from the scope of the present invention as defined by the appended claims.

1. A method for treating a cancer in a subject having a tumor with interstitial fluid pressure (IFP) of at least 10 mmHg, comprising administering to the subject a therapeutically effective amount of a compound or a pharmaceutically acceptable salt or prodrug thereof, wherein the compound is an inhibitor of the interaction between the PD-1 receptor and its ligand PD-L1 and wherein the compound is not a protein.

2. The method of claim 1, wherein the compound is an inhibitor of PD-L1.

3. The method of claim 1, wherein the compound has a molecular weight (MW) of less than 1500 Daltons.

4. The method of claim 1, wherein the compound has an IC₅₀ of less than 100 nM in a PD-1/PD-L1 binding assay.

5. The method of claim 1, wherein the compound binds to PD-L1.

6. The method of claim 1, wherein the compound is selected from a group consisting of: 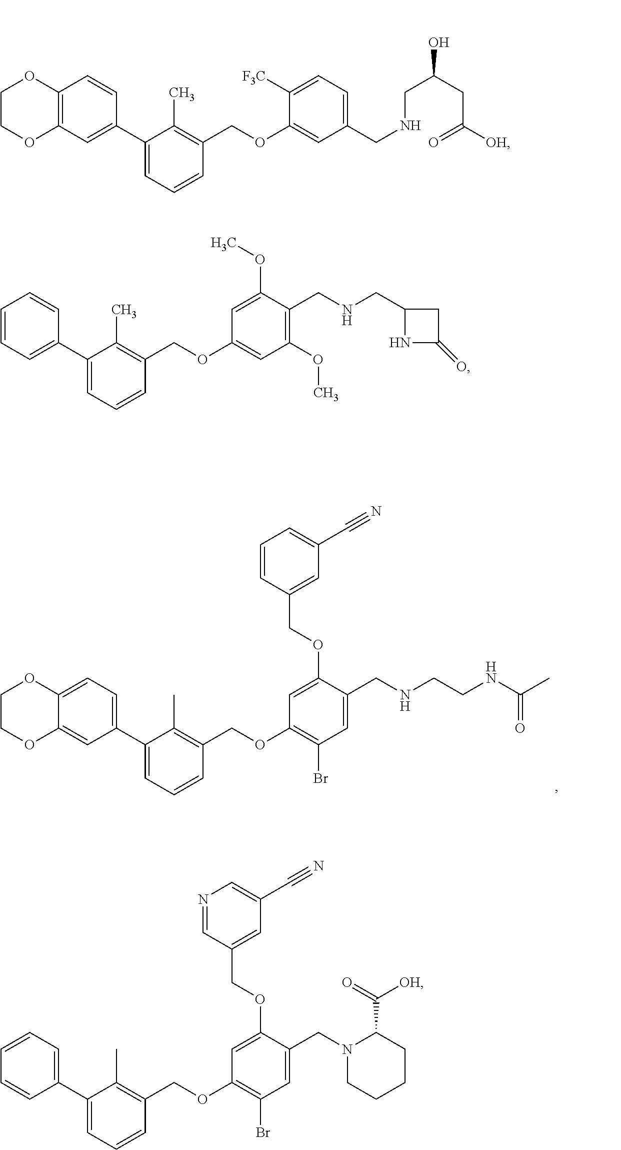 [SMILES: Cc1c(COc2cc(CNC[C@@H](O)CC(=O)O)ccc2C(F)(F)F)cccc1-c1ccc2c(c1)OCCO2 ; COc1cc(OCc2cccc(-c3ccccc3)c2C)cc(OC)c1CNCC1CC(=O)N1 ; CC(=O)NCCNCc1cc(Br)c(OCc2cccc(-c3ccc4c(c3)OCCO4)c2C)cc1OCc1cccc(C#N)c1 ; Cc1c(COc2cc(OCc3cncc(C#N)c3)c(CN3CCCC[C@H]3C(=O)O)cc2Br)cccc1-c1ccccc1] 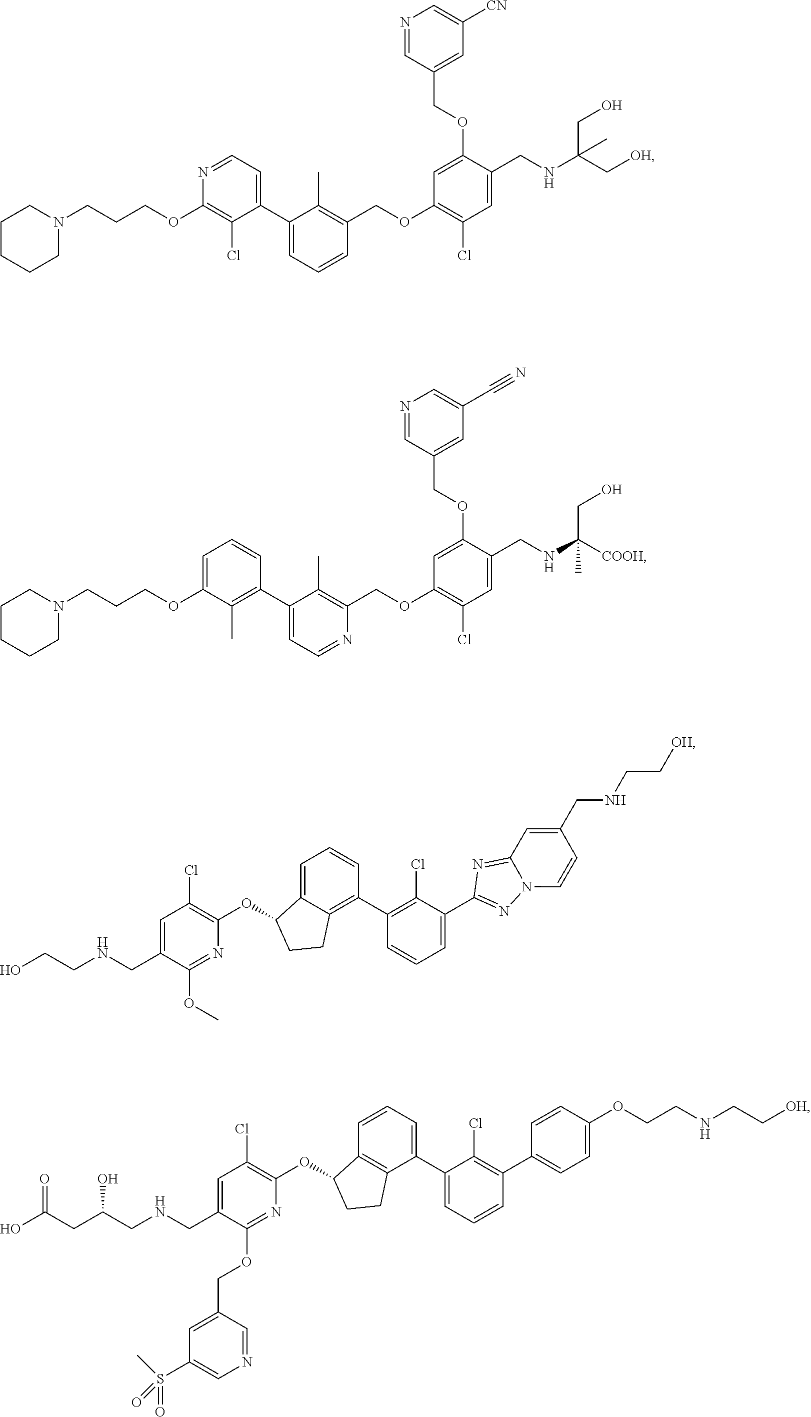 [SMILES: Cc1c(COc2cc(OCc3cncc(C#N)c3)c(CNC(C)(CO)CO)cc2Cl)cccc1-c1ccnc(OCCCN2CCCCC2)c1Cl ; Cc1c(OCCCN2CCCCC2)cccc1-c1ccnc(COc2cc(OCc3cncc(C#N)c3)c(CN[C@@](C)(CO)C(=O)O)cc2Cl)c1C ; COc1nc(O[C@H]2CCc3c(-c4cccc(-c5nc6cc(CNCCO)ccn6n5)c4Cl)cccc32)c(Cl)cc1CNCCO ; CS(=O)(=O)c1cncc(COc2nc(O[C@H]3CCc4c(-c5cccc(-c6ccc(OCCNCCO)cc6)c5Cl)cccc43)c(Cl)cc2CNC[C@@H](O)CC(=O)O)c1] 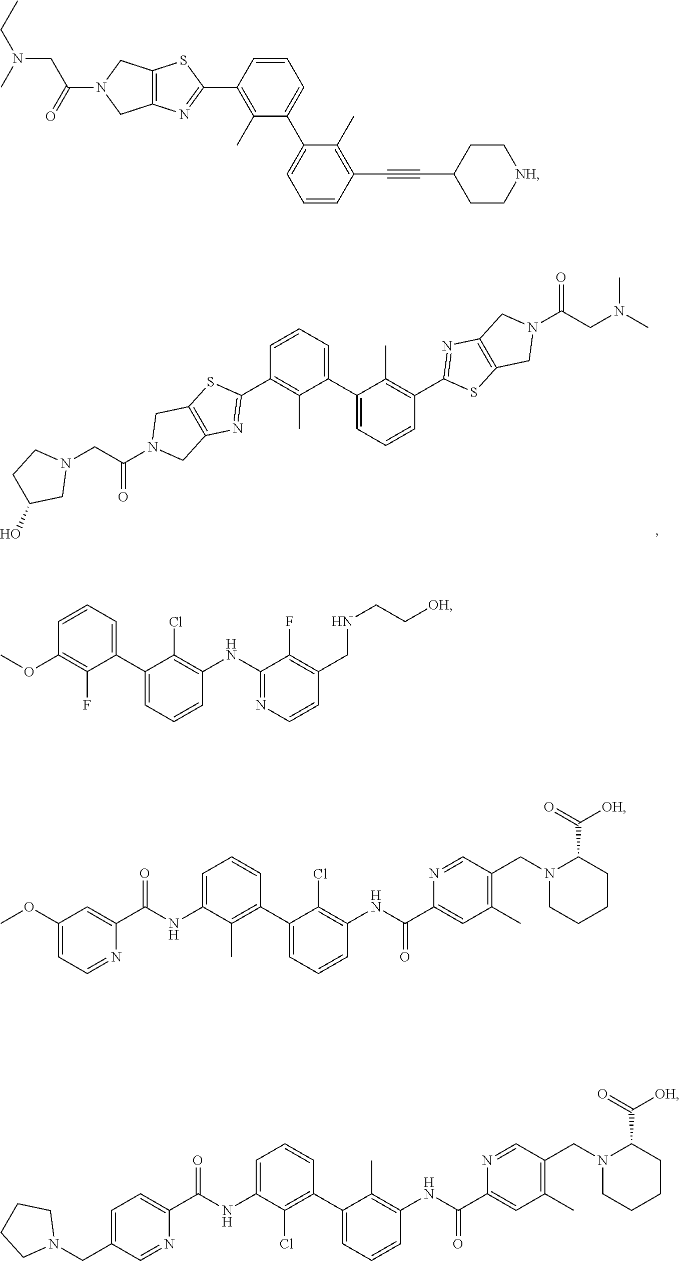 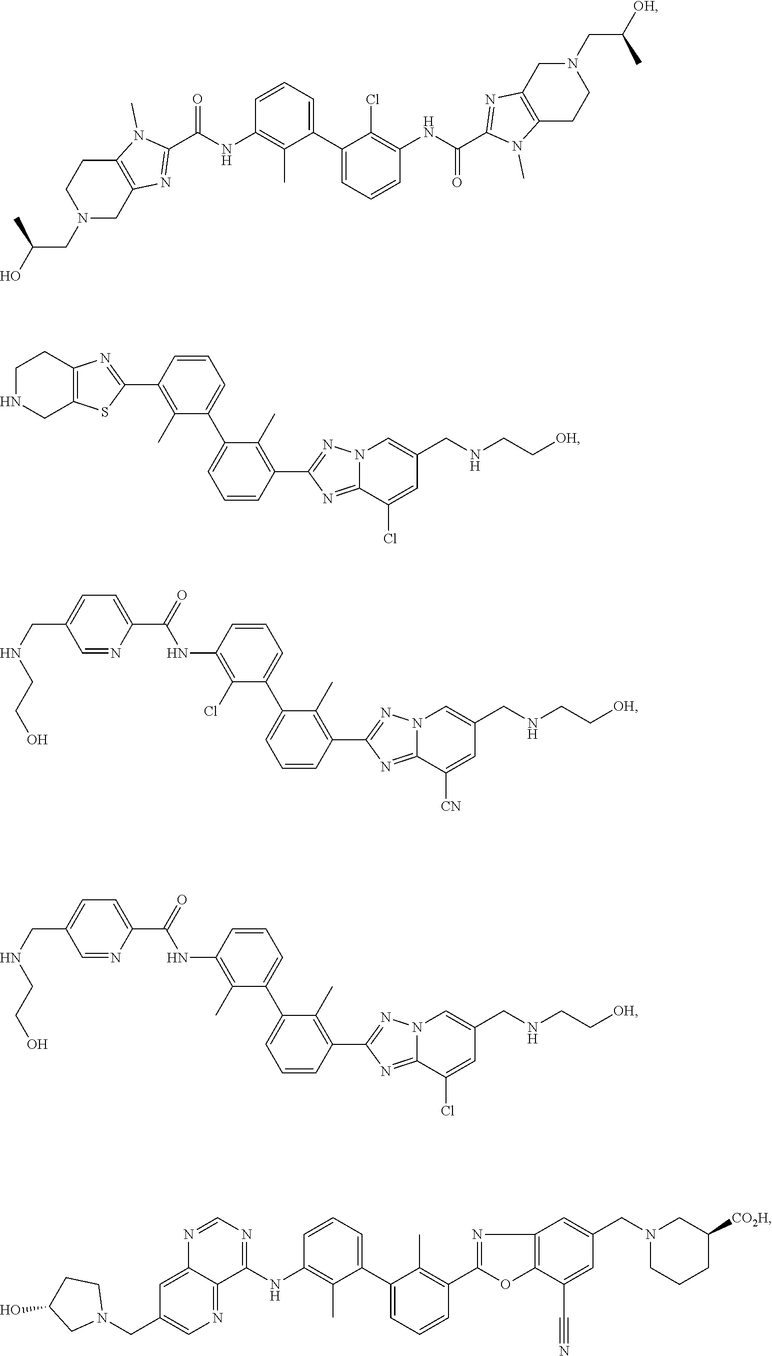 [SMILES: Cc1c(NC(=O)c2nc3c(n2C)CCN(C[C@H](C)O)C3)cccc1-c1cccc(NC(=O)c2nc3c(n2C)CCN(C[C@H](C)O)C3)c1Cl ; Cc1c(-c2nc3c(Cl)cc(CNCCO)cn3n2)cccc1-c1cccc(-c2nc3c(s2)CNCC3)c1C ; Cc1c(-c2nc3c(C#N)cc(CNCCO)cn3n2)cccc1-c1cccc(NC(=O)c2ccc(CNCCO)cn2)c1Cl ; Cc1c(NC(=O)c2ccc(CNCCO)cn2)cccc1-c1cccc(-c2nc3c(Cl)cc(CNCCO)cn3n2)c1C ; Cc1c(Nc2ncnc3cc(CN4CC[C@@H](O)C4)cnc23)cccc1-c1cccc(-c2nc3cc(CN4CCC[C@H](C(=O)O)C4)cc(C#N)c3o2)c1C] 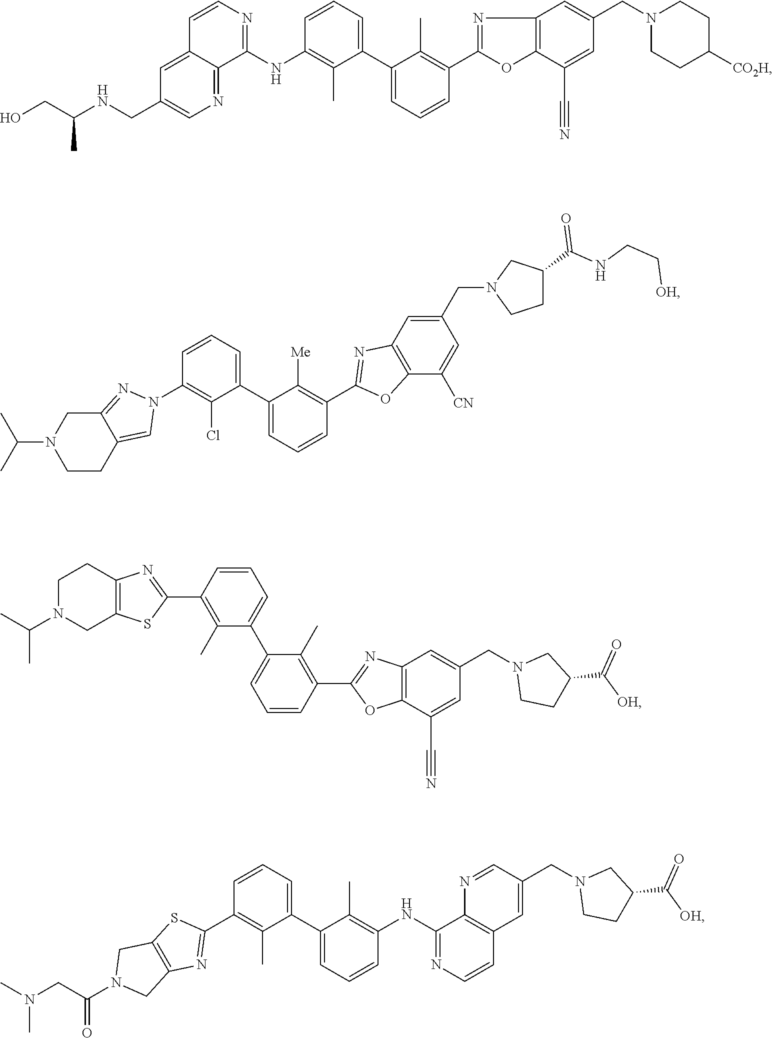 [SMILES: Cc1c(Nc2nccc3cc(CN[C@@H](C)CO)cnc23)cccc1-c1cccc(-c2nc3cc(CN4CCC(C(=O)O)CC4)cc(C#N)c3o2)c1C ; Cc1c(-c2nc3cc(CN4CC[C@@H](C(=O)NCCO)C4)cc(C#N)c3o2)cccc1-c1cccc(-n2cc3c(n2)CN(C(C)C)CC3)c1Cl ; Cc1c(-c2nc3cc(CN4CC[C@@H](C(=O)O)C4)cc(C#N)c3o2)cccc1-c1cccc(-c2nc3c(s2)CN(C(C)C)CC3)c1C ; Cc1c(Nc2nccc3cc(CN4CC[C@@H](C(=O)O)C4)cnc23)cccc1-c1cccc(-c2nc3c(s2)CN(C(=O)CN(C)C)C3)c1C] 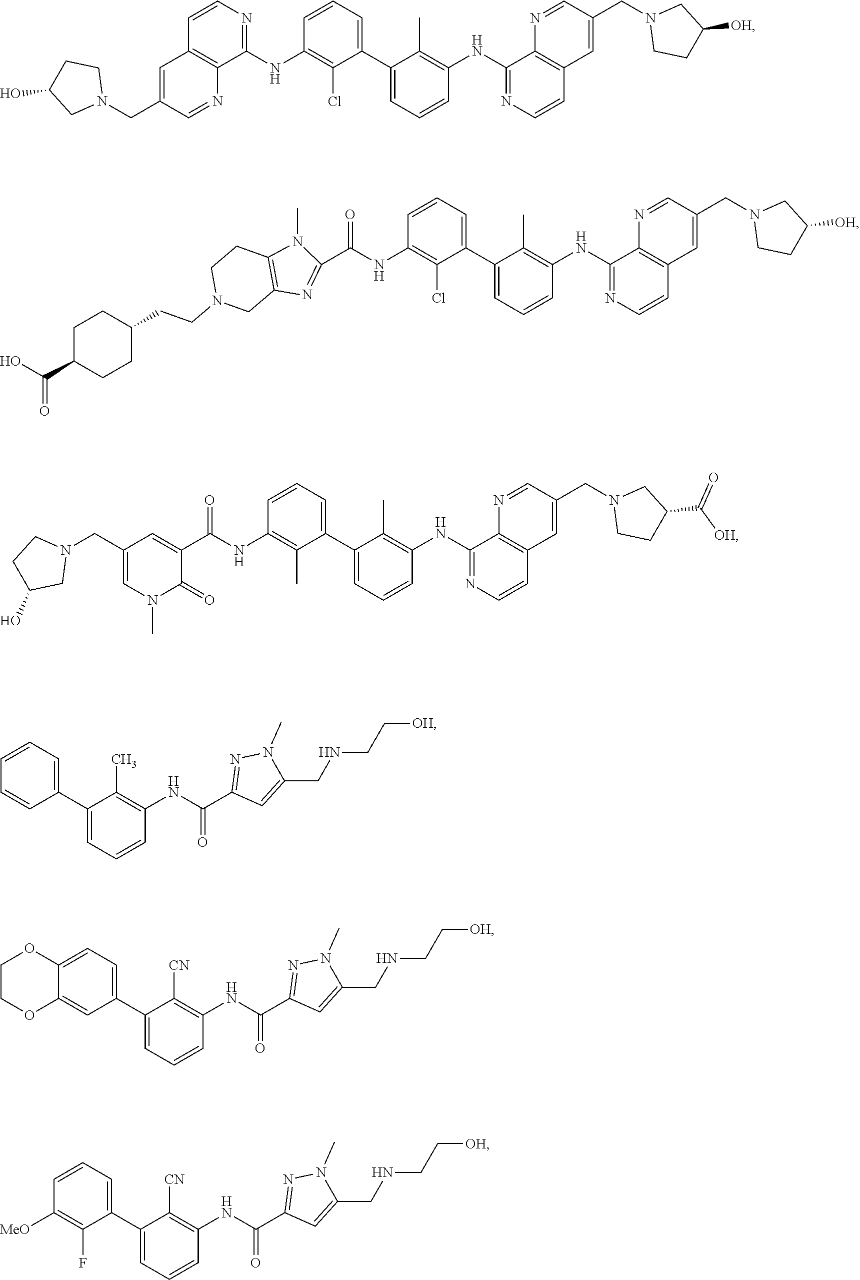 [SMILES: Cc1c(Nc2nccc3cc(CN4CC[C@H](O)C4)cnc23)cccc1-c1cccc(Nc2nccc3cc(CN4CC[C@@H](O)C4)cnc23)c1Cl ; Cc1c(Nc2nccc3cc(CN4CC[C@@H](O)C4)cnc23)cccc1-c1cccc(NC(=O)c2nc3c(n2C)CCN(CC[C@H]2CC[C@H](C(=O)O)CC2)C3)c1Cl ; Cc1c(NC(=O)c2cc(CN3CC[C@@H](O)C3)cn(C)c2=O)cccc1-c1cccc(Nc2nccc3cc(CN4CC[C@@H](C(=O)O)C4)cnc23)c1C ; Cc1c(NC(=O)c2cc(CNCCO)n(C)n2)cccc1-c1ccccc1 ; Cn1nc(C(=O)Nc2cccc(-c3ccc4c(c3)OCCO4)c2C#N)cc1CNCCO ; COc1cccc(-c2cccc(NC(=O)c3cc(CNCCO)n(C)n3)c2C#N)c1F] 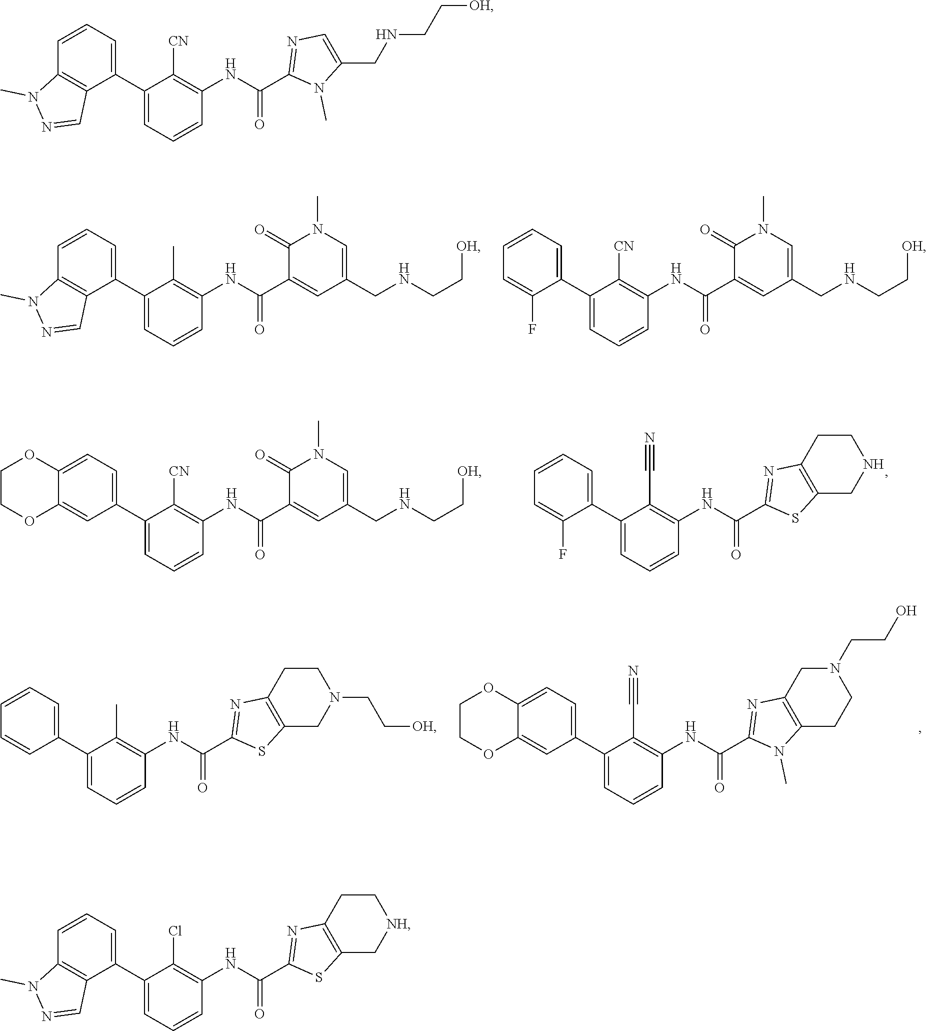 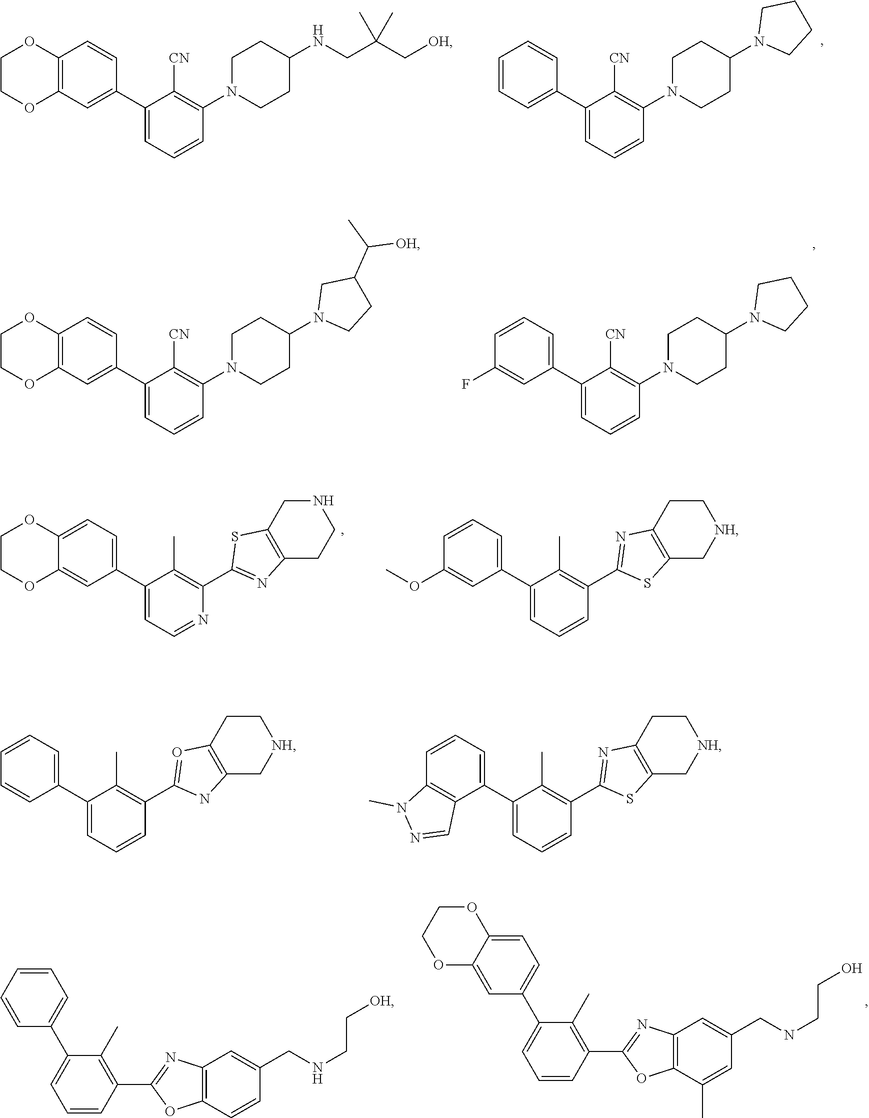 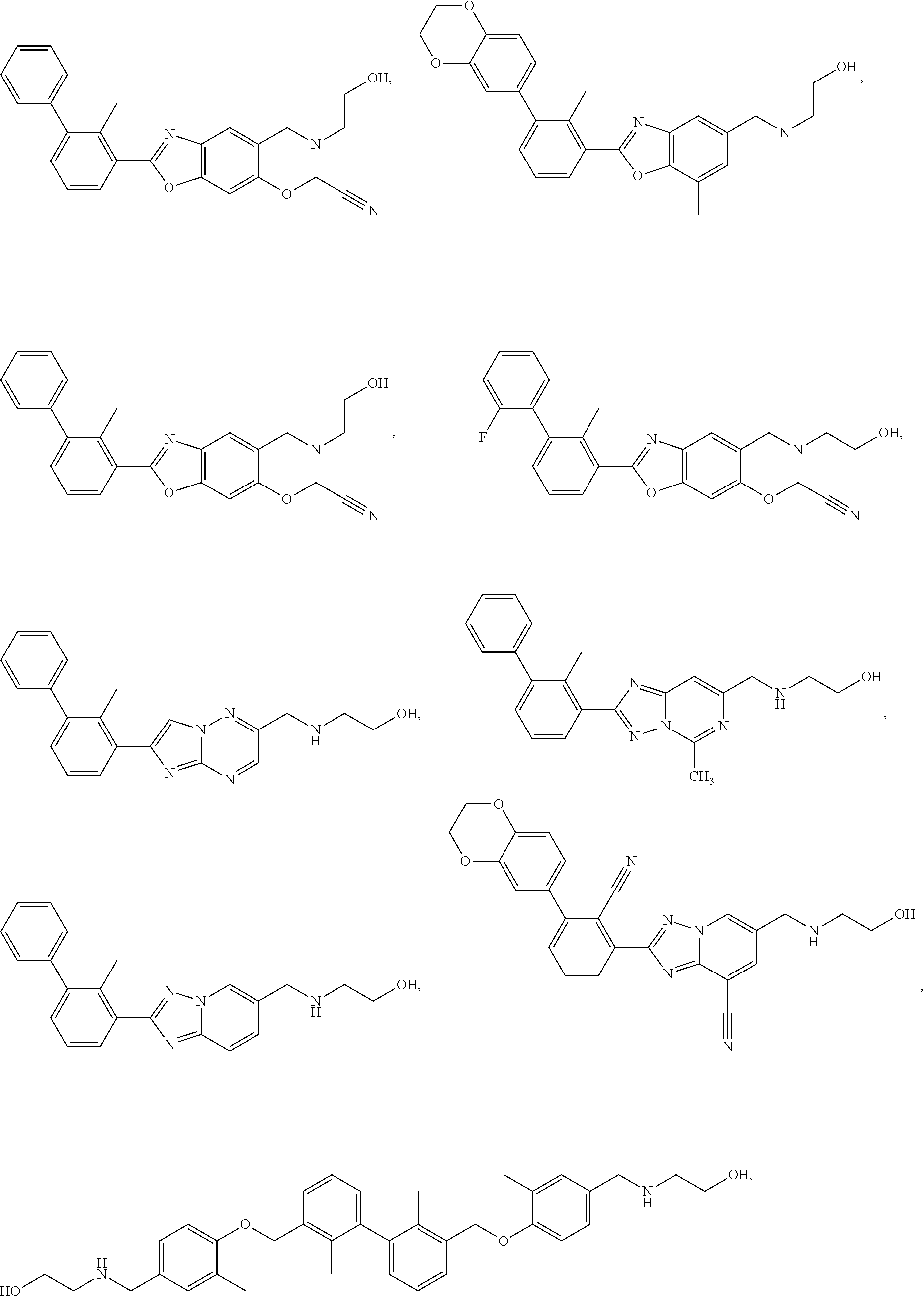 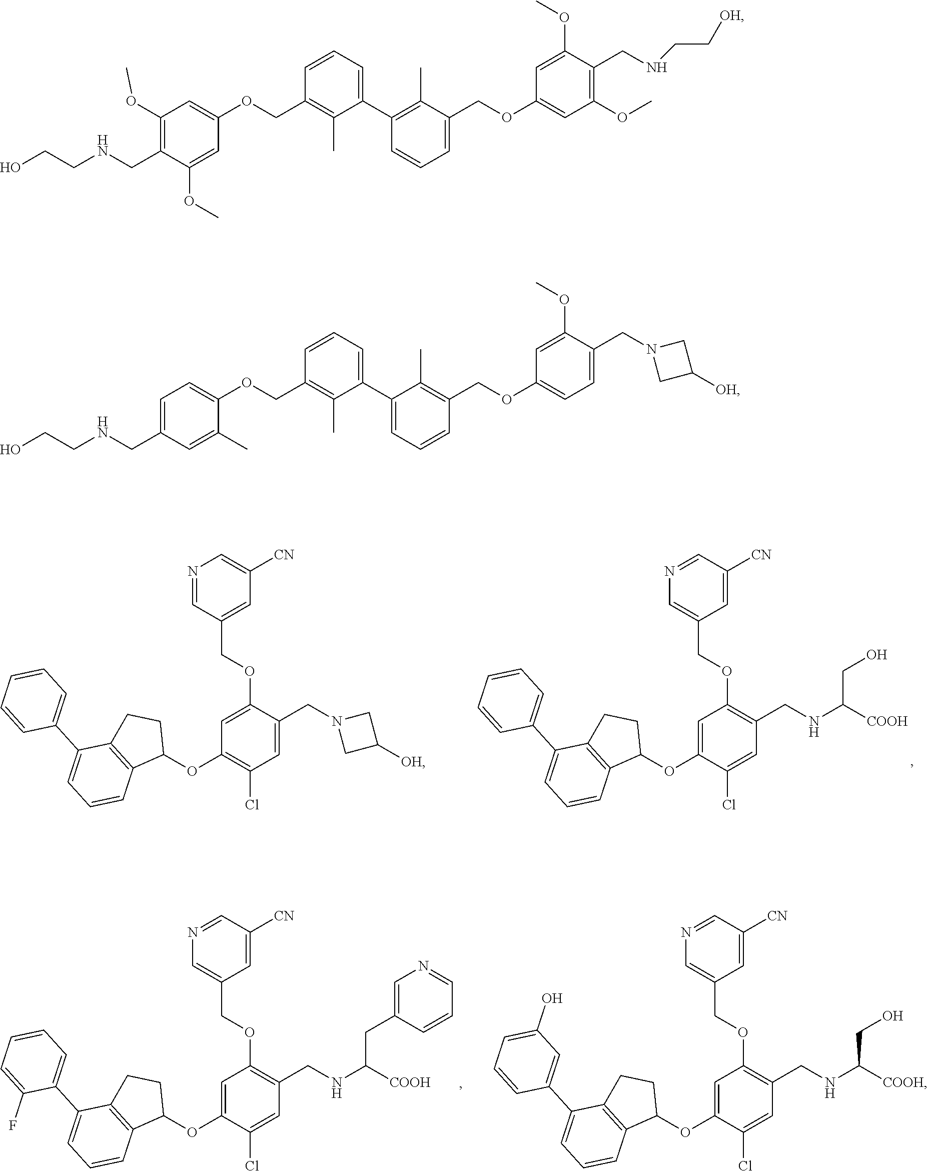 [SMILES: COc1cc(OCc2cccc(-c3cccc(COc4cc(OC)c(CNCCO)c(OC)c4)c3C)c2C)cc(OC)c1CNCCO ; COc1cc(OCc2cccc(-c3cccc(COc4ccc(CNCCO)cc4C)c3C)c2C)ccc1CN1CC(O)C1 ; N#Cc1cncc(COc2cc(OC3CCc4c(-c5ccccc5)cccc43)c(Cl)cc2CN2CC(O)C2)c1 ; N#Cc1cncc(COc2cc(OC3CCc4c(-c5ccccc5)cccc43)c(Cl)cc2CNC(CO)C(=O)O)c1 ; N#Cc1cncc(COc2cc(OC3CCc4c(-c5ccccc5F)cccc43)c(Cl)cc2CNC(Cc2cccnc2)C(=O)O)c1 ; N#Cc1cncc(COc2cc(OC3CCc4c(-c5cccc(O)c5)cccc43)c(Cl)cc2CN[C@@H](CO)C(=O)O)c1] 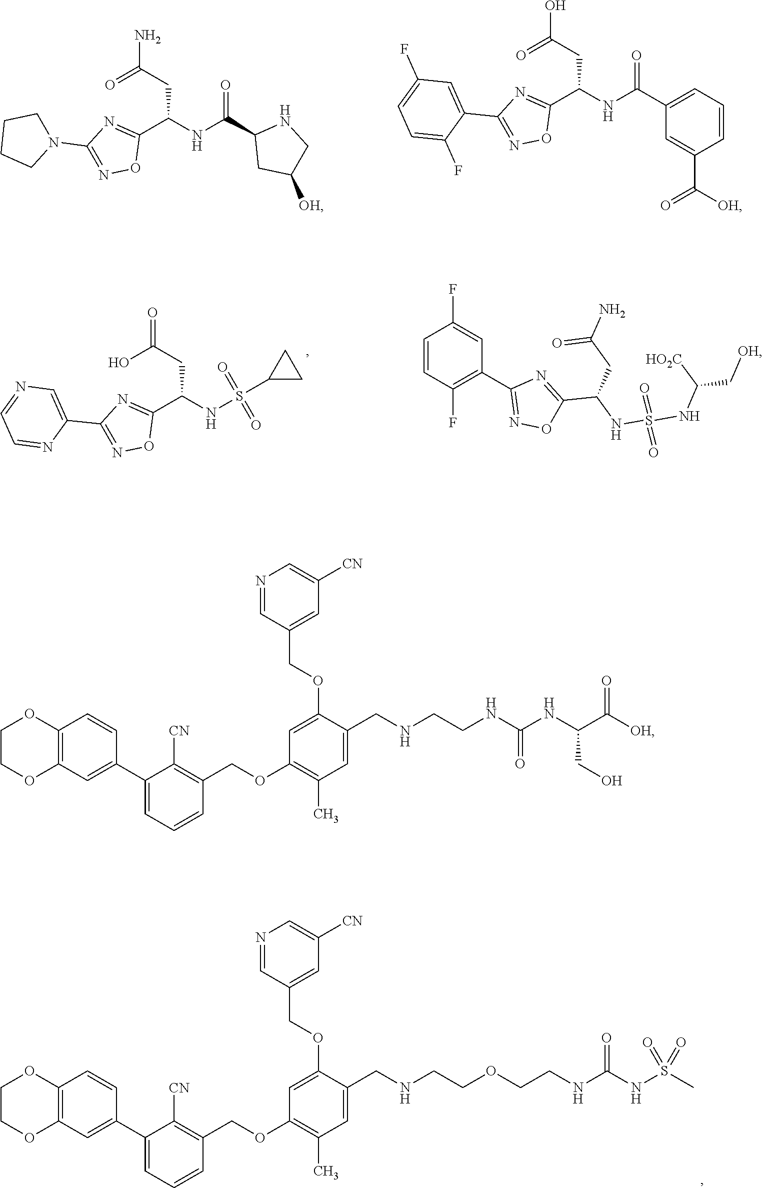 [SMILES: NC(=O)C[C@H](NC(=O)[C@@H]1C[C@H](O)CN1)c1nc(N2CCCC2)no1 ; O=C(O)C[C@H](NC(=O)c1cccc(C(=O)O)c1)c1nc(-c2cc(F)ccc2F)no1 ; O=C(O)C[C@H](NS(=O)(=O)C1CC1)c1nc(-c2cnccn2)no1 ; NC(=O)C[C@H](NS(=O)(=O)N[C@@H](CO)C(=O)O)c1nc(-c2cc(F)ccc2F)no1 ; Cc1cc(CNCCNC(=O)N[C@@H](CO)C(=O)O)c(OCc2cncc(C#N)c2)cc1OCc1cccc(-c2ccc3c(c2)OCCO3)c1C#N ; Cc1cc(CNCCOCCNC(=O)NS(C)(=O)=O)c(OCc2cncc(C#N)c2)cc1OCc1cccc(-c2ccc3c(c2)OCCO3)c1C#N] 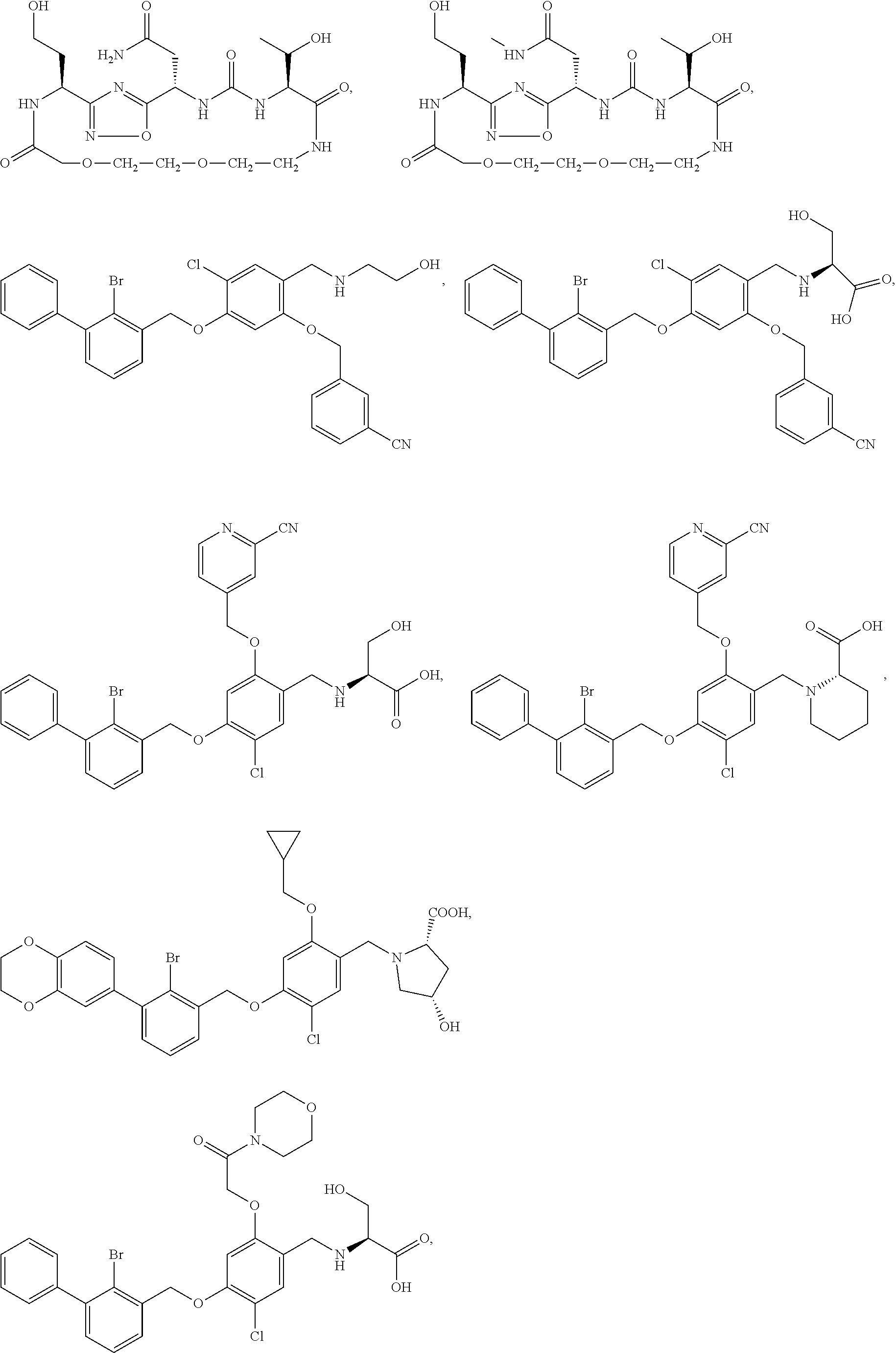 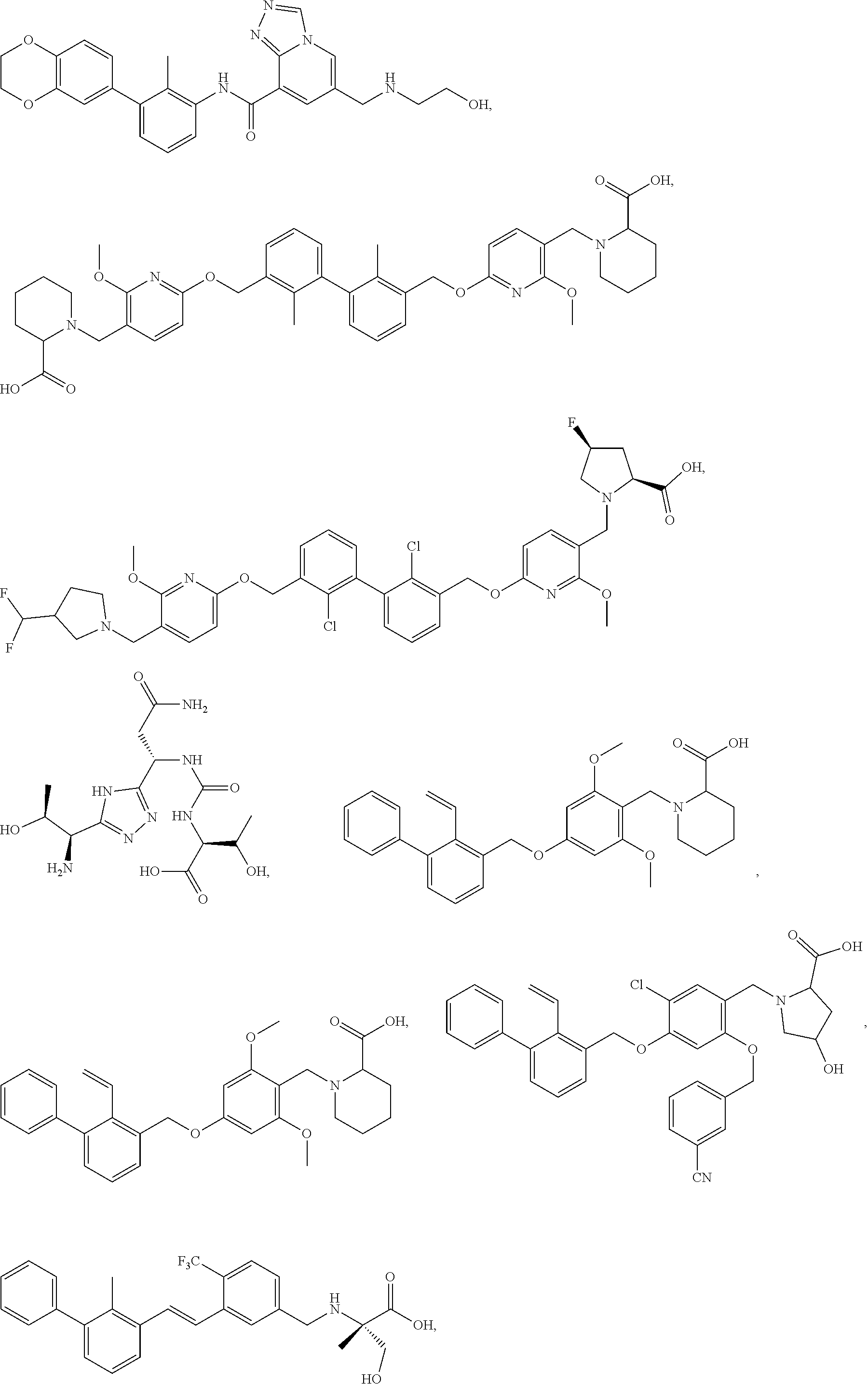 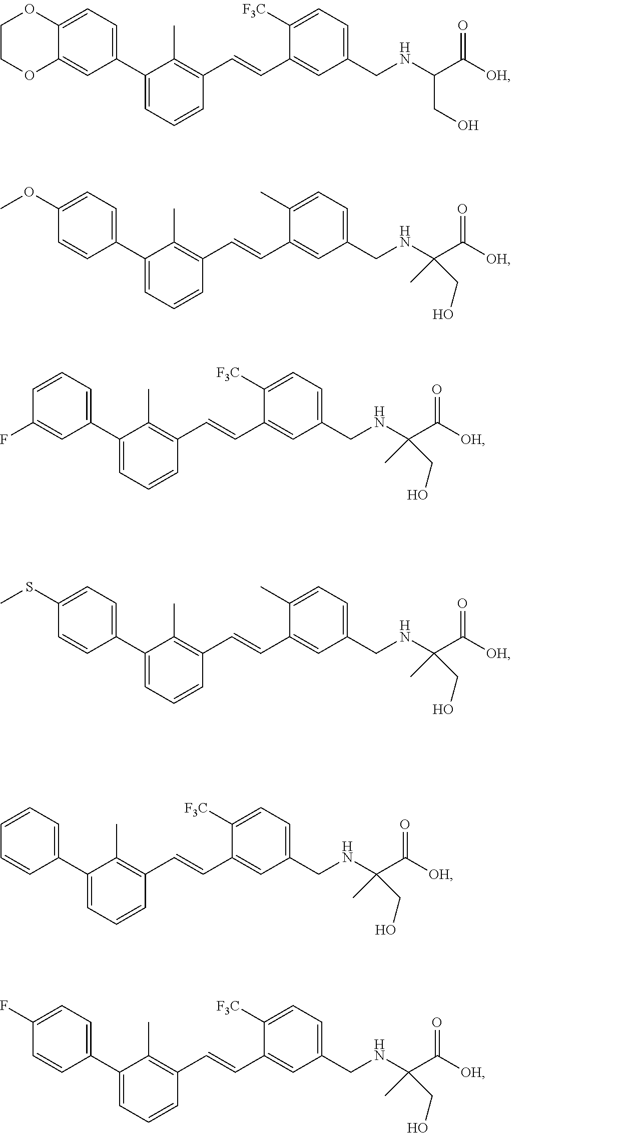 [SMILES: Cc1c(/C=C/c2cc(CNC(CO)C(=O)O)ccc2C(F)(F)F)cccc1-c1ccc2c(c1)OCCO2 ; COc1ccc(-c2cccc(/C=C/c3cc(CNC(C)(CO)C(=O)O)ccc3C)c2C)cc1 ; Cc1c(/C=C/c2cc(CNC(C)(CO)C(=O)O)ccc2C(F)(F)F)cccc1-c1cccc(F)c1 ; CSc1ccc(-c2cccc(/C=C/c3cc(CNC(C)(CO)C(=O)O)ccc3C)c2C)cc1 ; Cc1c(/C=C/c2cc(CNC(C)(CO)C(=O)O)ccc2C(F)(F)F)cccc1-c1ccccc1 ; Cc1c(/C=C/c2cc(CNC(C)(CO)C(=O)O)ccc2C(F)(F)F)cccc1-c1ccc(F)cc1] 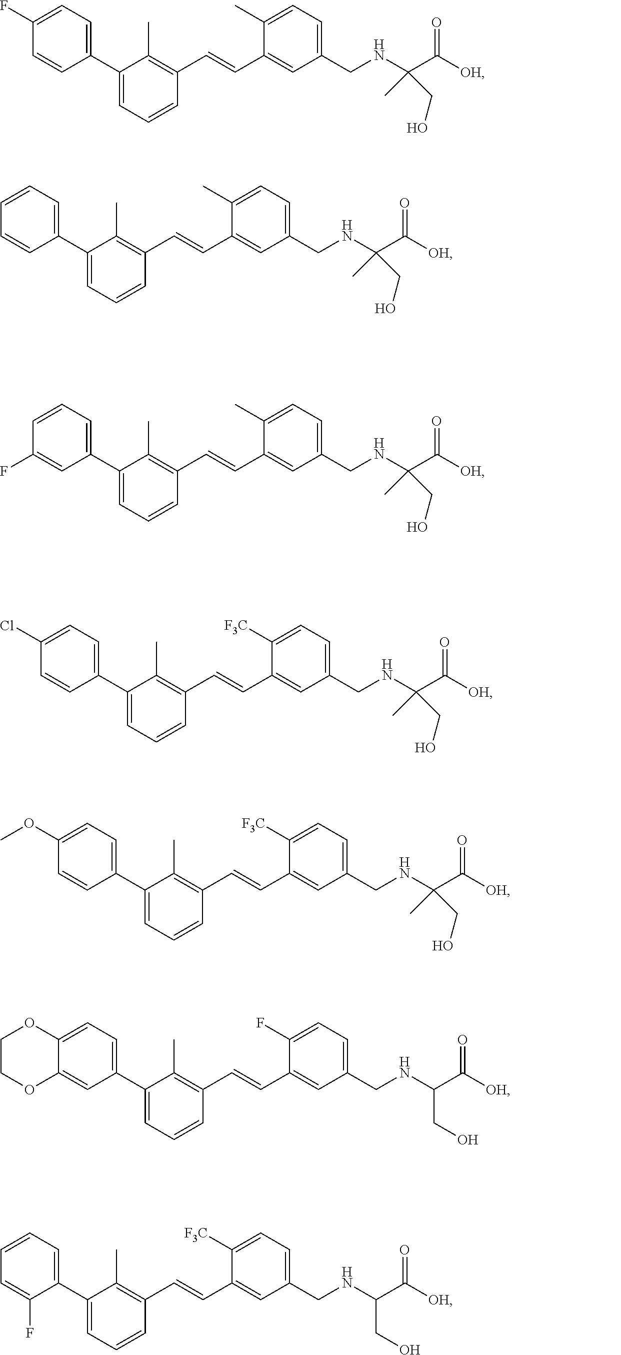 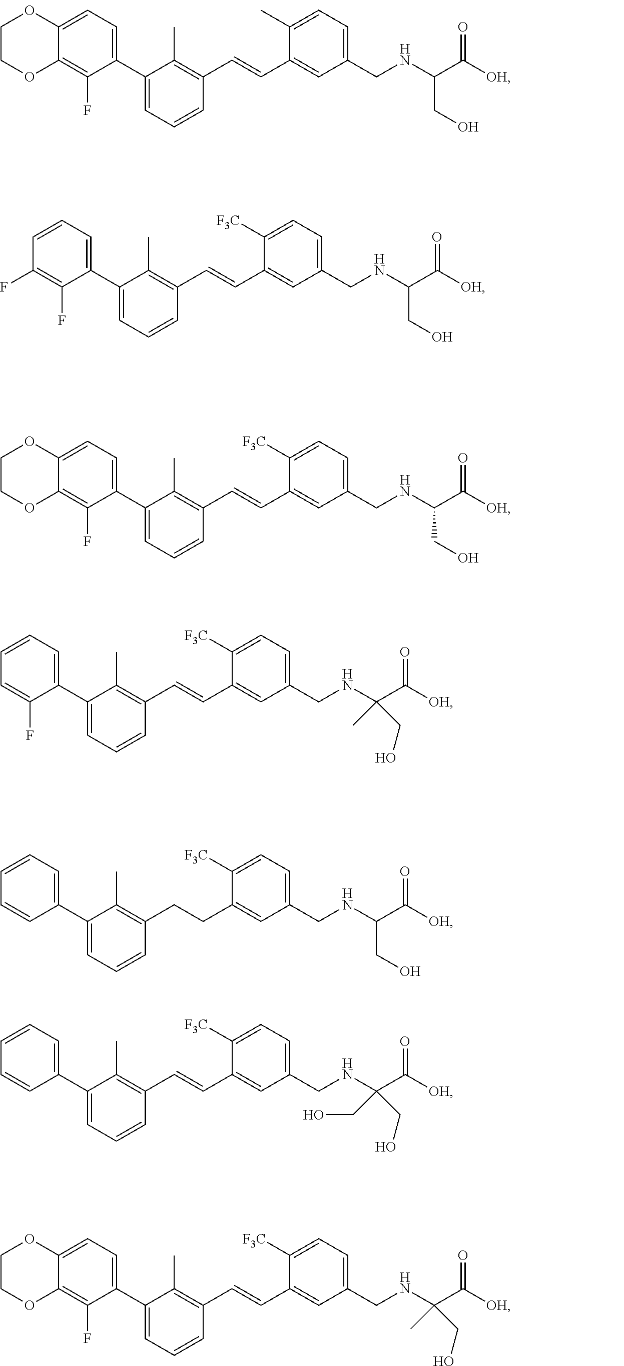 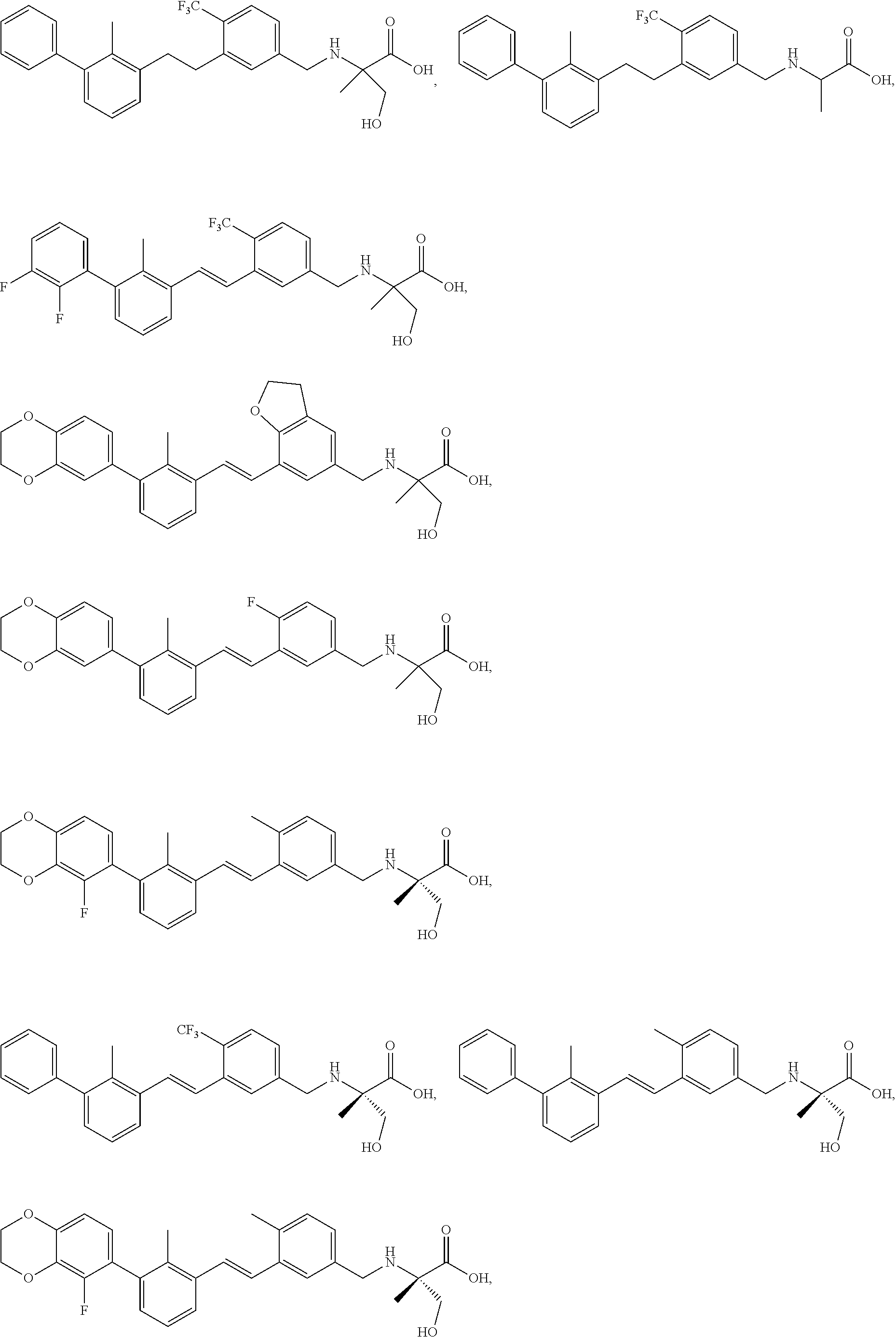 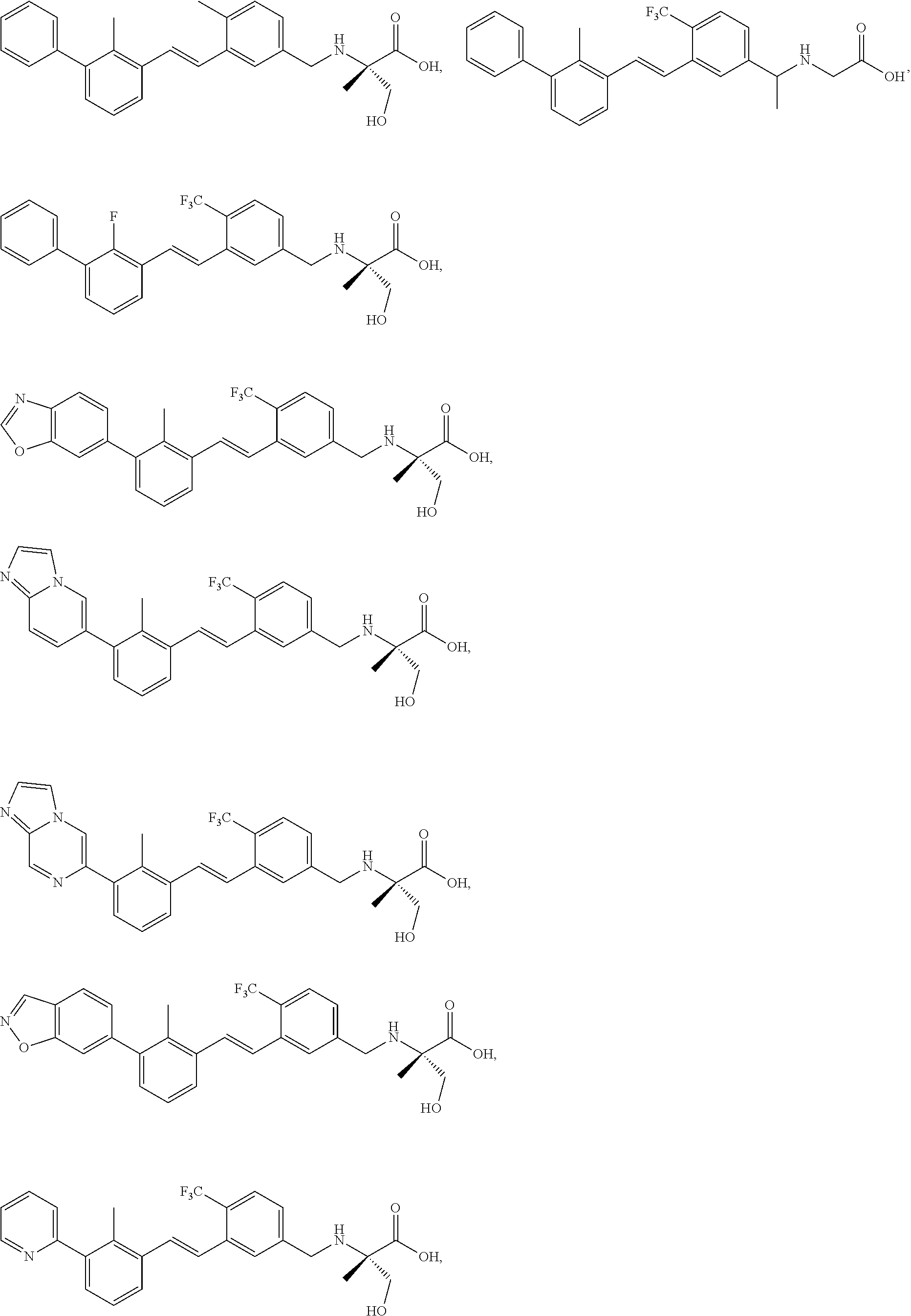 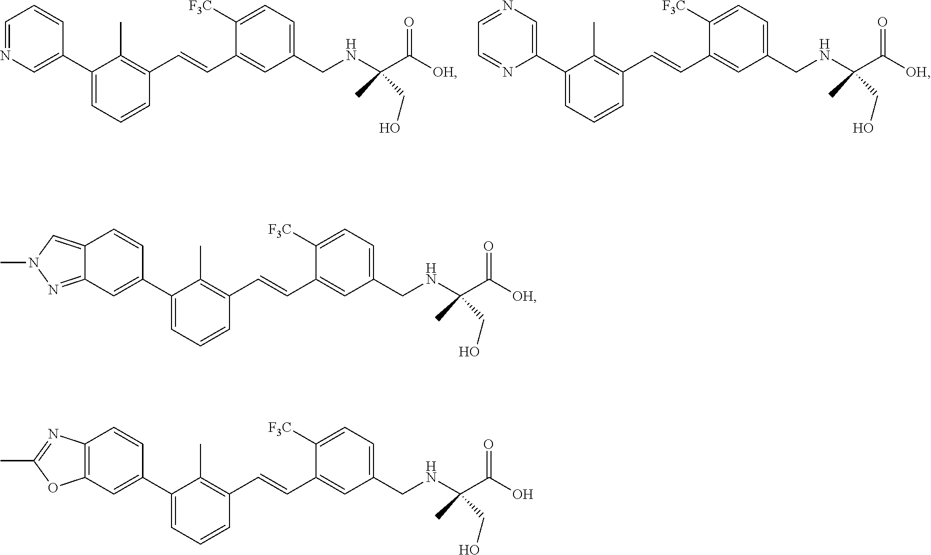
in free or pharmaceutically acceptable salt form.

7. The method of claim 1, wherein the compound is 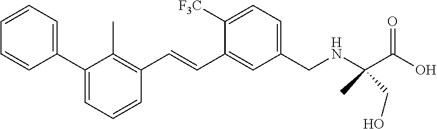 [SMILES: Cc1c(/C=C/c2cc(CN[C@@](C)(CO)C(=O)O)ccc2C(F)(F)F)cccc1-c1ccccc1]
in free or pharmaceutically acceptable salt form.

8. The method of claim 1, wherein the cancer is cervical carcinomas, renal cell carcinoma, melanomas, breast cancer, colorectal cancer, or head and neck squamous cell carcinoma (HNSCC).

9. The method of claim 1, wherein the cancer is breast cancer, melanomas or colorectal cancer.

10. (canceled)

11. The method of claim 1, wherein the compound is administered orally.

12. The method of claim 1, wherein the compound is administered at a total dose of 20-300 mg/kg or 30-240 mg/kg per day.

13. The method of claim 1, wherein the compound is administered at an amount of about 10-150 mg/kg or 15-120 mg/kg body weight twice a day (BID).

14. The method of claim 1, wherein the compound is administered at an amount of about 30 mg/kg, about 60 mg/kg or about 120 mg/kg body weight twice a day (BID).

15. The method of claim 1, wherein the subject has previously received cancer treatment;
and/or, the subject is a human;
and/or, the subject does not have a history of significant autoimmune disease;
and/or, the subject has not received organ or bone marrow transplants;
and/or, the subject has IFP of at least 20 mmHg, at least 30 mmHg, at least 40 mmHg, or at least 50 mmHg.

16. The method of claim 15, wherein the cancer treatment is chemotherapy, optionally wherein the chemotherapy comprises a platinum containing chemotherapeutic agent, optionally wherein the chemotherapy is platinum-containing doublet chemotherapy.

17. The method of claim 15, wherein the cancer treatment comprises administering an anti-PD-1 antibody to the subject, optionally wherein the anti-PD-1 antibody is pembrolizumab, nivolumab or cemiplimab.

18. The method of claim 15, wherein the cancer treatment comprises administering an anti-PD-L1 antibody to the subject, optionally wherein the anti-PD-L1 antibody is atezolizumab, durvalumab, or avelumab.

19. The method of claim 15, wherein the subject is not responsive to the cancer treatment.

20-22. (canceled)

23. The method of claim 1, wherein the IFP is measured by a micropuncture technique, a wick-in-needle technique or MRI technology.

24. The method of claim 1 further comprising administration of an additional active agent selected from at least one of an antibody binding to PD-1 or PD-L1, an inhibitor of the CTLA-4/B7 interaction, or an inhibitor binding to vascular endothelial growth factor (VEGF).

25-27. (canceled)
